(19) United States
(12) Patent Application Publication
(10) Pub. No.: US 2022/0202739 A1
(43) Pub. Date: Jun. 30, 2022

(54) ENDOCANNABINOID MIMETIC AND ANTI-INFLAMMATORY COMPOUND CONTAINING COMPOSITIONS, METHODS OF PREPARATION AND USES THEREOF

(71) Applicant: PHARMA COSMETIX RESEARCH, LLC, North Chesterfield, VA (US)

(72) Inventors: Joseph A. Lewis, II, Chesterfield, VA (US); David H. McDaniel, Virginia Beach, VA (US); Lavinia Popescu, Jackson Heights, NY (US)

(21) Appl. No.: 17/697,046

(22) Filed: Mar. 17, 2022

Related U.S. Application Data
(63) Continuation of application No. PCT/US2020/051544, filed on Sep. 18, 2020.
(60) Provisional application No. 62/902,291, filed on Sep. 18, 2019.

(51) Int. Cl. A61K 31/085; A61K 31/12; A61K 36/61; A61K 36/185; A61K 36/54; A61K 36/53; A61K 31/365; A61K 31/201; A61K 31/192; A61K 31/122; A61K 31/05; A61K 31/16; A61P 17/02

(52) U.S. Cl. CPC A61K 31/085; A61K 31/12; A61K 36/61; A61K 36/185; A61K 36/54; A61K 36/53; A61P 17/02; A61K 31/201; A61K 31/192; A61K 31/122; A61K 31/05; A61K 31/16; A61K 31/365

(57) ABSTRACT

A composition comprising direct, indirect and related pathway endocannabinoid mimetic compounds and methods of using the composition.

### CROSS REFERENCE TO RELATED APPLICATIONS

This application is a continuation of International Application No. PCT/US2020/051544, filed on Sep. 18, 2020, and claims benefit to U.S. provisional Application 62/902,291, filed on Sep. 18, 2019, which applications are hereby incorporated by reference herein. The International Application was published in English on Mar. 25, 2021 as WO 2021/055785 A1 under PCT Article 21(2).

### FIELD OF THE INVENTION

The present invention relates to cosmetic compositions and pharmaceutical drug compositions intended for either human or animal use.

### BACKGROUND OF THE INVENTION

The endocannabinoid system (ECS) is a complex biological evolutionarily conserved homeostatic signaling network composed of receptors, ligands, enzymes and transport proteins. Endocannabinoid ligands include endogenously produced lipids that activate two distinct “direct” endocannabinoid receptors, CB1 and CB2. Known examples of such lipid ligands include N-arachidonoylethanolamine [anandamide (AEA)] and 2-arachidonoylglycerol (2-AG). However, other lipids have an indirect effect on the ECS system, e.g. N-palymitoylethanolamide and N-oleoylethanolamine and there are other cannabinoid ligands, the most used and studied being those derived from C. sativa, e.g. THC and CBD; and less familiar small molecule G-protein coupled endocannabinoid phytomimetic activators, e.g. trans-beta caryophyllene (B-caryophyllene), curcumin, and honokiol. However, ECS receptors also include nonclassical types of receptors, such as the ionotropic TRP pain receptors, inflammatory nuclear PPAR receptors, inflammatory cytokine receptors e.g. IL, TNFa and NFKB, inflammatory enzymatic receptors e.g. COX, LOX, iNOS and MMP, and other ECS related ispathway targets, e.g. GlyR, CERS, CASP8, MAPK/ERK. Numerous enzymes, e.g. DAGL, NAPE-PLD, MAGL and FAAH, modulate the metabolism of AEA and 2-AG ligand synthesis and degradation. In addition, a number of transport proteins (eCBTs) assist water and lipid soluble passage from intercellular cytoplasm through cell membrane to extracellular matrix. (See FIG. 1.)

Cannabinoids, i.e., compounds derived from Cannabis sativa, interact with the endocannabinoid system. There are more than 60 known cannabinoids produced by C. sativa. Although such compounds can be useful for influencing the endocannabinoid system, some cannabinoids can elicit undesirable side effects such as dizziness, euphoria, and addiction. In addition, C. sativa and its derived compounds, including cannabidiol (CBD), are classified as drugs and/or controlled substances in many countries including the USA and therefore can be strictly regulated.

### BRIEF SUMMARY OF THE INVENTION

Applicants have now discovered that compositions comprising a diverse blend of direct and indirect endocannabinoid mimetic compounds and ECS related pathway anti-inflammatory compounds, some or all of which may be derived from natural plant extracts, are effective in combating a broad array of undesirable ECS modulated biological processes, common to humans and other mammals, many of which are particularly targeted to skin conditions (e.g., those mentioned hereunder), and some of which may have applications in a wide variety of human body disorders, especially those related to dysfunction of any organ or tissue of the human body under influence by the ECS, and in some cases even as a result of genetic defects or disease. In some embodiments, the compositions are effective in providing one or more of inflammation, pain, and itch relief. In some embodiments, the compositions are effective at modulating one or more of wound healing, mitigating skin matrix dysfunction, modulating cellular proliferation, differentiation, autophagy, apoptosis, and senescence, lipid deposition and barrier function, and skin microbiome. Effects of the compositions can be agonistic or antagonistic. Certain combinations can modulate gene expression to produce therapeutic benefits for cancer and stem cells. In aspects, the invention includes direct and indirect endocannabinoid mimetic and anti-inflammatory compounds and/or natural extracts containing such compounds, and blends thereof, covering topical or systemic routes of delivery, and their use to treat and/or prevent damage to any organ of a mammalian species, including human skin, caused by, e.g., any dysfunction or homeostasis imbalance of the human endocannabinoid system (ECS), oxidative and inflammatory stress and resulting degenerative processes, or both, and to also or alternatively provide pain relief, and to correct skin matrix and barrier dysfunction with improved wound healing effects and including positive mediation effects on skin microbiome. Such compositions may be formulated for or applied in topical cosmetic use.

Other compositions may be intended solely for topical therapeutic drug use. Such compositions may have use as, e.g., systemic drugs. Some compositions provide anti-inflammatory effects, broad protection from undesirable oxidative processes that affect skin, wound healing and pain relief in the convenience of a single composition. Other compositions also or alternatively provide anti-inflammatory effects, broad protection from undesirable oxidative processes that affect skin, restoration of skin ECS homeostasis and improvement in skin matrix, cell senescence, skin barrier, and skin microbiome. Compositions can have applications for, e.g., one or more of skin prejuvenation, rejuvenation, and regeneration. Other compositions provide therapeutic effects on human body disorders where the human ECS may play a critical role, to mitigate negative effects from either genetic defects or disease state.

In one embodiment, the invention provides a composition comprising:
a) at least one direct endocannabinoid mimetic compound, wherein each compound detectably or significantly modulates gene expression of a CB1 and/or CB2 gene;
b) at least one indirect endocannabinoid mimetic compound, wherein each compound:
   1) detectably or significantly modulates gene expression of FAAH; and/or
   2) detectably or significantly modulates gene expression of MAGL;
c) at least one ECS related pathway anti-inflammatory compound, wherein each compound
   1) detectably or significantly modulates gene expression of PPARgamma (PPARg), PPARalpha (PPARa), PPARbeta (PPARb), or any combination thereof; and/or
   2) detectably or significantly modulates gene expression of COX1 (i.e., PTGS1), COX2, iNOS, 5-LOX, 12-LOX, a matrix metalloprotease (MMP), or any combination thereof; and/or
   3) detectably or significantly modulates gene expression of IL-1beta, IL-1alpha (IL1a), IL-6, IL-8, NFKappaBeta (NFKB), TNFalpha (TNFa), IL-10, or any combination thereof; and
d) at least one ECS related TRP pathway compound, wherein each compound detectably or significantly modulates gene expression of TRPA1, TRPM8, TRPV1, TRPV3, TRPV4, TRPV6, or any combination thereof,
and wherein gene expression in each case is measured in a cell exposed to the compound and is compared to the gene expression in a cell not exposed to the same compound.

In another embodiment, the invention provides a composition comprising:
a) at least one direct endocannabinoid mimetic compound, wherein each compound detectably or significantly increases expression of a CB1 and/or CB2 gene; and
b) at least one indirect endocannabinoid mimetic compound, wherein each compound
   1) detectably or significantly decreases gene expression of FAAH; and/or
   2) detectably or significantly decreases gene expression of MAGL; and
c) at least one ECS related pathway anti-inflammatory compound, wherein each compound
   1) detectably or significantly increases gene expression of PPARg, PPARa, PPARb, or any combination thereof; and/or
   2) detectably or significantly decreases gene expression of COX1 (i.e., PTGS1), COX2, iNOS, 5-LOX, 12-LOX, a matrix metalloprotease (MMP), or any combination thereof; and/or
   3) detectably or significantly decreases gene expression of IL-1beta, IL-1alpha(IL1a), IL-6, IL-8, NFKappaBeta (NFKB) TNFalpha (TNFa), increases expression of IL-10, or any combination thereof; and
d) at least one ECS related TRP pathway compound, wherein each compound detectably or significantly decreases gene expression for TRPV1, decreases gene expression for TRPV3, detectably or significantly increases gene expression for TRPV4, increases gene expression for TRPV6, detectably or significantly increases gene expression for TRPA1, detectably or significantly increases gene expression for TRPM8, or any combination thereof; and
wherein gene expression in each case is measured in a cell exposed to the compound and is compared to the gene expression in a cell not exposed to the compound.

### BRIEF DESCRIPTION OF THE DRAWINGS

FIG. 1: Non-limiting overview of the complex endocannabinoid system of ligands, receptors, enzymes and transporter proteins.

FIG. 2: Simplified ECS pathway flowchart showing direct and indirect endocannabinoid pathways, and ECS related pathways including enzymatic, nuclear and cytokine inflammatory pathways, the arachidonic acid inflammatory cascade, ECS related TRP pathways and GlyR pain dependent pathways and pathways related to skin matrix function including CERS, CASP8, MAPK/ERK.

### DETAILED DESCRIPTION OF THE INVENTION

### Definitions

Endocannabinoid Mimetic Compound: Any isolated compound that acts as a direct ligand for CB1 or CB2 receptor, indirectly modulates the ECS via metabolism of AEA and/or 2AG or modulates an ECS related pathway, wherein the compound is not sourced from Cannabis sativa or any Cannabis species, subspecies or hybrid; e.g. beta caryophyllene sourced from Bidens pilosa, Syzygium aromaticum, Eugenia caryophyllata, Piper nigrum, Perilla frutescens, Rosmarinus officinalis, Lindera benzoin, Centella asiatica, Angelica archangelica, Coleus barbatus, Origanum vulgare, Copaifera officinalis, and Pogostemon cablin (not C. sativa) qualifies under this definition.

Cannabinoid Compound: Any of the more than 60 compounds found in any part of a cannabis plant, including Cannabis sativa, Cannabis sativa indica, as well as hybrids, intergenetic hybrids, genetically modified plants, and plants derived from stems cells isolated from any part of a suitable cannabis plant including cannabigerol, delta-9-tetrahydrocannabinol, cannabidiol, cannabichromene, cannabinol, delta-8-tetrahydrocannabinol, cannabicyclol, cannabinodiol, cannabielsoin, and cannabitriolany of the more than 60 known compounds found in any part of a cannabis plant; e.g. cannabigerol, delta-9-tetrahydrocannabinol, cannabidiol, cannabichromene, cannabinol, delta-8-tetrahydrocannabinol, cannabicyclol, cannabinodiol, cannabielsoin, and cannabitriol.

Endocannabinoid Phytomimetic Compound (or activator): Any endocannabinoid mimetic compound that is derived from plants and is not an endogenous direct endocannabinoid ligand (such as AEA and 2AG).

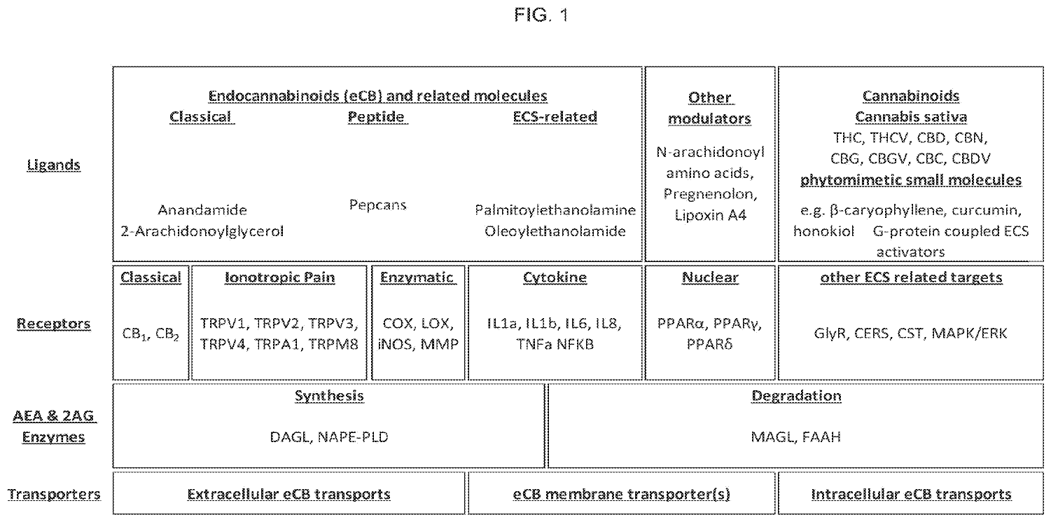

Direct Endocannabinoid Compound: endogenous ligands AEA or 2AG or any isolated compound with a direct agonistic or antagonistic binding function for CB1 or CB2 receptors. See, e.g., FIGS. 1 & 2.

Indirect Endocannabinoid Compound: isolated compounds that affect metabolism of AEA or 2AG. See, e.g., FIGS. 1 & 2.

ECS Ligand: Endogenous endocannabinoids, e.g., AEA and 2AG, Endocannabinoid Mimetic Compounds and Cannabinoid Compounds including fatty amides and small molecule G-protein coupled Endocannabinoid Phytomimetic Compounds. See, e.g., FIG. 1.

ECS Receptor: Any receptor that modulates the ECS; including, direct CB1 and CB2 receptors, endogenous ligand metabolism enzyme receptors, TRP ionotropic receptors, inflammatory cytokine receptors, inflammatory nuclear receptors, and non-traditional cannabinoid targets that modulate other ECS related pathways. See, e.g., FIG. 1.

ECS Enzyme: Any enzyme that modulates endocannabinoid metabolism of AEA or 2AG or any enzyme that modulates related ECS pathways. See, e.g., FIG. 1.

Transporter Protein: (eCBTs) proteins that assist water and lipid soluble passage from intercellular cytoplasm through cell membrane to extracellular matrix. See, e.g., FIG. 1.

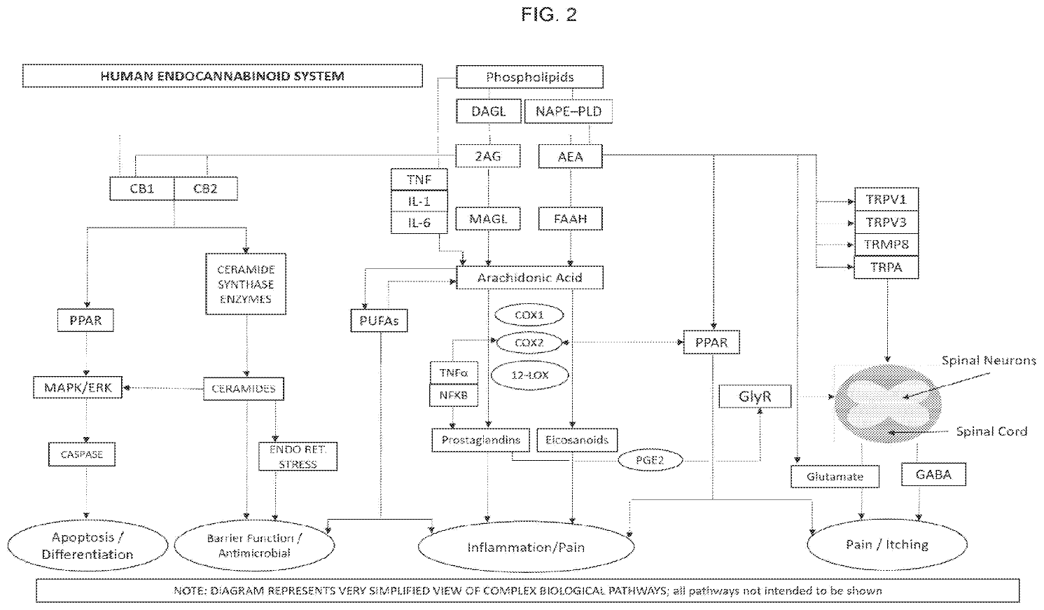

ECS Pathway: Any pathway related to the human endocannabinoid system, e.g. direct, indirect, or related pathway. See, e.g., FIG. 2.

ECS Related Ligands: Any ECS ligands that are not direct CB1 or CB2 receptor agonists or antagonists or do not modulate AEA or 2 AG metabolism. Typically, ligands for receptors that affect ECS Related Pathways.

ECS Related Pathway: Any ECS pathway involving receptors outside of the direct endocannabinoid CB1 & CB2 receptors and the indirect endocannabinoid metabolism of AEA or 2AG. Examples of such ECS pathways include cytokine, enzyme or nuclear anti-inflammatory pathways, TRP pain and cellular modulation pathways, ceramide synthase barrier function and antimicrobial pathways, and MAPK/ERK/caspase pathways affecting cellular proliferation, differentiation, autophagy, apoptosis, and senescence. See, e.g., FIG. 2.

ECS Related TRP Pathway: Any ECS pathway(s) that modulate expression of one or more TRP genes. In exemplary aspects, such a pathway is a TRP group 1 pathway, in which one or more of TRPC (“C” for canonical), TRPV (“V” for vanilloid), TRPM (“M” for melastatin), TRPN and TRPA are modulated.

ECS Cell or ECS Cell Type: any cell type detectably or significantly influenced by the human endocannabinoid system, e.g. keratinocytes, fibroblasts, melanocytes, sebocytes, adipocytes, langerhans cells, dermal papillae cells, dendritic cells, macrophages, mast cells, various T cell populations and also endothelial and vascular cells, and merkle cells.

Definitions for Routes of Administration:

1. Topical includes any suitable form of application to the skin surface or mucosa, including, but not limited to direct product application, by hand, microneedles, patch, or roller.
2. Systemic includes any suitable form of systemic administration, including, but not limited to, intramuscular, intravenous, subcutaneous, intraarterial, intradermal and intraperitoneal injection, inhalation, intranasal, sublingual, via implants or patches, and oral.

Cosmetic: articles for cleansing, beautifying, promoting attractiveness, or altering the appearance of an organism, particular a person, and which is not a drug.

Compounds: refers to chemical compounds. The terms “compound” and “class,” when used in reference to a compound, are intended to encompass their broadest reasonable scope. As an example, the chemical class of monoterpene includes monoterpenoids.

Natural Extract: any suitable composition obtained/derived from, or combination of such compositions, or part, of a fruit, spice, vegetable, root, leaf, flower, husk, stem, animal tissue, or other extracts (e.g., natural extracts) identified by source genus and species that contains an effective amount of a compound as used herein (an amount that causes a detectable or significant amount of one or more of the effects described herein). In certain embodiments the natural extract may be derived from a genetically modified, environmentally influenced, or climatically influenced species.

Compositions: combinations of one or more compounds, natural extracts, or any combination thereof, that detectably or significantly modulates one or more pathways of the ECS system.

Prejuvenation: prophylactic treatment to prevent the onset or reduce the frequency, severity, duration and/or magnitude of detrimental changes to an organism, cells, or an organ (e.g., the skin), typically associated with a condition (e.g., aging, sun exposure, inflammation, and other conditions described herein).

Rejuvenation: correction of detrimental changes to a healthy or detectably/significantly healthier state.

Regeneration: detectable or significant production of new cells/tissue to replace cells damaged or killed in association with detrimental changes.

Skin matrix: all elements comprising the cellular or subcellular components of the skin and subcutaneous tissues including but not limited to the stratum corneum, the epidermis, the dermal matrix (including the entire components of the dermis such as fibroblasts, keratinocytes, vasculature, neural structures, adnexal structures), and the subcutaneous tissues and component cells thereof, such as adipocytes).

Intrinsic Aging: qualitative and/or quantitative (and often significant) skin changes that result from declining physiologic functions and capabilities, such as diminished or defective synthesis of collagen and elastin in the dermis, increased dryness, flattened papillary dermis, decreased stratum corneum turnover.

Extrinsic Aging: qualitative and/or quantitative (and often significant) skin changes that result from external factors such as ultra-violet radiation (photoaging), cigarette smoking, and air pollution among others.

Homeostasis: the state of steady internal, physical, and chemical conditions maintained by healthy living systems. Typically a condition of optimal functioning for the organism. Homeostasis can be characterized by several variables or range(s) thereof (e.g., not significantly deviating therefrom), e.g., body temperature and fluid balance, being kept within certain pre-set limits (homeostatic range(s)). Other variables that characterize homeostasis can include the pH of extracellular fluid, the concentrations of sodium, potassium and calcium ions, as well as that of the blood sugar level, and these need to be regulated despite changes in the environment, diet, or level of activity.

Autophagy a natural, regulated mechanism of the cell that removes unnecessary or dysfunctional components, allowing orderly degradation and recycling of cellular components.

Modulation (of gene expression): detectable or significant increase or decrease of gene expression in the presence of a compound or natural extract, whereby preferably a therapeutic, prophylactic, and/or cosmetic benefit is achieved. Gene expression modulation (i.e., increasing or decreasing expression) can be measured by fold change in gene expression, such as greater than about and fold increase/decrease, greater than about an 2 fold increase/decrease, greater than about an 5 fold increase/decrease, greater than about an 10 fold increase/decrease, and greater than about an 50 fold increase/decrease. Gene expression can be measured in a cell exposed to the compound or natural extract and is compared to the gene expression in a cell not exposed to the compound or natural extract. Fold increase/decrease can be determined based upon the measured gene expression levels. Modulation and other effects described herein typically are associated with a detectable or significant change as compared with a control or baseline condition. Any embodiment or term described herein in connection with one or more effects, such as modulation, will be understood to implicitly disclose a corresponding embodiment in which a significant effect is achieved as compared to control, baseline, or both. The occasional explicit reference to “significant” effects in parts of this disclosure does not impact this construction.

In some embodiments, the term “significantly” means resulting in a statistically significant effect, using an appropriate statistical test (e.g., p<0.1, p<0.05, or p<0.01, in a well designed, controlled study).

In an embodiment, the endocannabinoid mimetic composition of the invention comprises:
a) at least one direct endocannabinoid mimetic compound, wherein the at least one direct endocannabinoid mimetic compound detectably or significantly modulates (preferably increases) gene expression of the CB1 and/or CB2 gene, and is curcumin, B-caryophyllene, N-palmitoylethanolamide (PEA), ajulemic acid, 3,3-diindolylmethane (DIM), falcarinol, N-alkylamides, N-isobuytlamides, N-oleoylethanolamide, phenylpropanol, a salt of dehydroacetic acid, honokiol, magnolol, 7-hydroxyflavone, triptolide, ginkolide, docosahexaenoic acid (DHA), eicosapentaenoic acid (EPA), rutamarin, eugenol, menthol, camphor, methyl salicylate, disophenol, isomenthone, menthone, and limonene, salvinorin A, triterpene alcohols triterpendiol monoesters including faradiol esters, or any combination thereof;
b) at least one indirect endocannabinoid mimetic compound, wherein each such compound:
   1) detectably or significantly modulates (preferably decreases) gene expression of FAAH, in which case the compound is curcumin, tetrahydrocurcumin, B-caryophyllene, N-alkylamides, 7-hydroxyflavone, 3,7-dihydroxyflavone, daidzein, genestein, quercetin, kaempherol, pristimerin, phloretin, N-linoleoylethanolamide, N-oleoylethanolamide, N-acylethanolamines, N-palmitoylethanolamide, N-acetyl L-cysteine, sabinen, terpineol, a-pinene, limonene, terpinene, triptolide, ginsenoside, docosahexaenoic acid (DHA), eicosapentaenoic acid (EPA), ginkolide, eugenol, disophenol, isomenthone, menthone, limonene, honokiol, magnolol, methyl salicylate, camphor, menthol, or myristicin; and/or
   2) detectably or significantly modulates (preferably decreases) gene expression of MAGL, in which case the compound is pristimerin, B-caryophyllene, curcumin, N-oleoylethanolamide, N-palmitoylethanolamide, N-alkylamides, 7-hydroxyflavone, triptolide, ginsenoside, ginkolide, N-acetyl L-cysteine, diosphenol, isomenthone, menthone, limonene, docosahexaenoic acid (DHA), eicosapentaenoic acid (EPA), eugenol, honokiol, magnolol, methyl salicylate, camphor, menthol, myristicin, sabinen, terpineol, a-pinene, limonene, or terpinene;
c) at least one ECS related pathway anti-inflammatory compound, wherein each such compound:
   1) detectably or significantly modulates (preferably increases) gene expression of PPARgamma (PPARg), PPARalpha (PPARa), PPARbeta (PPARb), or any combination thereof; in which case the compound is ajulemic acid, B-caryophyllene, N-alkylamides, N-isobutylamides, apigenin, daidzein, genestein, quercetin, kaempherol, phloretin, N-acylethanolamines, N-palmitoylethanolamide, N-oleoylethanolamide, epigallocatechin gallate (EGCG), astaxanthin, beta carotene, lycopene, N-acetyl L-cysteine (NAC), diosphenol, isomenthone, menthone, limonene, rosmarinic acid, t-reservatrol, triptolide, myristicin, 7-hydroxyflavone, honokiol, magnolol, carvacrol, thymol, eugenol, docosahexaenoic acid (DHA), eicosapentaenoic acid (EPA), salicin, allicin, a-Lipoic acid, curcumin, ginkolide, methyl salicylate, camphor, menthol, ginsenoside, triterpene alcohols & triterpendiol monoesters (faradiol), 3,3-diindolylmethane (DIM), tetrahydrocurcurmin, cinnamaldehyde or capsaicin; and/or
   2) detectably or significantly modulates (preferably decreases) gene expression of COX1 (i.e., PTGS1), COX2, iNOS, 5-LOX, 12-LOX, MMP1, or any combination thereof, in which case the compound is curcumin, B-caryophyllene, N-alkylamides, N-palmitoylethanolamide, N-oleoylethanolamide, hyperforin, hypericin, epigallocatechin gallate, (−)a-bisabolol, astaxanthin, beta carotene, O-rhamnosylswertisin, a/b amyrenone, licochalcone A, alpha-lipoic acid, lycopene, N-acetyl L-cysteine, rosmarinic acid, perilloxin, perilla anthocyanin, t-reservatrol, verbascoside, echinoscoside, carnosine, pycnogenol, triptolide, a-pinene, actanol, linalool, octyl acetate, bornyl acetate, incensole, linalool, antizingiberol, zingiberene, phellandrene, gingerol, camphene, carvacrol, thymol, ginsenosides, sesqueterpene lactones, parthenolide, pentacyclic oxindole alkaloids, propofol, honokiol, magnolol, eugenol, diosphenol, isomenthone, menthone, limonene, ginkolide, 7-hydroxyflavone, docosahexaenoic acid (DHA), eicosapentaenoic acid (EPA), camphor, eucalyptol, camphene, β-pinene, borneol, thujone, sabinen, terpineol, a-pinene, limonene, terpinene, cinnamaldehyde, aescin, tetrahydrocurcurmin, triterpene alcohols & triterpendiol monoesters (faradiol), myristicin, allicin, apigenin, menthol, or methyl salicylate; and/or
   3) detectably or significantly modulates (preferably decreases) gene expression of IL-1beta, IL-1alpha(IL1a), IL-6, IL-8, NFKappaBeta (NFKB), TNFalpha (TNFa), and detectably or significantly modulates (preferably increases) gene expression of IL-10, or any combination thereof; in which case the compound is phenylpropanol, a salt of dehydroacetate, B-caryophyllene, N-palmitoylethanolamide, N-oleoylethanolamide, N-alkylamides, 7-hydroxyflavone, honokiol, magnolol, triptolide, N-acetyl L-cysteine, ginsenoside, ginkolide, diosphenol, isomenthone, menthone, limonene, triterpene alcohols and faradiol (Marigold extract), epigallocatechin gallate, apigenin, myristicin, O-rhamnosylswertisin, a/b amyrenone, rosmarinic acid, perilloxin, perilla anthocyanin, silymarin, verbascoside, echinoscoside, hyaluronic acid, a-pinene, actanol, linalool, octyl acetate, bornyl acetate, incensole, eugenol, curcumin, sesqueterpene lactones, parthenolide, pentacyclic oxindole alkaloids, docosahexaenoic acid (DHA), eicosapentaenoic acid (EPA), (−)a-bisabolol, licochalcone A, a-lipoic acid, lycopene, carnosine, hydrolyzed sodium hyaluronate, astaxanthin, B carotene, alpha-linolenic, acid, vitamin C, vitamin E, ferulic acid, chlorogenic acid, cafeic acid, quinic acid, olive polyphenols, capsanthin, carnosine, L-ergothioneine, 3,3-diindolylmethane (DIM), tetrahydrocurcurmin, t-resveratrol, carvacrol, thymol, allicin, camphor, eucalyptol, camphene, β-pinene, borneol, thujone, krill oil, fish oil, menthol, methyl salicylate, carvacrol, thymol, or linalool; and
d) at least one ECS related TRP pathway compound, wherein the at least one ECS related TRP pathway compound modulates gene expression of TRPA1, TRPM8, TRPV1, TRPV3, TRPV4, TRPV6, or any combination thereof, and is curcumin, trans-beta caryophyllene, alpha-linolenic acid, docosahexaenoic acid (DHA), eicosapentaenoic acid (EPA), ginkolide, a-pinene, actanol, linalool, octyl acetate, bornyl acetate, incensole, incensyl acetate, antizingiberol, zingiberene, phellandrene, gingerol, menthol, carvone, carvacrol, thymol, capsaicin, salicin, methyl salicylate, vanillic aldehyde, piperine, eugenol, ginsenosides, methyl chevicol, cinnamaldehyde, allyl isothiocyanate, sesqueterpene lactones, parthenolides triptolide, myristicin, diosphenol, isomenthone, menthone, limonene, N-palmitoylethanolamide, N-oleoylethanolamide, N-alkylamides, honokiol, camphor, eucalyptol, camphene, β-pinene, borneol, thujone, aubergine, chelerythrine & magnoflorine, sabinen, terpineol, limonene, terpinene, carnosic acid, cineol, viridiflorol, terpineol, pinene, limonene, 7-hydroxyflavone, 3,7-dihydroxyflavone, allicin, or any combination thereof; and
   wherein gene expression in each case is measured in a cell exposed to the compound and is compared to the gene expression in a cell not exposed to the compound.

In another embodiment, the endocannabinoid mimetic composition of the invention comprises:
a) at least one direct endocannabinoid mimetic compound, wherein the at least one direct endocannabinoid mimetic compound detectably or significantly modulates (preferably increases) gene expression of the CB1 and/or CB2 gene, and is curcumin, B-caryophyllene, N-palmitoylethanolamide, ajulemic acid, 3,3-diindolylmethane (DIM), falcarinol, N-alkylamides, N-isobuytlamides, N-oleoylethanolamide, phenylpropanol, a salt of dehydroacetic acid, salvinorin A, honokiol, magnolol, 7-hydroxyflavone, triptolide, ginkolide, pentacyclic triterpene alcohols and triterpendiol monoesters including faradiol esters, diosphenol docosahexaenoic acid (DHA), eicosapentaenoic acid (EPA), rutamarin, eugenol, or any combination thereof;
b) at least one indirect endocannabinoid mimetic compound, wherein each compound:
   1) detectably or significantly modulates (preferably decreases) gene expression of FAAH, in which case the compound curcumin, B-caryophyllene, N-alkylamides, 7-hydroxyflavone, 3,7-dihydroxyflavone, apigenin, daidzein, genestein, quercetin, kaempherol, pristimerin, phloretin, N-linoleoylethanolamide, N-oleoylethanolamide, N-acylethanolamines, N-palmitoylethanolamide, N-acetyl L-cysteine, sabinen, terpineol, a-pinene, triptolide, ginsenoside, ginkolide, docosahexaenoic acid (DHA), eicosapentaenoic acid (EPA), ginkolide, or eugenol; and/or
   2) detectably or significantly modulates (preferably decreases) gene expression of MAGL, in which case the compound pristimerin, B-caryophyllene, curcumin, N-oleoylethanolamide, N-palmitoylethanolamide, N-alkylamides, 7-hydroxyflavone, triptolide, ginsenoside, ginkolide, N-acetyl L-cysteine, diosphenol, docosahexaenoic acid (DHA), eicosapentaenoic acid (EPA), or eugenol;
c) at least one ECS related pathway anti-inflammatory compound, wherein each such compound:
   1) detectably or significantly modulates (preferably increases) gene expression of PPARgamma (PPARg), PPARalpha (PPARa), PPARbeta (PPARb), or any combination thereof; in which case the compound is ajulemic acid, B-caryophyllene, N-alkylamides, N-isobutylamides, apigenin, daidzein, genestein, quercetin, kaempherol, phloretin, N-acylethanolamines, N-palmitoylethanolamide, N-oleoylethanolamide, epigallocatechin gallate, astaxanthin, beta carotene, beta-glucan, lycopene, N-acetyl L-cysteine, diosphenol, isomenthone, menthone, limonene, rosmarinic acid, t-reservatrol, triptolide, myristicin, 7-hydroxyflavone, honokiol, carvacrol, thymol, capsaicin, eugenol, docosahexaenoic acid (DHA), eicosapentaenoic acid (EPA), salicin, allicin, or a-lipoic acid; and/or
   2) detectably or significantly modulates (preferably decreases) gene expression of COX1 (i.e., PTGS1), COX2, iNOS, 5-LOX, 12-LOX, MMP1, or any combination thereof, in which case the compound is curcumin, B-caryophyllene, N-alkylamides, N-palmitoylethanolamide, N-oleoylethanolamide, hyperforin, hypericin, epigallocatechin gallate, (−)a-bisabolol, astaxanthin, beta carotene, beta-glucan, 0-rhamnosylswertisin, a/b amyrenone, licochalcone A, alpha-lipoic acid, lycopene, N-acetyl L-cysteine, rosmarinic acid, perilloxin, perilla anthocyanin, t-reservatrol, verbascoside, echinoscoside, carnosine, pycnogenol, hyaluronic acid, triptolide, a-pinene, actanol, linalool, octyl acetate, bornyl acetate, incensole, linalool, antizingiberol, zingiberene, phellandrene, gingerol, camphene, carvacrol, thymol, ginsenosides, sesqueterpene lactones, parthenolide, pentacyclic oxindole alkaloids, propofol, honokiol, magnolol, eugenol, diosphenol, ginkolide, 7-hydroxyflavone, docosahexaenoic acid (DHA), eicosapentaenoic acid (EPA), camphor, eucalyptol, camphene, β-pinene, borneol, thujone, sabinen, terpineol, a-pinene, limonene, terpinene, cinnamaldehyde, or aescin; and/or
   3) detectably or significantly modulates (preferably decreases) gene expression of IL-1beta, IL-1alpha(IL1a), IL-6, IL-8, NFKappaBeta (NFKB), TNFalpha (TNFa), and detectably or significantly modulates (preferably increases) gene expression of IL-10, or any combination thereof; in which case the compound is phenylpropanol, a salt of dehydroacetate, B-caryophyllene, N-palmitoylethanolamide, N-oleoylethanolamide, N-alkylamides, 7-hydroxyflavone, honokiol, magnolol, triptolide, N-acetyl L-cysteine, ginsenoside, ginkolide, diosphenol, isomenthone, menthone, limonene, triterpene alcohols and faradiol (Marigold extract), epigallocatechin gallate, apigenin, myristicin, O-rhamnosylswertisin, a/b amyrenone, rosmarinic acid, perilloxin, perilla anthocyanin, silymarin, verbascoside, echinoscoside, hyaluronic acid, a-pinene, actanol, linalool, octyl acetate, bornyl acetate, incensole, eugenol, curcumin, sesqueterpene lactones, parthenolide, pentacyclic oxindole alkaloids, docosahexaenoic acid (DHA), eicosapentaenoic acid (EPA), (−)a-bisabolol, licochalcone A, a-lipoic Acid, lycopene, carnosine, hydrolyzed sodium hyaluronate, astaxanthin, B carotene, alpha-linolenic, acid, vitamin C, vitamin E, ferulic acid, chlorogenic acid, cafeic acid, quinic acid, olive polyphenols, capsanthin, carnosine, or L-ergothioneine; and
d) at least one ECS related TRP pathway compound, wherein the at least one ECS related TRP pathway compound modulates gene expression of TRPA1, TRPM8, TRPV1, TRPV3, TRPV4, TRPV6, or any combination thereof, and is curcumin, trans-beta caryophyllene, alpha-linolenic acid, docosahexaenoic acid (DHA), eicosapentaenoic acid (EPA), ginkolide, a-pinene, actanol, linalool, octyl acetate, bornyl acetate, incensole, linalool, antizingiberol, zingiberene, phellandrene, gingerol, camphene, eucalyptol, menthol, carvone, carvacrol, thymol, capsaicin, salicin, methyl salicylate, vanillic aldehyde, piperine, eugenol, ginsenosides, mentyl PCA, camphor, allicin thiosulfinates, methyl chevicol, cinnamaldehyde, allyl isothiocyanate, sesqueterpene lactones, parthenolides triptolide, myristicin, diosphenol; N-palmitoylethanolamide, N-oleoylethanolamide, N-alkylamides, honokiol, camphor, eucalyptol, camphene, β-pinene, borneol, thujone, linalool, antizingiberol, zingiberene, phellandrene, gingerol, camphene, carvone, linalool, aubergine, chelerythrine & magnoflorine, sabinen, terpineol, a-pinene, limonene, terpinene, carnosic acid, cineol, viridiflorol, terpineol, pinene, limonene, (−)a-bisabolol, ginkolide, or any combination thereof; and
   wherein gene expression in each case is measured in a cell exposed to the compound and is compared to the gene expression in a cell not exposed to the compound.

Certain embodiments may contain chemical classes of compounds that include a specification based on a common molecular structure relationship that impacts the functional utility for the gene modulation purpose, such classes and chemical structure relationships are defined hereunder.

Curcuminoids: linear diarylheptanoids that include two aromatic rings (aryl or phenyl groups) joined by a seven carbon chain (heptane).
Allyl Chain Substituted Guaiacols: methoxy phenolic compounds [C₆H₄(OH)(OCH₃)] that have an additional allyl group attachment.
Fatty Acid Amides: compounds resulting from the combination of a fatty acid (long aliphatic chain carboxylic acid) and an amine, in many cases ethanolamine, that include the functionality RC(O)N(H)CH₂CH₂OH; but in some cases they may be primary amides that include the functionality RC(O)NH₂.
Ginsenosides: also referred to as panaxosides include steroid glycosides, wherein a sugar is attached to a steroid structure, and wherein the steroid structure is a triterpene, the resulting structure commonly referred to as a triterpene saponin (i.e. triterpene glycoside) derived from the plant genus Panax.
Monoterpenes: a class of terpenes that include of 2 isoprene [(2-methyl-1,3-butadiene)] units including monoterpenoids i.e. modified monoterpenes, such as those containing oxygen functionality or missing a methyl group.
Biphenols: compounds that have two phenolic hydroxy groups including stilbenoids.
Sesquiterpenes: a class of terpenes that include three isoprene units; including sesquiterpenoids or modified sesquiterpenes in a wide variety of forms, including linear, monocyclic, bicyclic, and tricyclic frameworks.
Terpene Lactones: modified diterpenes (diterpenoids) or sesquiterpenes (sesquiterpenoids) that contain a lactone ring.
Flavan-3-ols: any flavans that possess a 2-phenyl-3,4-dihydro-2H-chromen-3-ol skeleton
Hydroxyflavones: a class of flavonoids with one or more hydroxy groups on a flavone backbone
Diterpenes: a class of terpenes include four isoprene units including modified diterpenes or diterpenoids.
Triterpenes: a class of terpenes include of six isoprene units, and triterpenoids that originate from squalene via condensation reactions.
N-alkylamides: amides possessing an aromatic or aliphatic unsaturated fatty acid residue linked to an aliphatic or aromatic amine residue, including isobutylamides.
PUFAs: Polyunsaturated fatty acids are fatty acids that contain more than one double bond in their backbone

The endocannabinoid mimetic compounds of the present invention include compounds that directly affect the functioning of the endocannabinoid system by, e.g., increasing the expression of a Cannabinoid Receptor Type 1, CNR1 (CB1) gene or a Cannabinoid Receptor Type 2, CNR2 (CB2) gene. See FIG. 2 depicting a non-limiting schematic of the endocannabinoid system.

Endocannabinoid mimetic compounds that increase CB1 or CB2 gene expression include, but are not limited to, curcumin, B-caryophyllene, N-palmitoylethanolamide, ajulemic acid, 3,3-diindolylmethane (DIM), falcarinol, N-alkylamides, N-isobuytlamides, N-oleoylethanolamide, phenylpropanol, a salt of dehydroacetic acid, salvinorin A, honokiol, magnolol, 7-hydroxyflavone, triptolide, ginkolide, pentacyclic triterpene alcohols and triterpendiol monoesters including faradiol esters, diosphenol isomenthone, menthone, limonene, docosahexaenoic acid (DHA), eicosapentaenoic acid (EPA), rutamarin, eugenol, and any combination thereof.

In an another embodiment, compounds for increasing CB1 or CB2 gene expression include curcumin, B-caryophyllene, N-palmitoylethanolamide, 3,3-diindolylmethane (DIM), N-alkylamides, phenylpropanol, a salt of dehydroacetic acid, salvinorin A, honokiol, magnolol, triptolide, ginkolide, triterpene alcohols and triterpendiol monoesters including faradiol esters, rutamarin, eugenol, and any combination thereof.

In another embodiment, compounds for increasing CB1 or CB2 gene expression include curcumin, B-caryophyllene, N-palmitoylethanolamide, N-alkylamides, honokiol, magnolol, triptolide, ginkolide, eugenol, and any combination thereof.

In another embodiment, compounds for increasing CB1 gene expression include curcumin, N-palmitoylethanolamide, honokiol, magnolol, ginkolide, eugenol, and any combination thereof.

In another embodiment, compounds for increasing CB2 gene expression include curcumin, B-caryophyllene, N-palmitoylethanolamide, N-alkylamides, honokiol, magnolol, triptolide, eugenol, and any combination thereof.

In an another embodiment, compounds for increasing CB1 gene expression include curcumin, B-caryophyllene, N-palmitoylethanolamide, N-oleoylethanolamide, N-alkylamides, honokiol, 7-hydroxyflavone, triptolide, ginkolide, docosahexaenoic acid (DHA), eicosapentaenoic acid (EPA), diosphenol, isomenthone, menthone, limonene, eugenol, and any combination thereof.

In an another embodiment, compounds for increasing CB2 gene expression include curcumin, B-caryophyllene, N-palmitoylethanolamide, N-oleoylethanolamide, N-alkylamides, honokiol, magnolol, 7-hydroxyflavone, triptolide, docosahexaenoic acid (DHA), eicosapentaenoic acid (EPA), diosphenol, isomenthone, menthone, limonene, eugenol, and any combination thereof.

Further, compounds affecting CB1 and CB2 gene expression can be classified by chemical class. In such embodiment, the chemical classes for modulating CB1 and/or CB2 gene expression include curcuminoids, and allyl chain substituted guaiacols.

In another embodiment, the curcuminoid chemical class includes, but is not limited to, curcumin, demethoxycurcumin, bisdemethoxycurcumin, and tetrahydrocurcumin.

In another embodiment the allyl chain substituted guaiacols chemical class includes, but is not limited to, eugenol, its isomers, and its derivatives including isoeugenol, dihydroeugenol, and ethyl guaiacol.

Other endocannabinoid mimetic compounds of the present invention indirectly affect the functioning of the endocannabinoid system. Without wishing to be bound by theory, it is believed that the compounds affect the endocannbinoid system by influencing the metabolism (anabolism or catabolism) of the endogenous ligands, anandamide (AEA) or 2-arachidonoylglycerol (2-AG), which in turn interact with CB1 and/or CB2. Enzymes involved in the degradation (catabolism) of AEA include fatty acid amide hydrolase (FAAH), and the synthesis (anabolism) involves N-acyl phosphatidylethanolamine-specific phospholipase D (NAPE-PLD). Enzymes involved in the degradation of 2AG include monacylglycerol lipase (MAGL), those involved in the synthesis include diacylglycerol lipase 1 and 2 (DAGL1 and DAGL2). Accordingly, certain endocannabinoid mimetic compounds are thought to indirectly affect the functioning of the endocannabinoid system by, e.g., increasing or decreasing the expression of at least one of the following genes: FAAH, NAPE-PLD, MAGL, DAGL1, DAGL2. As shown in FIG. 2 there is a metabolic pathway associated with each endocannabinoid, i.e., there is an anandamide and a 2-arachidonoylglycerol metabolic pathway. The former pathway includes NAPE-PLD and FAAH, while the latter pathway includes MAGL, DAGL1, and DAGL2.

Endocannabinoid mimetic compounds that decrease FAAH gene expression include, but are not limited to, curcumin, B-caryophyllene, N-alkylamides, 7-hydroxyflavone, 3,7-dihydroxyflavone, apigenin, daidzein, genestein, quercetin, kaempherol, pristimerin, phloretin, N-linoleoylethanolamide, N-oleoylethanolamide, N-acylethanolamines, N-palmitoylethanolamide, N-acetyl L-cysteine, sabinen, terpineol, a-pinene, limonene, terpinene, triptolide, ginsenoside, ginkolide, docosahexaenoic acid (DHA), eicosapentaenoic acid (EPA), eugenol, tetrahydrocurcurmin, and any combination thereof.

In an another embodiment, compounds for decreasing FAAH gene expression include curcumin, B-caryophyllene, N-alkylamides, 7-hydroxyflavone, 3,7-dihydroxyflavone, daidzein, genestein, quercetin, kaempherol, phloretin, N-oleoylethanolamide, N-palmitoylethanolamide, N-acetyl L-cysteine, sabinen, terpineol, a-pinene, limonene, terpinene, triptolide, ginsenoside, ginkolide, eugenol, tetrahydrocurcurmin, and any combination thereof.

In an another embodiment, compounds for decreasing FAAH gene expression include B-caryophyllene, curcumin, N-oleoylethanolamide, N-palmitoylethanolamide, N-alkylamides, 7-hydroxyflavone, triptolide, ginsenoside, ginkolide, N-acetyl L-cysteine, eugenol, and any combination thereof.

In an another embodiment, compounds for decreasing FAAH gene expression include B-caryophyllene, curcumin, N-oleoylethanolamide, N-palmitoylethanolamide, N-alkylamides, 7-hydroxyflavone, docosahexaenoic acid (DHA), eicosapentaenoic acid (EPA), triptolide, ginsenoside, ginkolide, N-acetyl L-cysteine, eugenol, and any combination thereof.

In one embodiment, the endocannabinoid mimetic compounds that decrease MAGL gene expression include pristimerin, B-caryophyllene, curcumin, N-oleoylethanolamide, N-palmitoylethanolamide, N-alkylamides, 7-hydroxyflavone, triptolide, ginsenoside, ginkolide, N-acetyl L-cysteine, diosphenol, isomenthone, menthone, limonene, docosahexaenoic acid (DHA), eicosapentaenoic acid (EPA), eugenol, sabinen, terpineol, a-pinene, limonene, terpinene, and any combination thereof.

In an another embodiment, compounds for decreasing MAGL gene expression include pristimerin, B-caryophyllene, curcumin, N-alkylamides, 7-hydroxyflavone, triptolide, ginsenoside, ginkolide, N-acetyl L-cysteine, eugenol, sabinen, terpineol, a-pinene, limonene, terpinene, and any combination thereof.

In another embodiment, compounds for decreasing MAGL gene expression include pristimerin, curcumin, triptolide, ginsenoside, N-acetyl L-cysteine, sabinen, terpineol, a-pinene, limonene, terpinene, eugenol, and any combination thereof.

In an another embodiment, compounds for decreasing MAGL gene expression include B-caryophyllene, curcumin, N-oleoylethanolamide, N-palmitoylethanolamide, N-alkylamides, 7-hydroxyflavone, docosahexaenoic acid (DHA), eicosapentaenoic acid (EPA), triptolide, ginsenoside, N-acetyl L-cysteine, diosphenol, isomenthone, menthone, limonene, eugenol, and any combination thereof.

Further, compounds affecting FAAH and/or MAGL gene expression can be classified by chemical class. In such embodiment, the chemical classes for modulating FAAH and/or MAGL gene expression include fatty acid amides and ginsenosides.

The fatty acid amide chemical class includes, but is not limited to, N-oleoylethanolamide (OEA), N-palmitoylethanolamide (PEA), N-linoleoylethanolamide, N-acylethanolamines, Stearoylethanolamide (SEA), Oleamide, and Arachidonamide.

The ginsenoside chemical class includes, but is not limited to, compounds derived from plants of the plant genus Panax (Ginseng), including ginsenoside RC.

The ECS related pathway anti-inflammatory compounds of the present invention influence inflammatory processes in various fashions. Some anti-inflammatory compounds affect a nuclear pathway that increases expression of PPARg, PPARa, PPARb, and any combination thereof.

In one embodiment, the endocannabinoid mimetic compounds that increase PPARg gene expression include ajulemic acid, B-caryophyllene, N-alkylamides, N-isobutylamides, apigenin, daidzein, genestein, quercetin, kaempherol, phloretin, N-acylethanolamines, N-palmitoylethanolamide, N-oleoylethanolamide, epigallocatechin gallate, astaxanthin, beta carotene, lycopene, N-acetyl L-cysteine, diosphenol, isomenthone, menthone, limonene, rosmarinic acid, t-reservatrol, triptolide, myristicin, 7-hydroxyflavone, honokiol, carvacrol, thymol, capsaicin, eugenol, docosahexaenoic acid (DHA), eicosapentaenoic acid (EPA), salicin, allicin, alpha-Lipoic Acid (a-Lipoic Acid), and any combination thereof.

In an another embodiment, compounds for increasing PPARg gene expression include daidzein, genestein, quercetin, kaempherol, phloretin, N-palmitoylethanolamide, N-oleoylethanolamide, epigallocatechin gallate, astaxanthin, beta carotene, N-acetyl L-cysteine, diosphenol, isomenthone, menthone, limonene, rosmarinic acid, t-reservatrol, triptolide, myristicin, 7-hydroxyflavone, honokiol, carvacrol, thymol, eugenol, docosahexaenoic acid (DHA), eicosapentaenoic acid (EPA), salicin, allicin, a-Lipoic Acid, lycopene, and any combination thereof.

In an another embodiment, compounds for increasing PPARg gene expression include N-palmitoylethanolamide, N-oleoylethanolamide, diosphenol, isomenthone, menthone, limonene, triptolide, myristicin, 7-hydroxyflavone, honokiol, eugenol, docosahexaenoic acid (DHA), eicosapentaenoic acid (EPA), epigallocatechin gallate, N-acetyl L-cysteine, and any combination thereof.

In an another embodiment, compounds for increasing PPARg gene expression include N-palmitoylethanolamide, N-oleoylethanolamide, 7-hydroxyflavone, honokiol, diosphenol, isomenthone, menthone, limonene, docosahexaenoic acid (DHA), eicosapentaenoic acid (EPA), triptolide, myristicin, eugenol, and any combination thereof.

Further, compounds effecting PPARg gene expression can be classified by chemical class. In such embodiment, the chemical classes for modulating PPARg gene expression include monoterpenes and biphenols.

The monoterpenes chemical class includes, but is not limited to, diosphenol, isomenthone, menthone, limonene, menthol, myrcene, linalool, pinene, and camphor.

In another embodiment the biphenol chemical class includes, but is not limited to, honokiol, magnolol, and stilbenoids such as resveratrol and diethylstilbestrol.

Other endocannabinoid mimetic anti-inflammatory compounds suitable for inclusion in the invention affect an enzymatic pathway that decreases expression of COX1 (i.e., PTGS1), COX2, iNOS, 5-LOX, 12-LOX, MMP1, or any combination thereof. Such compounds include, but are not limited to, curcumin, B-caryophyllene, N-alkylamides, N-palmitoylethanolamide, N-oleoylethanolamide, hyperforin, hypericin, epigallocatechin gallate, (−)a-bisabolol, astaxanthin, beta carotene, O-rhamnosylswertisin, a/b amyrenone, licochalcone A, alpha-lipoic acid, lycopene, N-acetyl L-cysteine, rosmarinic acid, perilloxin, perilla anthocyanin, t-reservatrol, verbascoside, echinoscoside, carnosine, pycnogenol, triptolide, a-pinene, actanol, linalool, octyl acetate, bornyl acetate, incensole, incensyl acetate, linalool, antizingiberol, zingiberene, phellandrene, gingerol, camphene, carvacrol, thymol, ginsenosides, sesqueterpene lactones, parthenolide, pentacyclic oxindole alkaloids, honokiol, magnolol, eugenol, diosphenol, isomenthone, menthone, limonene, ginkolide, 7-hydroxyflavone, docosahexaenoic acid (DHA), eicosapentaenoic acid (EPA), camphor, eucalyptol, camphene, β-pinene, borneol, thujone, sabinen, terpineol, a-pinene, limonene, terpinene, cinnamaldehyde, aescin, and tetrahydrocurcurmin.

Compounds that decrease expression of COX1 (i.e, PTGS1), COX2, iNOS, 5-LOX, 12-LOX, MMP1, or any combination thereof; include curcumin, epigallocatechin gallate, N-acetyl L-cysteine, rosmarinic acid, perilloxin, perilla anthocyanin, verbascoside, echinoscoside, pycnogenol, triptolide, a-pinene, actanol, linalool, octyl acetate, bornyl acetate, incensole, incensole acetate, linalool, antizingiberol, zingiberene, phellandrene, gingerol, camphene, carvacrol, thymol, ginsenosides, sesqueterpene lactones, parthenolide eugenol, ginkolide, camphor, eucalyptol, camphene, β-pinene, borneol, thujone, sabinen, terpineol, a-pinene, limonene, terpinene, actanol cinnamaldehyde, aescin, tetrahydrocurcurmin, and any combination thereof.

Further compounds that decrease expression of COX1 (i.e, PTGS1), COX2, iNOS, 5-LOX, 12-LOX, MMP1, or any combination thereof are curcumin, B-caryophyllene, triptolide, ginsenosides, ginkolide, eugenol, epigallocatechin gallate, and any combination thereof.

Other compounds that decrease expression of COX1 (i.e, PTGS1), COX2, iNOS, 5-LOX, 12-LOX, MMP1, or any combination thereof, are curcumin, B-caryophyllene, N-palmitoylethanolamide, N-oleoylethanolamide, N-alkylamides 7-hydroxyflavone, docosahexaenoic acid (DHA), eicosapentaenoic acid (EPA), triptolide, ginsenoside, diosphenol, isomenthone, menthone, limonene, ginkolide, eugenol, and any combination thereof.

Further, compounds effecting PTGS1 gene expression can be classified by chemical class. In such embodiment, the chemical classes for modulating PTGS1 gene expression include sesquiterpenes, terpene lactones, and flavan-3-ols.

The sesquiterpenes chemical class includes, but is not limited to, β caryophyllene, humulene, farnesenes, farnesol, zingiberene, longifolene, copaene, and patchoulol.

The terpene lactones chemical class includes, but is not limited to, ginkolide A, ginkolide B, ginkolide C, ginkolide J, ginkolide M, bilobalide, parthenolide, helenalin, lactucin, and lactucopicrin.

The flavan-3-ol chemical class includes, but is not limited to, epigallocatechin gallate, catechin, epicatechin, gallocatechin, epigallocatechin, catechin gallate, and epicatechin gallate.

Still other ECS related pathway anti-inflammatory compounds suitable for inclusion in the invention affect a cytokine pathway that decrease expression of IL-1beta, IL-1alpha, IL-6, IL-8, NFKB and TNFalpha, increase gene expression in IL-10, or any combination thereof, include phenylpropanol, a salt of dehydroacetate, B-caryophyllene, N-palmitoylethanolamide, N-oleoylethanolamide, N-alkylamides, 7-hydroxyflavone, honokiol, magnolol, triptolide, N-acetyl L-cysteine, ginsenoside, ginkolide, diosphenol, isomenthone, menthone, limonene, triterpene alcohols and faradiol (Marigold extract), epigallocatechin gallate, apigenin, myristicin, O-rhamnosylswertisin, a/b amyrenone, rosmarinic acid, perilloxin, perilla anthocyanin, silymarin, verbascoside, echinoscoside, a-pinene, actanol, linalool, octyl acetate, bornyl acetate, incensole, incensyl acetate, eugenol, curcumin, sesqueterpene lactones, parthenolide, pentacyclic oxindole alkaloids, docosahexaenoic acid (DHA), eicosapentaenoic acid (EPA), (−)a-bisabolol, licochalcone A, a-Lipoic acid, lycopene, carnosine, hydrolyzed sodium hyaluronate, astaxanthin, b carotene, alpha-linolenic acid, carnosine, tetrahydrocurcurmin, and any combination thereof.

Compounds that decrease expression of IL-1beta, IL-1alpha, IL-6, IL-8, NFKB and TNFalpha, increase gene expression in IL-10, or any combination thereof, include phenylpropanol, a salt of dehydroacetate, B-caryophyllene, N-palmitoylethanolamide, N-oleoylethanolamide, N-alkylamides, 7-hydroxyflavone, honokiol, magnolol, triptolide, N-acetyl L-cysteine, ginsenoside, ginkolide, diosphenol, isomenthone, menthone, limonene, triterpene alcohols and faradiol, epigallocatechin gallate, apigenin, myristicin, O-rhamnosylswertisin, a/b amyrenone, perilloxin, perilla anthocyanin, silymarin, verbascoside, echinoscoside, a-pinene, actanol, linalool, octyl acetate, bornyl acetate, incensole, incensyl acetate, curcumin, sesqueterpene lactones, parthenolide, pentacyclic oxindole alkaloids, docosahexaenoic acid (DHA), eicosapentaenoic acid (EPA), (−)a-bisabolol, licochalcone A, a-lipoic acid, lycopene, carnosine, hydrolyzed sodium hyaluronate, astaxanthin, b carotene rosmarinic acid, tetrahydrocurcurmin, and any combination thereof.

Further compounds that decrease IL-1beta, IL-1alpha, IL-6, IL-8, NFKB and TNFa, increase gene expression in IL-10, or any combination thereof, include diosphenol, isomenthone, menthone, limonene, epigallocatechin gallate, apigenin, N-acetyl L-cysteine, curcumin, B-caryophyllene, N-palmitoylethanolamide, N-oleoylethanolaminde, honokiol, magnolol, 7-hydroxyflavone, docosahexaenoic acid (DHA), eicosapentaenoic acid (EPA), triptolide, ginsenosides, ginkolide, myristicin, N-alkylamides, and triterpene alcohols faradiol, and any combination thereof.

Compounds that decrease gene expression in IL-1a include curcumin, B-caryophyllene, N-palmitoylethanolamide, N-oleoylethanolamide, honokiol, magnolol, 7-hydroxyflavone, docosahexaenoic acid (DHA), eicosapentaenoic acid (EPA), triptolide, ginsenoside, ginkolide, diosphenol, isomenthone, menthone, limonene, myristicin, epigallocatechin gallate, apigenin, and any combination thereof.

Compounds that decrease gene expression in NFKB include curcumin, B-caryophyllene, N-palmitoylethanolamide, N-oleoylethanolamide, N-alkylamides, 7-hydroxyflavone, triptolide, N-acetyl L-cysteine, docosahexaenoic acid (DHA) eicosapentaenoic acid (EPA), ginkolide, diosphenol, isomenthone, menthone, limonene, epigallocatechin gallate, triterpene alcohols, faradiol, and any combination thereof.

Compounds affecting NFKB and IL1a gene expression can be classified by chemical class. In such embodiment, the chemical classes for modulating NFKB and IL1a gene expression include hydroxyflavones, diterpenes, triterpenes and N-acetyl L-cysteine.

The hydroxyflavones chemical class includes, but is not limited to, 7-hydroxyflavone, 3,7-dihydroxyflavone, myricetin, quercetin, fisetin, apigenin, and kaempferol.

The diterpenes chemical class includes, but is not limited to, triptolide, rosmanol, carnosic acid, salvinorin A, and forskolin.

The triterpenes chemical class includes, but is not limited to, triterpene alcohols & triterpendiol monoesters (such as faradiol).

Many of the compounds that affect the nuclear, enzymatic and cytokine inflammatory pathways listed herein are antioxidants. Other antioxidants also suitable for inclusion in the invention could include any antioxidant from any class of antioxidant listed below or any natural extract known to contain one or more of such anti-inflammatory antioxidant compounds:
Amino acid antioxidants: (e.g. tyrosine, cysteine and homocysteine)
Terpenes and derivatives: (e.g. citronellol)
Respiratory chain antioxidants and derivatives: (e.g. superoxide dismutase, Co-enzyme Q10, catalase, idebenone, PQQ),
Polyphenols and derivatives: (e.g. olive polyphenols)
Sulphur based endogenous antioxidants: (e.g. glutathione)
stilbenoids (e.g. piceatannol, pterostilbene, and astringin),
curcumininoids (e.g. demethoxycurcumin, and bisdemethoxycurcumin),
tannins (e.g. gallic acid, gallic acid C₁₋₁₂ alkyl esters, ethyl gallate, propyl gallate, octyl gallate, dodecyl gallate, theaflavin esters of gallic acid, and condensed tannins/e.g., proanthocyanidins, prodelphinidins, procyanidins, oligomeric proanthocyanidins, leukocyanidins, leucoanthocyanins),
flavones (e.g. luteolin, tangeritin, chrysin, 6-hydroxyflavone, baicalein, scutellarein, wogonin, and orientin),
flavanols (e.g. 3-hydroxyflavone, azaleatin, fisetin, galangin, gossypetin, isorhamnetin, kaempferide, kaempferol, morin, myricetin, natsudaidain, pachypodol, rhamnazin, and rhamnetin),
flavan-3-ols (e.g. catechin, epicatechin, gallocatechin, epigallocatechin, catechin gallate, epicatechin gallate, epiafzelechin, fisetinidol, guibourtinidol, mesquitol, robinetinidol),
flavanones (e.g. butin, eriodictyol, hesperetin, hesperidin, homoeriodictyol, isosakuranetin, naringenin, pinocembrin, poncirin, sakuranetin, sakuranin, and sterubin),
anthocyanidins (e.g. aurantinidin, cyaniding, delphinidin, europinidin, luteolinidin, pelargonidin, malvidin, peonidin, petunidin, and rosinidin), anthocyanins,
isoflavones (e.g. iochanin, coumestrol, and formononetin)
flavanonols (e.g. taxifolin and aromadedrin),
diydroxybenzoic acids (e.g. protocatechuic acid)
pyridine alkaloids (e.g. trigonelline, arecoline, ricinine, actinidine, gentianine, and gentialutine), all of which may be sourced from natural extracts

In embodiments, the endocannabinoid mimetic direct compound, the endocannabinoid indirect compound, and the ECS related pathway anti-inflammatory compound-containing compositions of the invention further comprise at least one ECS related TRP pathway compound. The ECS related TRP pathways can have pain reducing and cell modulating effects and/or impact one or more of skin barrier function, cellular proliferation, differentiation, autophagy, apoptosis, and senescence. In some embodiments, the ECS related TRP pathway compounds preferably decrease gene expression involved in a pain-associated molecular pathway. In other embodiments, ECS related TRP pathway compounds may increase gene expression in a cellular modulation pathway. Preferred genes involved in a pain-associated molecular pathway include genes in the transient receptor potential cation channel, subfamily A, member 1 (TRPA) subfamily, including TRPA1, the transient receptor potential cation channel, melastatin (TRPM) subfamily, including TRPM8, the transient receptor potential cation channel, vanilloid (TRPV) subfamily, including TRPV1, TRPV3, TRPV4, and TRPV6. Still other anti-inflammatory compounds which are polyunsaturated fatty acids (PUFAs) or N-Alkylamides are suitable for inclusion in the invention modulate expression of TRPV1, TRPA1, and/or TRPM8 genes. In certain embodiments, the at least one polyunsaturated fatty acid compound that modulates expression of TRPV1, TRPA1, and/or TRPM8 genes, is alpha-linolenic acid (ALA), docosahexaenoic acid (DHA), eicosapentaenoic acid (EPA), or any combination thereof. In certain embodiments, the at least one N-Alkylamide compound that modulates expression of TRPV1, TRPA1, and/or TRPM8 genes is dodeca-2E,4E,8Z,10Z-tetraenoic acid isobutylamide, and/or dodeca-2E,4E-dienoic acid isobutylamide.

ECS related TRP pathway compounds that increase expression of TRPA1 or TRPM8, decrease gene expression for TRPV1, or any combination thereof, include curcumin, trans-beta caryophyllene, alpha-linolenic acid, docosahexaenoic acid (DHA), eicosapentaenoic acid (EPA), ginkolide, a-pinene, actanol, linalool, octyl acetate, bornyl acetate, incensole, incensyl acetate, linalool, antizingiberol, zingiberene, phellandrene, gingerol, camphene, eucalyptol, menthol, carvone, carvacrol, thymol, capsaicin, salicin, methyl salicylate, vanillic aldehyde, piperine, eugenol, ginsenosides, camphor, allicin thiosulfinates, methyl chevicol, cinnamaldehyde, allyl isothiocyanate, sesqueterpene lactones, parthenolides triptolide, myristicin, diosphenol, isomenthone, menthone, limonene, N-palmitoylethanolamide, N-oleoylethanolamide, N-alkylamides, honokiol, camphor, eucalyptol, camphene, β-pinene, borneol, thujone, carvone, linalool, aubergine, chelerythrine, magnoflorine, sabinen, terpineol, a-pinene, limonene, terpinene, carnosic acid, cineol, viridiflorol, terpineol, pinene, limonene, (−)a-bisabolol, tetrahydrocurcurmin, and any combination thereof.

In one embodiment, ECS related TRP pathway compounds that increase gene expression of TRPA1, or TRPM8, decrease gene expression of TRPV1, or any combination thereof, include curcumin, trans-beta caryophyllene, docosahexaenoic acid (DHA), eicosapentaenoic acid (EPA), ginkolide, menthol, carvone, carvacrol, thymol, capsaicin, salicin, methyl salicylate, vanillic aldehyde, piperine, eugenol, ginsenosides, methyl chevicol, cinnamaldehyde, allyl isothiocyanate, sesqueterpene lactones, parthenolides triptolide, myristicin, diosphenol, isomenthone, menthone, limonene, N-palmitoylethanolamide, N-oleoylethanolamide, N-alkylamides, honokiol, camphor, eucalyptol, camphene, β-pinene, borneol, thujone, linalool, antizingiberol, zingiberene, phellandrene, gingerol, camphene, linalool, aubergine, chelerythrine, magnoflorine, sabinen, terpineol, a-pinene, limonene, terpinene, a-pinene, actanol, linalool, octyl acetate, bornyl acetate, incensole, incensyl acetate, carnosic acid, cineol, viridiflorol, terpineol, pinene, limonene, tetrahydrocurcurmin, and any combination thereof.

In another embodiment, ECS related TRP pathway compounds that increase gene expression of TRPA1, or TRPM8, decrease gene expression of TRPV1, or any combination thereof, include curcumin, B-caryophyllene, N-palmitoylethanolamide, N-oleoylethanolamide, N-alkylamides, honokiol, docosahexaenoic acid (DHA), eicosapentaenoic acid (EPA), triptolide, diosphenol, isomenthone, menthone, limonene, myristicin, ginsenoside, ginkolide, eugenol, menthol, camphor, methyl salicylate, and any combination thereof.

In another embodiment, ECS related TRP pathway compounds that increase gene expression of TRPA1 include curcumin, N-palmitoylethanolamide, N-oleoylethanolamide, N-alkylamides, honokiol, myristicin, docosahexaenoic acid (DHA), eicosapentaenoic acid (EPA), triptolide, ginsenoside, ginkolide, eugenol, menthol, camphor, methyl salicylate, and any combination thereof.

In another embodiment, ECS related TRP pathway compounds that increase gene expression of TRPM8 include curcumin, B-caryophyllene, N-palmitoylethanolamide, honokiol, docosahexaenoic acid (DHA), eicosapentaenoic acid (EPA), triptolide, ginkolide, diosphenol, isomenthone, menthone, limonene, eugenol, and any combination thereof.

In another embodiment, ECS related TRP pathway compounds that decrease gene expression of TRPV1 include B-caryophyllene, diosphenol, isomenthone, menthone, limonene, and any combination thereof.

In another embodiment, ECS related TRP pathway compounds that increase gene expression of TRPA1, or TRPM8, decrease gene expression of TRPV1, or any combination thereof, include curcumin, B-caryophyllene, diosphenol, isomenthone, menthone, limonene, N-palmitoylethanolamide, triptolide, N-alkylamides, 7-hydroxyflavone, N-oleoylethanolamine and docosahexaenoic acid (DHA), eicosapentaenoic acid (EPA), and any combination thereof.

Further, compounds affecting TRPV1, TRPA1 and TRPM8 gene expression can be classified by chemical class. In such embodiment, the chemical classes for modulating TRPV1, TRPA1 and TRPM8 gene expression include N-alkylamides and polyunsaturated fatty acids (PUFAs).

In one embodiment, the N-alkylamides chemical class includes, but is not limited to, dodeca-2E,4E,8Z,10Z-tetraenoic acid isobutylamide and/or dodeca-2E,4E-dienoic acid isobutylamide.

In another embodiment the PUFAs chemical class includes, but is not limited to, docosahexaenoic acid (DHA), eicosapentaenoic acid (EPA), alpha-linolenic acid (ALA), eicosatetraenoic acid (ETA), oleic acid, palmitoleic acid, and vaccenic acid.

In some embodiments, a composition including one or more endocannabinoid mimetic direct compounds, one or more endocannabinoid indirect compounds, one or more ECS related pathway anti-inflammatory compounds, and one or more ECS related TRP pathway compounds modulates CERS, CASP8, and/or MAPK/ERK pathways, which regulate, for example, cellular proliferation, differentiation, autophagy, apoptosis, senescence, lipid synthesis and (indirectly) skin microbiome, is provided. Such compositions can exhibit gene modulating effects for COL1A1: (+), ITGB1: (+), JUN (−), KLF4: (+), CERS3: (+), FLG: (+), TLR2 (+) (with beneficial gene modulation indicated in parentheses, “+” for increase and “−” for decrease).

In one embodiment, a composition including at least one direct endocannabinoid compound, at least one indirect endocannabinoid compound, at least one ECS related pathway anti-inflammatory compound, and at least one ECS related TRP pathway compound provide beneficial gene expression for genes affecting skin matrix function measured by at least one gene selected from the group consisting of COL1A1, ITGB1, JUN, KLF4, CERS3, FLG, and TLR2, is provided. The composition can contain curcumin, B-caryophyllene, N-palmitoylethanolamide, N-oleoylethanolamide, N-alkylamides, 7-hydroxyflavone, honokiol, magnolol, diosphenol, docosahexaenoic acid (DHA), eicosapentaenoic acid (EPA), triptolide, ginsenoside, epigallocatechin gallate, apigenin, pentacyclic triterpene alcohols and triterpendiol monoesters including faradiol esters, eugenol, and any combination thereof.

In another embodiment, compounds for increasing COL1A1 gene expression include honokiol and/or magnolol.

In another embodiment, compounds for increasing ITGB1 gene expression include honokiol, magnolol, triterpene alcohols and triterpendiol monoesters including faradiol, and any combination thereof.

In another embodiment, compounds for decreasing JUN gene expression include N-palmitoylethanolamide, triterpene alcohols and triterpendiol monoesters including faradiol, docosahexaenoic acid (DHA), eicosapentaenoic acid (EPA), triptolide, and any combination thereof.

In another embodiment, compounds for increasing KLF4 gene expression include N-palmitoylethanolamide, honokiol, magnolol, docosahexaenoic acid (DHA), eicosapentaenoic acid (EPA), triptolide, and any combination thereof.

In another embodiment, a composition comprising at least one curcuminoid, at least one fatty acid amide, at least one sesquiterpene, at least one flavan-3-ol, at least one diterpene, at least one hydroxyflavone and at least one PUFA demonstrates therapeutically beneficial skin matrix gene modulation for the genes COL1A1 (+), ITGB1 (+), JUN (−) and KLF4 (+) (with beneficial gene modulation indicated in parentheses, “+” for increase and “−” for decrease) is provided.

In one embodiment, a composition including at least one direct endocannabinoid compound, at least one indirect endocannabinoid compound, at least one ECS related pathway anti-inflammatory compound, and at least one ECS related TRP pathway compound provides beneficial gene expression for genes modulating skin barrier function, lipid synthesis and antimicrobial properties measured by at least one gene selected from the group consisting of TLR2, TLR3, TGB1, CERS3, FLG, NRF2, PLA-2, KLK-7, PAR-2, TGM-1, IVL, ZNF750, SMPD1, DGAT2, GPAT3, HAS3, CLDN1, CDHR1, and DSG1, is provided. The composition can contain curcumin, B-caryophyllene, N-palmitoylethanolamide, N-oleoylethanolamide, N-alkylamides, 7-hydroxyflavone, honokiol, magnolol, diosphenol, docosahexaenoic acid (DHA), eicosapentaenoic acid (EPA), triptolide, ginsenoside, epigallocatechin gallate, apigenin, pentacyclic triterpene alcohols and triterpendiol monoesters including faradiol esters, eugenol, and any combination thereof.

In another embodiment, compounds for increasing CERS3 gene expression include N-palmitoylethanolamide, honokiol, magnolol, triterpene alcohols and triterpendiol monoesters including faradiol, docosahexaenoic acid (DHA), eicosapentaenoic acid (EPA), and any combination thereof.

In another embodiment, compounds for increasing FLG gene expression include N-palmitoylethanolamide, honokiol, magnolol, triterpene alcohols and triterpendiol monoesters including faradiol, docosahexaenoic acid (DHA), eicosapentaenoic acid (EPA), triptolide, and any combination thereof.

In another embodiment, compounds for increasing TLR2 gene expression include honokiol, magnolol, docosahexaenoic acid (DHA), eicosapentaenoic acid (EPA), and any combination thereof.

In another embodiment, an inventive composition comprising at least one curcuminoid, at least one fatty acid amide, at least one sesquiterpene, at least one flavan-3-ol, at least one diterpene, at least one hydroxyflavone, and at least one PUFA, is provided, which when delivered in an effective amount demonstrates beneficial skin barrier gene modulation for the genes CERS3 (+), FLG (+), and TLR2 (+).

In aspects, compositions of the invention modulate senescence-associated secretory phenotype (SASP) or elements thereof, such as proinflammatory cytokines, chemokines, growth factors, and proteases, protein p16, p53, TP53, MDM2, Matrix metalloproteinase MMP12, Senescence-associated beta-galactosidase (SA-β-Gal), or p21 levels. In aspects, compositions of the invention promote or cause selective elimination of senescent cells (senolytics) or the disruptions of the SASP. In aspects, compositions of the invention modulate autophagy. In aspects, compositions of the invention modulate prejuvenation, e.g., by helping prolong the optimal function or delay the decline of optimal function of cells.

In one embodiment, the composition includes at least one direct endocannabinoid compound, at least one indirect endocannabinoid compound, at least one ECS related pathway anti-inflammatory compound, and at least one compound that provides cellular senescence benefits measured by at least one gene selected from the group consisting of GLB1 (SA-β-Gal), CDKN2A (p16), CDKN1A (p21), TP53(p53), and MDM2 (modulates TP53). Preferred compounds include curcumin, B-caryophyllene, N-palmitoylethanolamide, N-oleoylethanolamide, N-alkylamides, 7-hydroxyflavone, 3,7 dihydroxyflavone, honokiol, magnolol, diosphenol, isomenthone, menthone, limonene, triptolide, ginsenoside, epigallocatechin gallate, apigenin, pentacyclic triterpene alcohols and triterpendiol monoesters including faradiol esters, eugenol, and any combination thereof.

In some embodiments, the composition of the invention includes direct endocannabinoid compounds (agonistic or antagonistic modulation of CB1 or CB2 gene expression), indirect endocannabinoid compounds (compounds that modulate metabolism of the primary endogenous ECS ligands, AEA or 2AG), ECS related pathway compounds (compounds affecting anti-inflammatory nuclear, enzymatic, and/or cytokine pathways), and ECS related TRP pathway compounds affecting skin barrier function, pain and cellular proliferation, differentiation, autophagy, apoptosis and/or senescence. (See, e.g., FIGS. 1 and 2 for a more complete list of ECS ligands, receptors, and enzymes.). Without wishing to be bound to theory, modulating multiple pathways with different compounds, and/or natural extracts containing such compounds, in a composition can increase the efficaciousness of the composition. Examples of ECS receptor activities exhibited by compositions of the invention include (1) modulation of expression of the CB1 and/or CB2 genes, (2) affecting the anandamide metabolic pathway by modulating expression of the NAPE-PLD or FAAH gene, and/or affecting the 2AG metabolic pathway by modulating expression of the MAGL or DAGL genes; (3) affecting an inflammation-related nuclear pathway by modulating expression of the PPARgamma gene, affecting an inflammation-related enzymatic pathway by modulating expression of at least one gene selected from the group consisting of COX1 (i.e., PTGS1), COX2, iNOS, 5-LOX, 12-LOX, NF-kappaB, a matrix metalloprotease (MMP), or affecting an inflammation-related cytokine pathway by modulating expression of at least one gene selected from the group consisting of IL-1beta, IL-1a, IL-6, IL-8, TNFa, IL-10 gene, and (4) affecting a TRP related pathway and thereby beneficially affecting skin cellular proliferation, differentiation, autophagy, apoptosis and/or senescence by modulating expression of at least one gene selected from the group consisting of TRPA, TRPM, and TRPV nocioceptors. Compositions can include compounds and/or natural extracts containing such compounds that affect two, three, four, or more of these pathways.

In some embodiments, the composition of the invention includes one or more direct endocannabinoid compounds (agonistic modulation of CB1 or CB2 gene expression), one or more indirect endocannabinoid compounds (compounds that modulate metabolism of the primary endogenous ECS ligands, AEA or 2AG, via antagonistic modulation of FAAH and MAGL), ECS related pathway compounds, (compounds modulating anti-inflammatory nuclear (agonistic modulation), enzymatic (antagonistic modulation), and/or cytokine (antagonistic modulation) pathways), and one or more ECS related TRP pathway compounds (compounds that decrease gene expression for TRPV1 (antagonistic), decrease gene expression for TRPV3 (antagonistic), increase gene expression for TRPV4 (agonistic), increase gene expression for TRPV6 (agonistic), increase gene expression for TRPA1 (agonistic), increase gene expression for TRPM8 nocioceptors (agonistic) or any combination thereof (See FIGS. 1 and 2 for a more complete list of ECS ligands, receptors, and enzymes). In aspects, a composition includes compounds and/or natural extracts containing such compounds that affect two, three, four, five, six, or more of these pathways. Modulating multiple pathways with different compounds and/or natural extracts containing such compounds can increase (e.g., synergistically) the efficaciousness of the composition as measured by gene modulation. Examples of ECS receptor modulation include (1) increasing expression of the CB1 and/or CB2 genes, (2) affecting the anandamide metabolic pathway by decreasing expression of the FAAH gene, and/or affecting the 2AG metabolic pathway by decreasing expression of the MAGL gene; (3) affecting an inflammation-related nuclear pathway by increasing expression of at least one gene selected from the group consisting of PPARg, PPARa, and PPARb, affecting an inflammation-related enzymatic pathway by decreasing expression of at least one gene selected from the group consisting of COX1 (i.e., PTGS1), COX2, iNOS, 5-LOX, 12-LOX, NF-kappaB, a matrix metalloprotease (MMP), or affecting an inflammation-related cytokine pathway by decreasing expression of at least one gene selected from the group consisting of IL-1beta, IL-1a, IL-6, IL-8, TNFa, or increasing expression of the IL-10 gene, (4) affecting a TRP pain or cellular proliferation, differentiation, autophagy, apoptosis, and senescence related pathway by selecting at least one compound that decreases gene expression from the group consisting of TRPV1 and/or TRPV3, and/or at least one compound that increases gene expression from the group consisting of TRPV4, TRPV6, TRPA1, and/or TRPM8 nocioceptors.

In some embodiments, the composition can be tailored to upregulate or downregulate expression of one or more genes of interest. For example, upregulation of CB1 can accelerate the repair of the skin's barrier but it can also increase fibrosis and scar formation. So while upregulation to speed wound healing is generally desirable, in people who are predisposed to develop scars then antagonists of CB1 can be useful to slow wound healing in order to reduce the risk or normalize wound healing to avoid the development of scars. Upregulation of CB2 stimulates lipid production which can be useful for therapeutics to treat dry skin whereas antagonists reduce oil production and may help in the treatment and reduction of acne. Accordingly, in some embodiments, the composition increases expression of one or more genes of interest, such as CB1 and/or CB2, and decreases expression of those genes in other embodiments.

Moreover, cytokines and inflammatory enzymes such as COX and MMP1 are known modulators of inflammatory stress, and down regulation leads to decreased inflammation, i.e. an “anti-inflammatory” response. However, in some situations, such as the very initial stages of wound healing, a pro-inflammatory response is desirable (i.e. an increase in cytokines) and is an important part of the necessary wound healing progression of events. In one aspect, compositions of the invention that upregulate MMPP1 include those that contain a curcuminoid, a fatty acid amide, a biphenol, a sesquiterpene, a diterpene, and a PUFA. In another aspect, the composition contains a curcuminoid, a fatty acid amide, a biphenol, a sesquiterpene, a flavan-3-ol, a diterpene, and a PUFA. In another aspect, the composition contains a curcuminoid, a fatty acid amide, a biphenol, a sesquiterpene, a flavan-3-ol, a hydroxyflavone, a diterpene, and a PUFA. In another aspect, the composition contains a curcuminoid, a fatty acid amide, a sesquiterpene, a diterpene, and a PUFA. Upregulation of PPARg has been shown to promote binding of retinoid receptors RXR and to regulate important cellular functions including cell proliferation, and differentiation, and inflammatory responses. PPARg plays an important role in regulating homeostasis and modulation of PPARg can be beneficial through upregulation by suppressing proliferation and inducing differentiation of keratinocytes in psoriasis patients. In contrast downregulating PPARg expression by fibroblasts enhances dermal wound closure. Compositions of the invention that downregulate PPARg include those that contain a curcuminoid, a fatty acid amide, a sesquiterpene, a diterpene, and a PUFA (or) those that contain curcuminoid, a fatty acid amide, a sesquiterpene, a diterpene, a hydroxyflavone and a PUFA. Accordingly, in some embodiments, the composition increases expression of one or more genes of interest, such as cytokines and inflammatory enzymes, and decreases expression of those genes in others.

In some embodiments, combinations of compounds are selected/designed to maximize the number of ECS pathways [See, e.g., FIG. 2] for a specific desired therapeutic outcome, such pathways include, but are not limited to, compounds that modulate CB1 & CB2 gene expression, compounds that modulate ECS indirect ligand (AEA & 2AG) metabolism, compounds that modulate ECS related pathway inflammatory response, compounds that modulate ECS related TRP pathways including pain, itch, cellular proliferation, differentiation, autophagy, apoptosis, and senescence, wound healing, barrier function, and skin microbiome. Such cellular modulation of cellular proliferation, differentiation, autophagy, apoptosis, and senescence is particularly important in keratinocytes.

In certain embodiments, gene expression results are surprisingly improved in response to compound combinations in comparison to individual compound test data, thereby indicating that unique combinations of compounds within a composition can modulate gene expression in a novel manner; in many cases, such gene modulation results are synergistically greater than the sum of the individual compounds tested. In certain embodiments, for inventive compositions containing combinations of four compounds and/or natural extracts containing such compounds, one direct receptor endocannabinoid mimetic compound, a second indirect endocannabinoid mimetic compound that affects an ECS endogenous ligand metabolic pathway, a third endocannabinoid mimetic compound that affects an ECS related pathway anti-inflammatory compound, and fourth endocannabinoid mimetic compound that affects an ECS related TRP pathway (e.g., a pain, itch, cellular proliferation, differentiation, autophagy, apoptosis, senescence, skin matrix, lipid synthesis or barrier repair pathway) can be selected/incorporated. In one embodiment of a composition containing five compounds, one selected from a direct receptor endocannabinoid mimetic compound, a second selected from an indirect endocannabinoid mimetic compound that affects an ECS endogenous ligand metabolic pathway, a third selected from an endocannabinoid mimetic compound that affects an ECS related pathway anti-inflammatory compound can be selected, a fourth from an endocannabinoid mimetic compound that affects an ECS related TRP pathway, and a fifth selected compound is another direct endocannabinoid mimetic compound, an indirect endocannabinoid mimetic compound that affects an endogenous ligand metabolic pathway, an endocannabinoid mimetic compound that affects an ECS related anti-inflammatory compound, a endocannabinoid mimetic compound that affects an ECS related TRP pathway, or a endocannabinoid mimetic compound for a pathway not affected by the first four compound selections.

Regarding compositions containing combinations of five or more compounds, the same approach can be used to maximize the number of affected pathways (e.g., endocannabinoid system direct, indirect, anti-inflammatory, and ECS related TRP pathways) to produce a composition effective for wound healing. In one embodiment, such a combination includes curcumin, B-caryophyllene, N-palmitoylethanolamide, honokiol, magnolol, epigallocatechin gallate, apigenin, docosahexaenoic acid (DHA), eicosapentaenoic acid (EPA), and triptolide, which produced beneficial gene results for the skin matrix and barrier gene set. Summarily the test results were: Superior gene expression response compared to cannabidiol CBD for CERS3, COL1A1, FLG, ILIA, ITGB1, JUN, & KLF4; synergistically superior to individual composition compounds in gene response for COL1A1, ITGB1, & KLF4; a strong increase in MMP associated consistent with initial wound healing expectations; and more effective agonist for COL1A1 and KLF4 than cannabidiol.

Regarding compositions containing a combination of five or more compounds and/or natural extracts (e.g., six or more, seven or more, eight or more, nine or more, or ten or more), the same approach can be followed, with consideration to, for instance, maximize a specific skin condition benefit. Compounds can be selected from direct endocannabinoid mimetic compounds, indirect endocannabinoid mimetic compounds, ECS related anti-inflammatory endocannabinoid mimetic compounds, ECS related TRP pathway compounds, endocannabinoid mimetic compounds modulating cellular proliferation, differentiation, autophagy, apoptosis, senescence, lipid synthesis or barrier repair pathways, and endocannabinoid mimetic compounds modulating skin matrix pathways are utilized to develop such compositions. In one embodiment, the composition includes combining curcumin, B-caryophyllene, diosphenol, isomenthone, menthone, limonene, N-palmitoylethanolamide, triptolide, docosahexaenoic acid (DHA), and eicosapentaenoic acid (EPA). This composition synergistically improved the increased gene expression for CB1 and synergistically decreased gene expression for MAGL and demonstrated highly effective positive TRPM8 nocioreceptor gene expression; better than those observed against equivalent concentrations of cannabidiol. In another embodiment, the composition includes curcumin, B-caryophyllene, N-palmitoylethanolamide, triptolide, 7-hydroxyflavone, N-oleoylethanolamine, docosahexaenoic acid (DHA), and eicosapentaenoic acid (EPA), which produced beneficial gene expression for direct endocannabinoid receptors (CB1, CB2) and indirect endocannabinoid receptors (FAAH, MAGL), and in all cases more effective than equivalent concentrations of cannabidiol, and additionally produced synergistically beneficial results for the cytokine anti-inflammatory marker NFKB, the enzymatic anti-inflammatory marker COX1 (PTGS1), and downregulation of TRPV1, in all cases superior to equivalent concentrations of cannabidiol. Both examples are suitable for use as an anti-inflammatory, pain & itch relief, and restoration of ECS homeostasis for cellular proliferation, differentiation, autophagy, apoptosis, senescence, lipid synthesis and barrier function.

In one embodiment, the composition includes a combination of compounds and/or natural extracts are selected from the groups consisting of direct and indirect endocannabinoid compounds, anti-inflammatory compounds from each of the three ECS related anti-inflammatory pathways (nuclear, enzymatic, and cytokine) and the ECS related TRP pathway compounds for pain, itch, cellular proliferation, differentiation, autophagy, apoptosis, senescence, lipid synthesis and barrier function compounds. Particularly, the composition includes B-caryophyllene, curcumin, N-palmitoylethanolamide, diosphenol, isomenthone, menthone, limonene, docosahexaenoic acid (DHA), eicosapentaenoic acid (EPA), and triptolide, which produced surprisingly synergistic beneficial gene test results for CB1 and MAGL expression (see test Example 1, composition 2). The group designation for the composition is:
Direct ECS pathway (CB1, CB2): Curcuminoids: curcumin
Indirect ECS pathway (FAAH, MAGL): Fatty Acid Amides: N-palmitoylethanolamide
ECS related Anti-inflammatory nuclear pathway (PPARg): Monoterpenes: diosphenol, isomenthone, menthone, limonene
ECS related Anti-inflammatory enzymatic pathway (PTGS1): Sesquiterpenes: B-caryophyllene
ECS related Anti-inflammatory cytokine pathway: (ILIA, NFKB): Diterpenes: Triptolide
ECS related TRP pathway: (TRPV1, TRPA1, TRPM8): PUFAs: docosahexaenoic acid (DHA), eicosapentaenoic acid (EPA)

In another embodiment, the composition includes a combination of compounds are selected from the groups consisting of direct and indirect endocannabinoid compounds, anti-inflammatory compounds from each of the three ECS related anti-inflammatory pathways (nuclear, enzymatic, and cytokine) and the ECS related TRP pathway compounds for pain, itch, cellular proliferation, differentiation, autophagy, apoptosis, senescence, lipid synthesis and barrier function compounds. Particularly, the composition includes B-caryophyllene, curcumin, N-palmitoylethanolamide, N-oleoylethanolamide, 7-hydroxyflavone, docosahexaenoic acid (DHA), eicosapentaenoic acid (EPA), and triptolide produced synergistic beneficial gene test results for NFKB, PTGS1 and TRPV1 expression (see test Example 1, composition 3). The group designation for the composition is:
Direct ECS pathway (CB1, CB2): Curcuminoids: curcumin
Indirect ECS pathway (FAAH, MAGL): Fatty Acid Amides: N-oleoylethanolamide, N-palmitoylethanolamide
ECS related Anti-inflammatory nuclear pathway (PPARg): Monoterpenes: diosphenol, isomenthone, menthone, limonene
ECS related Anti-inflammatory enzymatic pathway (PTGS1): Sesquiterpenes: B-caryophyllene
ECS related Anti-inflammatory cytokine pathway: (ILIA, NFKB): Diterpenes: Triptolide; Hydroxyflavones: 7-hydroxyfalvone
ECS related TRP pathway: (TRPV1, TRPA1, TRPM8): PUFAs: docosahexaenoic acid (DHA), eicosapentaenoic acid (EPA)

Some compositions of the invention, comprised of ECS direct, indirect, related inflammatory pathway and TRP related pathway compounds, modulate beneficial gene expression in fibroblasts across multiple genes (e.g. CB1, CB2, FAAH, MAGL, IL1a, NFKB, PRGS1, TRPM8, & TRPV1) significantly better (e.g. 54 fold better for CNR1, 33 fold better for CNR2, 15 fold better for MAGL, 10 fold better for TRPM8) than cannabidiol demonstrating that phyto-mimetic non-cannabinoid compounds of the invention are beneficially modulating the ECS more effectively than traditional cannabinoids. See Test examples 1, compositions 2 and 3.

In another embodiment, the composition includes a combination of compounds designed for anti-inflammatory, anti-aging, skin matrix improvement, and wound healing selected from the groups consisting of direct and indirect endocannabinoid compounds, anti-inflammatory compounds from each of the three ECS related anti-inflammatory pathways (nuclear, enzymatic, and cytokine), and the ECS related TRP pathways. Particularly, the composition includes curcumin, B-caryophyllene, N-palmitoylethanolamide, honokiol, magnolol, epigallocatechin gallate, apigenin, docosahexaenoic acid (DHA), eicosapentaenoic acid (EPA), triptolide, which produced surprisingly synergistic beneficial gene test results for COL1A, ITGB1 and KLF4 (see test Example 3, composition 8). The group designation for the composition is:
Direct ECS pathway (CB1/CB2): Curcuminoids: curcumin
Indirect ECS pathway (FAAH, MAGL): Fatty Acid Amides: N-palmitoylethanolamide
ECS related Anti-inflammatory nuclear pathway (PPARg): Biphenols: honokiol, magnolol
ECS related Anti-inflammatory enzymatic pathway (PTGS1): Sesquiterpenes: B-caryophyllene; Flavan-3-ols: EGCG
ECS related Anti-inflammatory cytokine pathway: (ILIA, NFKB): Diterpenes: Triptolide Hydroxyflavones: Apigenin
ECS related TRP pathway: (TRPV1, TRPA1, TRPM8): PUFAs: docosahexaenoic acid (DHA), eicosapentaenoic acid (EPA)

Additionally, these compositions, each of which included at least one curcuminoid, at least one fatty acid amide, at least one biphenol, at least one sesquiterpene, at least one flavan-3-ol, at least one diterpene, at least one hydroxyflavone and at least one PUFA demonstrated beneficial skin matrix gene modulation for the genes COL1A1 (+), ITGB1 (+), JUN (−) and KLF4 (+) and beneficial skin barrier gene modulation for the genes CERS3 (+), FLG (+), and TLR2 (+).

Similarly, compositions of the invention, modulate beneficial gene expression in keratinocytes across multiple genes regulating skin matrix function, including those for lipid regulation/barrier repair (CERS3, FLG, TLR2), inflammatory response (IL1a, NFKB, MMP1) and structural pathway genes affecting extracellular matrix protein metabolism and cellular proliferation, differentiation and apoptosis (COL1A1, ITGB1, JUN, KLF4) superior to results for cannabidiol in CERS3, COL1A1, FLG, ILIA, ITGB1, JUN, & KLF4, again demonstrating that phyto-mimetic non-cannabinoid compounds of the invention are beneficially modulating the ECS more effectively than traditional cannabinoids. See Test example 3, composition 8.

In one embodiment the composition contains at least one direct endocannabinoid mimetic compound, wherein the at least one compound is demethoxycurcumin, bisdemethoxycurcumin, tetrahydrocurcumin, eugenol, or any combination thereof, and wherein the at least one compound is optionally contained in one or more of the following extracts: Curcuma longa, Curcuma xanthorrhiza, Curcuma zedoaria, Eugenia caryophyllata, Syzygium aromaticum, Myristica fragrans, Cinnamomum verum, Ocimum basilicum, and Laurus nobilis; at least one indirect endocannabinoid mimetic compounds, wherein the at least one compound is N-oleoylethanolamide (OEA), oleamide, arachidonamide, or any combination thereof, and wherein the at least one compound is optionally contained in one or both of the following extracts: Theobroma cacao and Achyranthes aspera; at least one ECS related pathway anti-inflammatory compound, wherein the at least one compound is diosphenol, limonene, isomenthone, menthone, resveratrol, humulene (alpha-caryophyllene), ginkolide, bilobalide, helenalin, parthenolide, triptolide, carnosic acid, or any combination thereof, and wherein the at least one compound is optionally contained in one or more of the following extracts: Agathosma betulina, Agathosma crenulata, Vitis Vinifera L., Vaccinium sp., Humulus lupulus, Ginkgo biloba, Arnica montana, Tanacetum parthenium, Tripterygium wilfordii, Salvia mellifera, Rosmarinus officinalis, and Salvia officinalis; and an ECS related TRP pathway compound that is oleic acid, palmitoleic acid, vaccenic acid, or any combination thereof, and wherein the at least one compound is optionally contained in one or both of the following extracts: Olea europaea and Macadamia integrifolia.

In one embodiment the composition contains a direct endocannabinoid mimetic compound that is eugenol, and wherein the at least one compound is optionally contained in one or more of the following extracts: Eugenia caryophyllata, Syzygium aromaticum, Myristica fragrans, Cinnamomum verum, Ocimum basilicum, and Laurus nobilis; at least one indirect endocannabinoid mimetic compound, wherein the at least one compound is oleamide, arachidonamide, or both; at least one ECS related pathway anti-inflammatory compound, wherein the at least one compound is diosphenol, resveratrol, ginkolide B, bilobalide, helenalin, parthenolide, triptolide carnosic acid, or any combination thereof, and wherein the at least one compound is optionally contained in one or more of the following extracts: Agathosma betulina, Agathosma crenulata, Vitis Vinifera L., Vaccinium sp., Ginkgo biloba, Arnica montana, Tanacetum parthenium, Tripterygium wilfordii, Salvia mellifera, Rosmarinus officinalis, and Salvia officinalis; and at least one ECS related TRP pathway compound, wherein the at least one compound is oleic acid, palmitoleic acid, vaccenic acid, or any combination thereof, and wherein the at least one compound is optionally contained in one or both of the following extracts: Olea europaea, and Macadamia integrifolia.

In one embodiment the composition contains at least one direct endocannabinoid mimetic compound, wherein the at least one compound is tetrahhydrocurcumin or eugenol, and wherein the at least one compound is optionally contained in one or more of the following extracts: Eugenia caryophyllata, Syzygium aromaticum, Myristica fragrans, Cinnamomum verum, Ocimum basilicum, or Laurus nobilis; an indirect endocannabinoid mimetic compound that is oleoylethanolamide, and wherein the compound is optionally contained in one or both of the following extracts: Theobroma cacao and Achyranthes aspera; at least one ECS related pathway anti-inflammatory compound, wherein the at least one compound is diosphenol, ginkolide B, triptolide, or any combination thereof, and wherein the at least one compound is optionally contained in one or more of the following extracts: Agathosma betulina, Agathosma crenulata, Ginkgo biloba and Tripterygium wilfordii; and at least one ECS related TRP pathway compound, wherein the at least one compound is oleic acid, palmitoleic acid, vaccenic acid, or any combination thereof, and wherein the at least one compound is optionally contained in one or more of the following extracts: Olea europaea, and Macadamia integrifolia.

In one embodiment the composition contains at least one direct endocannabinoid mimetic compound, wherein the at least one compound is an allyl chain substituted guaiacol (preferably eugenol or at least one eugenol-containing natural extract such as Eugenia caryophyllata, Syzygium aromaticum, Myristica fragrans, Cinnamomum verum, Ocimum basilicum, Laurus nobilis, or any combination thereof); at least one indirect endocannabinoid mimetic compound, wherein the at least one compound is a diterpene (preferably triptolide or the triptolide-containing natural extract Tripterygium wilfordii); at least one ECS related pathway anti-inflammatory compound, wherein the at least one compound is a monoterpene (preferably diosphenol or at least one diosphenol-containing natural extract such as Agathosma betulina or Agathosma crenulata), an N-alkylamide (preferably isobutylamide or at least one isobutylamide-containing natural extract such as Echinacea purpurea), a hydroxyflavone (preferably 7-hydroxyflavone or 3,7-dihydroxyflavone or at least one 7-hydroxyflavone or 3,7-dihydroxyflavone-containing natural extract such as Daemonorops draco, Dracaena cochinchinensis, or both), or any combination thereof; and at least one ECS related TRP pathway compound, wherein the at least one compound is a fatty acid amide (preferably oleoylethanolamide or an oleoylethanolamide-containing natural extract such as Theobroma cacao, Achyranthes aspera, or both).

Compositions of the invention modulate gene expression for ECS direct, ECS indirect, ECS related inflammatory and ECS related TRP pathways. In an aspect, a composition according to the invention produces a fold change of at least about 2, 3, 5, 10, 15, 20, 30, 40, 50, 60, 70, or 80 in the expression level of the ECS direct pathway genes CB1 and/or CB2. In an aspect, a composition according to the invention produces a fold change of at least, e.g., about 2, 3, 4, 5, 10, 15, or 20 in the expression level of the ECS indirect pathway genes FAAH and/or MAGL. In an aspect, a composition according to the invention produces a fold change of at least about 2, 3, 4, 5, 6, 7, 8, 9 or 10 in the expression level of one or more of the ECS related inflammatory pathway genes IL1a, NFKB and PTGS1. In an aspect, a composition according to the invention produces a fold change of at least about 2, 3, 5, 10, 15, 20, 25, 30, 40, 50, 60, 70, 80, 90 or 100 in the expression level of the ECS related TRP pathway genes TRPV1, TRPA1 or TRPM8. See, e.g., Test example 1, compositions 2 and 3. Fold changes can be determined by comparing a gene of interest's expression level in a cell treated with the composition compared to the gene of interest's expression level in the same type of cell not treated with the composition.

In one aspect, compositions of the invention that produce one or more of the herein-mentioned fold changes in gene expression include those that contain at least one direct ECS compound that is a curcuminoid, an allyl chain substituted guaiacol, or any combination thereof; at least one indirect ECS compound that is a fatty acid amide, a ginsenoside, or any combination thereof; at least one ECS related anti-inflammatory compound that is a monoterpene, a biphenol, a sesquiterpene, a terpene lactone, a flavan-3-ol, a hydroxyflavone, a diterpene, a triterpene, or any combination thereof; and at least one ECS related TRP pathway compound that is an N-alkylamide, a PUFA, or any combination thereof. In another aspect, compositions of the invention that produce fold changes in the gene expression of CB1, CB2, FAAH, MAGL, or any combination thereof, contain a curcuminoid, a fatty acid amide, a sesquiterpene, a monoterpene, a diterpene, and a PUFA. In another aspect, compositions of the invention that produce fold changes in the gene expression of IL1a, NFKB PTGS1, TRPV1, TRPA1, TRPM8, or any combination thereof, contain a curcuminoid, a fatty acid amide, a sesquiterpene, a diterpene, and a PUFA, or, alternatively, the composition can contain a curcuminoid, a fatty acid amide, a sesquiterpene, a hydroxyflavone, a diterpene, and a PUFA.

In certain embodiments, when the cosmetic or pharmaceutical composition comprising a compound and/or natural extract blend comprises at least a first compound or natural extract having a direct endocannabinoid mimetic receptor effect (preferably two or more or three or more), a second endocannabinoid mimetic compound or natural extract having an indirect endogenous ligand metabolic pathway effect (preferably two or more or three or more), a third endocannabinoid mimetic compound or natural extract with an ECS related pathway anti-inflammatory effect (preferably two or more or three or more), and a fourth endocannabinoid mimetic compound or natural extract having an ECS related TRP pathway effect, each of the endocannabinoid mimetic compounds and/or natural extracts are selected from groups described herein. In certain embodiments, the cosmetic or pharmaceutical composition comprises four compounds and/or natural extracts containing such compounds. Optionally, the cosmetic or therapeutic drug dermatological composition of this embodiment comprises four or more (e.g., five or more, or six or more, or seven or more, eight or more, nine or more, or ten or more) compounds and/or natural extracts containing such compounds, wherein each of the compounds and/or natural extracts are selected from those described herein. For instance, compositions with four compounds and/or natural extracts can include no compounds and four natural extracts, one compound and three natural extracts, two compounds and two natural extracts, three compounds and one natural extract, and four compounds and no natural extracts.

Certain embodiments of the inventive endocannabinoid mimetic composition provide a synergistic improvement (e.g., in comparison to the expected summation of effects of the compounds and/or natural extracts assessed individually as measured by target gene expression) in the capacity for an improvement in an ECS pathway (function) and/or an improvement in a ECS affected cell type; such improvements include oxidative stress and inflammation protection, and, in at least some embodiments, pain relief as well. Certain embodiments of the invention provide a superior improvement in specific gene expression to provide a beneficial effect on the human endocannabinoid system via direct endocannabinoid receptor activation, indirect modulation of endogenous ECS ligands and/or positive influence on an ECS related pathways including providing a superior reduction in inflammation and pain relief compared to cannabidiol. In addition, certain embodiments of the invention provide for an improved skin regimen by providing broad spectrum ECS pathway and/or ECS affected cell types improvements including oxidative stress protection and/or anti-inflammatory benefits associated with endocannabinoid mimetic compounds and/or extracts in a single composition. Certain functional use aspects of compositions of the invention, include, but are not limited to, anti-inflammatory benefits, pain and itch relief, improvement in skin matrix, barrier function, lipid synthesis, cellular proliferation, differentiation, autophagy, apoptosis, senescence, wound healing, restoration of ECS homeostasis. Benefits can be increased by the inclusion of additional compounds and/or natural extracts in the compositions, which can be specifically selected based on gene modulation test results for the appropriate indicated genes, e.g. compounds and/or natural extracts for the antagonistic effect on TRPV1 may reduce inflammation, pain and itch while improving barrier repair and increasing apoptosis.

In some embodiments, the composition includes one or more natural extracts known to contain one or more chemical compounds discussed herein. Compositions of the invention can be composed of one or more isolated compounds, one or more isolated compounds combined with one or more natural extracts containing compounds referred to herein, or one or more natural extracts containing compounds referred to herein. For instance, B-caryophyllene, an active compound, can be derived from numerous plant species including, by common name, clove, oregano, black pepper, basil, eugenol, patchouli and marijuana [see below for a more complete list]. natural extracts serve as an alternative natural source for the compound, or a source for multiple compounds in a single natural extract. In some embodiments, there may be multiple natural extracts that provide a compound. natural extracts selected for inclusion in a composition are based on known chemical content and thus have similar gene function activity by group as per their isolated compound counterparts, adjusted for concentration.

A list of exemplary compounds and suitable source natural extracts is indicated here:

B-Caryophyllene: Bidens pilosa, Syzygium aromaticum (Eugenia caryophyllata), Piper nigrum, Perilla frutescens, Rosmarinus officinalis, Lindera benzoin, Centella asiatica, Angelica archangelica, Coleus barbatus, Origanum vulgare, Ptychopetalum olacoides, Ocimum basilicum, Salvia officinalis, Vitex agnus-castus, Petroselinum crispum, Coriandrum sativum, Boswellia sacra, Apium graveolens, Eucalyptus citriodora, Piper cubeba, Cinnamomum verum, Thymus vulgaris, Myrrhis odorata, Pinus sylvestris, Valeriana officinalis, Aesculus hippocastanum, Murraya koenigii, Tagetes minuta, Tamarindus indica, Melaleuca alternifolia, Mentha longifolia, Citrus limon, Ocimum tenuiflorum, Tagetes filifolia, Hedychium flavum, Eucalyptus tetraptera, Micromeria fruticosa, Salvia triloba, Artemisia annua, Salvia canariensis, Pogostemon cablin, and Copaifera officinalis.
curcumin: Curcuma longa, Curcuma xanthorrhiza, Curcuma zedoaria
N-palmitoylethanolamide: Glycine max, Arachis hypogaea, Gallus gallus domesticus (egg oil)
docosahexaenoic acid (DHA), dicosapentaenoic acid, (EPA): Schizochytrium sp. including S. aggregatum, Clupea pallasii (Pacific Herring oil), Oncorhynchus tshawytscha (Chinook Salmon oil), Euphausia sp. (krill oil)
diosphenol: Agathosma betulina (preferred), Agathosma crenulata
7-Hydorxyflavone: Daemonorops draco (preferred), Dracaena cochinchinensis
N-oleoylethanolamine: Theobroma cacao, Achyranthes aspera
triptolide: Tripterygium wilfordii
honokiol/magnolol: Magnolia officinalis, Magnolia grandiflora, Magnolia dealbata, Magnolia biondii, Magnolia obovata
apigenin: Matricaria chamomilla, Petroselinum crispum, Allium cepa, Citrus Sinensis, Triticum aestivum
epigallocatechin gallate/EGCG: Camellia sinensis, Helianthemum glomeratum, Vaccinium oxycoccos, Fragaria ananassa, Rubus fruticosus, Actinidia deliciosa, Prunus avium, Pyrus sp., Prunus persica, Malus domestica, Persea americana, Carya illinoinensis, Pistacia vera, and Corylus avellana.

The endocannabinoid mimetic compounds of the present invention include compounds that directly affect the functioning of the endocannabinoid system by, e.g., increasing the expression of the Cannabinoid Receptor Type 1, CNR1 (CB1) gene or the Cannabinoid Receptor Type 2, CNR2 (CB2) gene. See FIG. 2 depicting a non-limiting schematic of the endocannabinoid system.

In one embodiment, the endocannabinoid mimetic natural extracts that increase CB1 or CB2 gene expression include extracts from Achyranthes aspera, Aesculus hippocastanum, Agathosma betulina, Agathosma crenulata, Angelica archangelica, Annona cherimola, Apium graveolens, Arachis hypogaea, Artemisia annua, Bidens pilosa, Boswellia sacra, Brassica sp., Calendula officinalism, Centella asiatica, Cinnamomum verum, Citrus limon, Clupea pallasii (Pacific Herring oil), Coleus barbatus, Copaifera officinalis, Coriandrum sativum, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Eugenia caryophyllata, Euphausia sp. (krill oil), Gallus gallus domesticus (egg oil), Ginkgo biloba, Glycine max, Hedychium flavum, Lindera benzoin, Magnolia biondii, Magnolia dealbata, Magnolia grandiflora, Magnolia obovate, Magnolia officinalis, Melaleuca alternifolia, Mentha longifolia, Micromeria fruticosa, Murraya koenigii, Myrrhis odorata, Ocimum basilicum, Ocimum tenuiflorum, Oncorhynchus tshawytscha (Chinook Salmon oil), Origanum vulgare, Perilla frutescens, Petroselinum crispum, Pinus sylvestris, Piper cubeba, Piper nigrum, Pogostemon cablin Ptychopetalum olacoides, Rosmarinus officinalis, Ruta graveolens, S. aggregatum, Salvia canariensis, Salvia divinorum, Salvia officinalis, Salvia triloba, Schizochytrium sp., Syzygium aromaticum (Eugenia caryophyllata), Tagetes filifolia, Tagetes minuta, Tamarindus indica, Theobroma cacao, Thymus vulgaris, Tripterygium wilfordii, Uncaria tomentosa, Valeriana officinalis, Vitex agnus-castus, and any combination thereof.

In another embodiment, natural extracts for increasing CB1 or CB2 gene expression include extracts of Aesculus hippocastanum, Angelica archangelica, Annona cherimola, Apium graveolens, Arachis hypogaea, Artemisia annua, Bidens pilosa, Boswellia sacra, Brassica sp., Calendula officinalis, Centella asiatica, Cinnamomum verum, Citrus limon, Coleus barbatus, Copaifera officinalis, Coriandrum sativum, Curcuma longa, Curcuma xanthorrhiza, Curcuma zedoaria, Echinacea purpurea, Eucalyptus citriodora, Eucalyptus tetraptera, Eugenia caryophyllata, Gallus gallus domesticus (egg oil), Ginkgo biloba, Glycine max, Hedychium flavum, Lindera benzoin, Magnolia biondii, Magnolia dealbata, Magnolia grandiflora, Magnolia obovata, Magnolia officinalis, Melaleuca alternifolia, Mentha longifolia, Micromeria fruticosa, Murraya koenigii, Myrrhis odorata, Ocimum basilicum, Ocimum tenuiflorum, Origanum vulgare, Perilla frutescens, Petroselinum crispum, Pinus sylvestris, Piper cubeba, Piper nigrum, Pogostemon cablin, Ptychopetalum olacoides, Rosmarinus officinalis, Ruta graveolens, Salvia canariensis, Salvia divinorum, Salvia officinalis, Salvia triloba, Syzygium aromaticum (Eugenia caryophyllata), Tagetes filifolia, Tagetes minuta, Tamarindus indica, Thymus vulgaris, Tripterygium wilfordii, Valeriana officinalis, Vitex agnus-castus, and any combination thereof.

In another embodiment, natural extracts for increasing CB1 or CB2 gene expression include extracts from Aesculus hippocastanum, Angelica archangelica, Apium graveolens, Arachis hypogaea, Artemisia annua, Bidens pilosa, Boswellia sacra, Centella asiatica, Cinnamomum verum, Citrus limon, Coleus barbatus, Copaifera officinalis, Coriandrum sativum, Curcuma longa, Curcuma xanthorrhiza, Curcuma zedoaria, Echinacea purpurea, Eucalyptus citriodora, Eucalyptus tetraptera, Eugenia caryophyllata, Gallus gallus domesticus (egg oil), Ginkgo biloba, Glycine max, Hedychium flavum, Lindera benzoin, Magnolia biondii, Magnolia dealbata, Magnolia grandiflora, Magnolia obovate, Magnolia officinalis, Melaleuca alternifolia, Mentha longifolia, Micromeria fruticosa, Murraya koenigii, Myrrhis odorata, Ocimum basilicum, Ocimum tenuiflorum, Origanum vulgare, Perilla frutescens, Petroselinum crispum, Pinus sylvestris, Piper cubeba, Piper nigrum, Pogostemon cablin, Ptychopetalum olacoides, Rosmarinus officinalis, Salvia canariensis, Salvia officinalis, Salvia triloba, Syzygium aromaticum (Eugenia caryophyllata), Tagetes filifolia, Tagetes minuta, Tamarindus indica, Thymus vulgaris, Tripterygium wilfordii, Valeriana officinalis, Vitex agnus-castus, and any combination thereof.

In another embodiment, natural extracts for increasing CB1 gene expression include extracts from Curcuma longa, Glycine max, Arachis hypogaea, Gallus gallus domesticus (egg oil), Magnolia officinalis, Ginkgo biloba, Eugenia caryophyllata, and any combination thereof.

In another embodiment, natural extracts for increasing CB2 gene expression include extracts from Aesculus hippocastanum, Angelica archangelica, Apium graveolens, Arachis hypogaea, Artemisia annua, Bidens pilosa, Boswellia sacra, Centella asiatica, Cinnamomum verum, Citrus limon, Coleus barbatus, Copaifera officinalis, Coriandrum sativum, Curcuma longa, Curcuma xanthorrhiza, Curcuma zedoaria, Echinacea purpurea, Eucalyptus citriodora, Eucalyptus tetraptera, Eugenia caryophyllata, Gallus gallus domesticus (egg oil), Glycine max, Hedychium flavum, Lindera benzoin, Magnolia biondii, Magnolia dealbata, Magnolia grandiflora, Magnolia obovate, Magnolia officinalis, Melaleuca alternifolia, Mentha longifolia, Micromeria fruticosa, Murraya koenigii, Myrrhis odorata, Ocimum basilicum, Ocimum tenuiflorum, Origanum vulgare, Perilla frutescens, Petroselinum crispum, Pinus sylvestris, Piper cubeba, Piper nigrum, Pogostemon cablin, Ptychopetalum olacoides, Rosmarinus officinalis, Salvia canariensis, Salvia officinalis, Salvia triloba, Syzygium aromaticum (Eugenia caryophyllata), Tagetes filifolia, Tagetes minuta, Tamarindus indica, Thymus vulgaris, Tripterygium wilfordii, Valeriana officinalis, Vitex agnus-castus, and any combination thereof.

In another embodiment, natural extracts for increasing CB1 gene expression include extracts from Achyranthes aspera, Aesculus hippocastanum, Agathosma betulina, Agathosma crenulata, Angelica archangelica, Apium graveolens, Arachis hypogaea, Artemisia annua, Bidens pilosa, Boswellia sacra, Centella asiatica, Cinnamomum verum, Citrus limon, Clupea pallasii (Pacific Herring oil), Coleus barbatus, Copaifera officinalis, Coriandrum sativum, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Eugenia caryophyllata Euphausia sp. (krill oil), Gallus gallus domesticus (egg oil), Ginkgo biloba, Glycine max, Hedychium flavum, Lindera benzoin, Magnolia biondii, Magnolia dealbata, Magnolia grandiflora, Magnolia obovate, Magnolia officinalis, Melaleuca alternifolia, Mentha longifolia, Micromeria fruticosa, Murraya koenigii, Myrrhis odorata, Ocimum basilicum, Ocimum tenuiflorum, Oncorhynchus tshawytscha (Chinook Salmon oil), Origanum vulgare, Perilla frutescens, Petroselinum crispum, Pinus sylvestris, Piper cubeba, Piper nigrum, Pogostemon cablin, Ptychopetalum olacoides, Rosmarinus officinalis, S. aggregatum, Salvia canariensis, Salvia officinalis, Salvia triloba, Schizochytrium sp., Syzygium aromaticum (Eugenia caryophyllata), Tagetes filifolia, Tagetes minuta, Tamarindus indica, Theobroma cacao, Thymus vulgaris, Tripterygium wilfordii, Valeriana officinalis, Vitex agnus-castus, and any combination thereof.

In another embodiment, natural extract for increasing CB2 gene expression include extracts from Achyranthes aspera, Aesculus hippocastanum, Agathosma betulina, Agathosma crenulata, Angelica archangelica, Apium graveolens, Arachis hypogaea, Artemisia annua, Bidens pilosa, Boswellia sacra, Centella asiatica, Cinnamomum verum, Citrus limon, Clupea pallasii (Pacific Herring oil), Coleus barbatus, Copaifera officinalis, Coriandrum sativum, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Eugenia caryophyllata, Euphausia sp. (krill oil), Gallus gallus domesticus (egg oil), Glycine max, Hedychium flavum, Lindera benzoin, Magnolia biondii, Magnolia dealbata, Magnolia grandiflora, Magnolia obovate, Magnolia officinalis, Melaleuca alternifolia, Mentha longifolia, Micromeria fruticosa, Murraya koenigii, Myrrhis odorata, Ocimum basilicum, Ocimum tenuiflorum, Oncorhynchus tshawytscha (Chinook Salmon oil), Origanum vulgare, Perilla frutescens, Petroselinum crispum, Pinus sylvestris, Piper cubeba, Piper nigrum, Pogostemon cablin, Ptychopetalum olacoides, Rosmarinus officinalis, S. aggregatum, Salvia canariensis, Salvia officinalis, Salvia triloba, Schizochytrium sp., Syzygium aromaticum (Eugenia caryophyllata), Tagetes filifolia, Tagetes minuta, Tamarindus indica, Theobroma cacao, Thymus vulgaris, Tripterygium wilfordii, Valeriana officinalis, Vitex agnus-castus, and any combination thereof.

Other endocannabinoid mimetic compounds of the present invention indirectly affect the functioning of the endocannabinoid system by influencing the metabolism (anabolism or catabolism) of the endogenous ligands, anandamide (AEA) or 2-arachidonoylglycerol (2-AG), which in turn interact with CB1 and/or CB2. Enzymes involved in the degradation (catabolism) of AEA include fatty acid amide hydrolase (FAAH), and the synthesis (anabolism) involves N-acyl phosphatidylethanolamine-specific phospholipase D (NAPE-PLD). Enzymes involved in the degradation of 2AG include monacylglycerol lipase (MAGL), and those involved in the synthesis include diacylglycerol lipase 1 and 2 (DAGL1 and DAGL2). Accordingly, certain endocannabinoid mimetic compounds can indirectly affect the functioning of the endocannabinoid system by, e.g., increasing or decreasing the expression of at least one of the following genes: FAAH, NAPE-PLD, MAGL, DAGL1, DAGL2. As shown in FIG. 2 there is a metabolic pathway associated with each shown endocannabinoid, e.g., there is an anandamide and a 2-arachidonoylglycerol metabolic pathway. The former pathway includes NAPE-PLD and FAAH, while the latter pathway includes MAGL, DAGL1, and DAGL2.

In one embodiment, the endocannabinoid mimetic natural extracts that decrease FAAH gene expression include extracts from Aesculus hippocastanum, Allium cepa, Aloe vera, Angelica archangelica, Apium graveolens, Arachis hypogaea, Artemisia annua, Bidens pilosa, Boswellia sacra, Camellia sinensis, Centella asiatica, Cinnamomum verum, Citrus limon, Citrus paradise, Citrus Sinensis, Coleus barbatus, Copaifera officinalis, Coriandrum sativum, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Eugenia caryophyllata, Gallus gallus domesticus (egg oil), Genista tinctorial, Ginkgo biloba, Glycine max, (Soybean), Hedychium flavum, Lindera benzoin, Malus domestica, Matricaria chamomilla, Maytenus chiapensis, Melaleuca alternifolia, Mentha longifolia, Micromeria fruticosa, Murraya koenigii, Myristica fragrans, Myrrhis odorata, Ocimum basilicum, Ocimum tenuiflorum, Origanum vulgare, Panax notogensing (root), Panax gensing, Perilla frutescens, Petroselinum crispum, Pinus sylvestris, Piper cubeba, Piper nigrum, Pogostemon cablin, Ptychopetalum olacoides, Pueraria lobata (Kudzu), Pueraria mirifica (Kwao Krua), Rosmarinus officinalis, S. aggregatum, Salvia canariensis, Salvia officinalis, Salvia triloba, Schizochytrium sp.; Syzygium aromaticum (Eugenia caryophyllata), Tagetes filifolia, Tagetes minuta, Tamarindus indica, Theobroma cacao, Thymus vulgaris, Tripterygium wilfordii, Triticum aestivum, Valeriana officinalis, Vitex agnus-castus, and any combination thereof.

In another embodiment, natural extracts for decreasing FAAH gene expression include extracts from Achyranthes aspera, Aesculus hippocastanum, Aloe vera, Angelica archangelica, Apium graveolens, Arachis hypogaea, Artemisia annua, Bidens pilosa, Boswellia sacra, Camellia sinensis, Centella asiatica, Cinnamomum verum, Citrus limon, Citrus paradise, Coleus barbatus, Copaifera officinalis, Coriandrum sativum, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Eugenia caryophyllata, Gallus gallus domesticus (egg oil), Genista tinctorial, Ginkgo biloba, Glycine max, (Soybean), Hedychium flavum, Lindera benzoin, Malus domestica, Melaleuca alternifolia, Mentha longifolia, Micromeria fruticosa, Murraya koenigii, Myristica fragrans, Myrrhis odorata, Ocimum basilicum, Ocimum tenuiflorum, Origanum vulgare, Panax notogensing (root), Panax gensing, Perilla frutescens, Petroselinum crispum, Pinus sylvestris, Piper cubeba, Piper nigrum, Pogostemon cablin, Ptychopetalum olacoides, Pueraria lobata (Kudzu), Pueraria mirifica (Kwao Krua), Rosmarinus officinalis, Salvia canariensis, Salvia officinalis, Salvia triloba, Syzygium aromaticum (Eugenia caryophyllata), Tagetes filifolia, Tagetes minuta, Tamarindus indica, Theobroma cacao, Thymus vulgaris, Tripterygium wilfordii, Valeriana officinalis, Vitex agnus-castus, and any combination thereof.

In another embodiment, natural extracts for decreasing FAAH gene expression include extracts from Achyranthes aspera, Aesculus hippocastanum, Angelica archangelica, Apium graveolens, Arachis hypogaea, Artemisia annua, Bidens pilosa, Boswellia sacra, Centella asiatica, Cinnamomum verum, Citrus limon, Coleus barbatus, Copaifera officinalis, Coriandrum sativum, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Eugenia caryophyllata, Gallus gallus domesticus (egg oil), Ginkgo biloba, Glycine max, Hedychium flavum, Lindera benzoin, Melaleuca alternifolia, Mentha longifolia, Micromeria fruticosa, Murraya koenigii, Myrrhis odorata, Ocimum basilicum, Ocimum tenuiflorum, Origanum vulgare, Panax notogensing (root), Panax gensing, Perilla frutescens, Petroselinum crispum, Pinus sylvestris, Piper cubeba, Piper nigrum, Pogostemon cablin, Ptychopetalum olacoides, Rosmarinus officinalis, Salvia canariensis, Salvia officinalis, Salvia triloba, Syzygium aromaticum (Eugenia caryophyllata), Tagetes filifolia, Tagetes minuta, Tamarindus indica, Theobroma cacao, Thymus vulgaris, Tripterygium wilfordii, Valeriana officinalis, Vitex agnus-castus, and any combination thereof.

In another embodiment, natural extracts for decreasing FAAH gene expression include extracts from Achyranthes aspera, Aesculus hippocastanum, Angelica archangelica, Apium graveolens, Arachis hypogaea, Artemisia annua, Bidens pilosa, Boswellia sacra, Centella asiatica, Cinnamomum verum, Citrus limon, Clupea pallasii (Pacific Herring oil), Coleus barbatus, Copaifera officinalis, Coriandrum sativum, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Eugenia caryophyllata Euphausia sp. (krill oil), Gallus gallus domesticus (egg oil), Ginkgo biloba, Glycine max, Hedychium flavum, Lindera benzoin, Melaleuca alternifolia, Mentha longifolia, Micromeria fruticosa, Murraya koenigii, Myrrhis odorata, Ocimum basilicum, Ocimum tenuiflorum, Oncorhynchus tshawytscha (Chinook Salmon oil), Origanum vulgare, Panax notogensing (root), Panax gensing, Perilla frutescens, Petroselinum crispum, Pinus sylvestris, Piper cubeba, Piper nigrum, Pogostemon cablin, Ptychopetalum olacoides, Rosmarinus officinalis, S. aggregatum, Salvia canariensis, Salvia officinalis, Salvia triloba, Schizochytrium sp., Syzygium aromaticum (Eugenia caryophyllata), Tagetes filifolia, Tagetes minuta, Tamarindus indica, Theobroma cacao, Thymus vulgaris, Tripterygium wilfordii, Valeriana officinalis, Vitex agnus-castus, and any combination thereof.

In one embodiment, the endocannabinoid mimetic natural extracts that decrease MAGL gene expression include extracts from Achyranthes aspera, Aesculus hippocastanum, Agathosma betulina, Agathosma crenulata, Angelica archangelica, Apium graveolens, Arachis hypogaea, Artemisia annua, Bidens pilosa, Boswellia sacra, Centella asiatica, Cinnamomum verum, Citrus limon, Clupea pallasii (Pacific Herring oil), Coleus barbatus, Copaifera officinalis, Coriandrum sativum, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Eugenia caryophyllata, Euphausia sp. (krill oil), Gallus gallus domesticus (egg oil), Ginkgo biloba, Glycine max, Hedychium flavum, Lindera benzoin, Maytenus chiapensis, Melaleuca alternifolia, Mentha longifolia, Micromeria fruticosa, Murraya koenigii, Myristica fragrans, Myrrhis odorata, Ocimum basilicum, Ocimum tenuiflorum, Oncorhynchus tshawytscha (Chinook Salmon oil), Origanum vulgare, Panax notogensing (root), Panax gensing, Perilla frutescens, Petroselinum crispum, Pinus sylvestris, Piper cubeba, Piper nigrum, Pogostemon cablin, Ptychopetalum olacoides, Rosmarinus officinalis, S. aggregatum, Salvia canariensis, Salvia officinalis, Salvia triloba, Schizochytrium sp., Syzygium aromaticum (Eugenia caryophyllata), Tagetes filifolia, Tagetes minuta, Tamarindus indica, Theobroma cacao, Thymus vulgaris, Tripterygium wilfordii, Valeriana officinalis, Vitex agnus-castus, and any combination thereof.

In another embodiment, natural extracts for decreasing MAGL gene expression include extracts from Aesculus hippocastanum, Angelica archangelica, Apium graveolens, Artemisia annua, Bidens pilosa, Boswellia sacra, Centella asiatica, Cinnamomum verum, Citrus limon, Coleus barbatus, Copaifera officinalis, Coriandrum sativum, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Eugenia caryophyllata, Ginkgo biloba, Hedychium flavum, Lindera benzoin, Maytenus chiapensis, Melaleuca alternifolia, Mentha longifolia, Micromeria fruticosa, Murraya koenigii, Myristica fragrans, Myrrhis odorata, Ocimum basilicum, Ocimum tenuiflorum, Origanum vulgare, Panax notogensing (root), Panax gensing, Perilla frutescens, Petroselinum crispum, Pinus sylvestris, Piper cubeba, Piper nigrum, Pogostemon cablin, Ptychopetalum olacoides, Rosmarinus officinalis, Salvia canariensis, Salvia officinalis, Salvia triloba, Syzygium aromaticum (Eugenia caryophyllata), Tagetes filifolia, Tagetes minuta, Tamarindus indica, Thymus vulgaris, Tripterygium wilfordii, Valeriana officinalis, Vitex agnus-castus, and any combination thereof.

In another embodiment, natural extracts for decreasing MAGL gene expression include extracts from Curcuma longa, Curcuma xanthorrhiza, Curcuma zedoaria, Eugenia caryophyllata, Maytenus chiapensis, Myristica fragrans, Panax notogensing (root), Panax gensing, Tripterygium wilfordii, and any combination thereof.

In another embodiment, natural extracts decreasing MAGL gene expression include extracts from Achyranthes aspera, Aesculus hippocastanum, Agathosma betulina, Agathosma crenulata, Angelica archangelica, Apium graveolens, Arachis hypogaea, Artemisia annua, Bidens pilosa, Boswellia sacra, Centella asiatica, Cinnamomum verum, Citrus limon, Clupea pallasii (Pacific Herring oil), Coleus barbatus, Copaifera officinalis, Coriandrum sativum, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Eugenia caryophyllata Euphausia sp. (krill oil), Gallus gallus domesticus (egg oil), Glycine max, Hedychium flavum, Lindera benzoin, Melaleuca alternifolia, Mentha longifolia, Micromeria fruticosa, Murraya koenigii, Myrrhis odorata, Ocimum basilicum, Ocimum tenuiflorum, Oncorhynchus tshawytscha (Chinook Salmon oil), Origanum vulgare, Panax notogensing (root), Panax gensing, Perilla frutescens, Petroselinum crispum, Pinus sylvestris, Piper cubeba, Piper nigrum, Pogostemon cablin, Ptychopetalum olacoides, Rosmarinus officinalis, S. aggregatum, Salvia canariensis, Salvia officinalis, Salvia triloba, Schizochytrium sp., Syzygium aromaticum (Eugenia caryophyllata), Tagetes filifolia, Tagetes minuta, Tamarindus indica, Theobroma cacao, Thymus vulgaris, Tripterygium wilfordii, Valeriana officinalis, Vitex agnus-castus, and any combination thereof.

The ECS related pathway anti-inflammatory compounds of the present invention can influence inflammatory processes in various fashions. Some anti-inflammatory natural extracts affect a nuclear pathway that increases expression of at least one gene selected from the group consisting of PPARg, PPARa, and PPARb.

In one embodiment, endocannabinoid mimetic natural extracts that increase PPARg gene expression include extracts from Achyranthes aspera, Actinidia deliciosa, Aesculus hippocastanum, Agathosma betulina, Agathosma crenulata, Allium cepa, Allium sativum, Aloe vera, Angelica archangelica, Apium graveolens, Arachis hypogaea, Artemisia annua, Bidens pilosa, Boswellia sacra, Brassica oleracea, Camellia sinensis, Cannabis sativa, Capsicum annuum, Carya illinoinensis, Centella asiatica, Cinnamomum verum, Citrus limon, Citrus paradisi, Citrus Sinensis, Clupea pallasii (Pacific Herring oil), Coleus barbatus, Copaifera officinalis, Coriandrum sativum, Corylus avellane, Cucurbita sp., Daemonorops draco, Dracaena cochinchinensis, Echinacea purpurea, Eucalyptus citriodora, Eucalyptus tetraptera, Eugenia caryophyllata, Euphausia sp. (krill oil), Fragaria ananassa, Gallus gallus domesticus (egg oil), Genista tinctorial, Glycine max (Soybean), Haematococcus pluvialis, Hedychium flavum, Helianthemum glomeratum, Ipomoea batatas, Lindera benzoin, Magnolia biondii, Magnolia dealbata, Magnolia grandiflora, Magnolia obovate, Magnolia officinalis, Malus domestica, Malus domestica, Matricaria chamomilla, Melaleuca alternifolia, Mentha longifolia, Micromeria fruticosa, Murraya koenigii, Myristica fragrans, Myrrhis odorata, Ocimum basilicum, Ocimum tenuiflorum, Oncorhynchus tshawytscha (Chinook Salmon oil), Origanum vulgare, Perilla frutescens, Persea americana, Petroselinum crispum, Pinus sylvestris, Piper cubeba, Piper nigrum, Pistacia vera, Pogostemon cablin, Prunus avium, Prunus persica, Ptychopetalum olacoides, Pueraria lobata (Kudzu), Pueraria mirifica (Kwao Krua), Pyrus sp., Rosmarinus officinalis, Rubus fruticosus, S. aggregatum, Salix alba (bark), Salvia canariensis, Salvia officinalis, Salvia triloba, Schizochytrium sp., Solanum lycopersicum F, Symphytum officinale, Syzygium aromaticum (Eugenia caryophyllata), Tagetes filifolia, Tagetes minuta, Tamarindus indica, Theobroma cacao, Thymus vulgaris, Tripterygium wilfordii, Triticum aestivum, Vaccinium oxycoccos, Valeriana officinalis, Vitex agnus-castus, Vitis vinifera L, and any combination thereof.

In another embodiment, natural extracts for increasing PPARg gene expression include extracts from Achyranthes aspera, Actinidia deliciosa, Agathosma betulina, Agathosma crenulata, Allium sativum, Aloe vera, Arachis hypogaea, Brassica oleracea, Camellia sinensis, Capsicum annuum, Carya illinoinensis, Citrus paradisi, Clupea pallasii (Pacific Herring oil), Corylus avellana, Cucurbita sp., Daemonorops draco, Dracaena cochinchinensis, Eugenia caryophyllata, Euphausia sp. (krill oil), Fragaria ananassa, Gallus gallus domesticus (egg oil), Genista tinctorial, Glycine max (Soybean), Haematococcus pluvialis, Helianthemum glomeratum, Ipomoea batatas, Magnolia biondii, Magnolia dealbata, Magnolia grandiflora, Magnolia obovate, Magnolia officinalis, Malus domestica, Myristica fragrans, Oncorhynchus tshawytscha (Chinook Salmon oil), Origanum vulgare, Perilla frutescens, Persea americana, Pistacia vera, Prunus avium, Prunus persica, Pueraria lobata (Kudzu), Pueraria mirifica (Kwao Krua), Pyrus sp., Rubus fruticosus, S. aggregatum, Salix alba (bark), Schizochytrium sp., Solanum lycopersicum F, Symphytum officinale, Theobroma cacao, Thymus vulgaris, Tripterygium wilfordii, Vaccinium oxycoccos, Vitis vinifera L, and any combination thereof.

In another embodiment, natural extracts for increasing PPARg gene expression include extracts from Achyranthes aspera, Agathosma betulina, Agathosma crenulata, Arachis hypogaea, Clupea pallasii (Pacific Herring oil), Camellia sinensis, Daemonorops draco, Dracaena cochinchinensis, Euphausia sp. (krill oil), Gallus gallus domesticus (egg oil), Glycine max, Magnolia biondii, Magnolia dealbata, Magnolia grandiflora, Magnolia obovate, Magnolia officinalis, Myristica fragrans, Oncorhynchus tshawytscha (Chinook Salmon oil), S. aggregatum, Schizochytrium sp., Theobroma cacao, Tripterygium wilfordii, and any combination thereof.

In another embodiment, natural extracts for increasing PPARg gene expression include extracts from Achyranthes aspera, Agathosma betulina, Agathosma crenulata, Arachis hypogaea, Clupea pallasii (Pacific Herring oil), Daemonorops draco, Dracaena cochinchinensis, Eugenia caryophyllata, Euphausia sp. (krill oil), Gallus gallus domesticus (egg oil), Glycine max, Magnolia biondii, Magnolia dealbata, Magnolia grandiflora, Magnolia obovate, Magnolia officinalis, Myristica fragrans, Oncorhynchus tshawytscha (Chinook Salmon oil), S. aggregatum, Schizochytrium sp., Theobroma cacao, Tripterygium wilfordii, and any combination thereof.

Other endocannabinoid mimetic anti-inflammatory natural extracts suitable for inclusion in compositions of the invention affect an enzymatic pathway that decreases expression of COX1 (i.e., PTGS1), COX2, iNOS, 5-LOX, 12-LOX, a matrix metalloprotease (MMP) or any combination thereof. Examples of such extracts include extracts from Achyranthes aspera, Actinidia deliciosa, Aesculus hippocastanum, Agathosma betulina, Agathosma crenulata, Aleurites moluccana L. Wild (Euphorbiaceae) leaves, Angelica archangelica, Apium graveolens, Arachis hypogaea, Artemisia annua, Artemisia californica, Bidens pilosa, Boswellia sacra, Boswellia serrata, Brassica oleracea, Buddleja officinalis, Camellia sinensis, Cannabis sativa, Capsicum annuum, Carya illinoinensis, Centella asiatica, Cinnamomum verum, Cinnamomum zeylanicum, Citrus limon, Clupea pallasii (Pacific Herring oil), Coleus barbatus, Copaifera officinalis, Coriandrum sativum, Corylus avellana, Cucurbita sp., Curcuma longa, Curcuma xanthorrhiza, Curcuma zedoaria, Daemonorops draco, Dipeptide B-alanine, Dracaena cochinchinensis, Echinacea purpurea, Eucalyptus citriodora, Eucalyptus tetraptera, Eugenia caryophyllata, Euphausia sp. (krill oil), Fragaria ananassa, Gallus gallus domesticus (egg oil), Ginkgo biloba, Glycine max, Glycyrrhiza inflata, Haematococcus pluvialis, Hedychium flavum, Helianthemum glomeratum, Histidine, Ipomoea batatas, Lindera benzoin, Magnolia biondii, Magnolia dealbata, Magnolia grandiflora, Magnolia obovate, Magnolia officinalis, Malus domestica, Matricaria chamomilla, Melaleuca alternifolia, Mentha longifolia, Micromeria fruticosa, Murraya koenigii, Myristica fragrans, Myrrhis odorata, Ocimum basilicum, Ocimum tenuiflorum, Oncorhynchus tshawytscha (Chinook Salmon oil), Origanum vulgare, Panax notogensing (root), Panax gensing, Perilla frutescens f crispa, Perilla frutescens, Persea americana, Petroselinum crispum, Pinus pinaster (bark), Pinus sylvestris, Piper cubeba, Piper nigrum, Pistacia vera, Pogostemon cablin, Prunus avium, Prunus persica, Ptychopetalum olacoides, Pyrus sp., Rosmarinus officinalis, Rubus fruticosus, S. aggregatum, Salvia canariensis, Salvia officinalis, Salvia triloba, Schizochytrium sp., Solanum lycopersicum F, Symphytum officinale, Syzygium aromaticum (Eugenia caryophyllata), Tagetes filifolia, Tagetes minuta, Tamarindus indica, Tanacetum parthenium, Theobroma cacao, Thymus vulgaris, Tripterygium wilfordii, Uncaria tomentosa, Vaccinium oxycoccos, Valeriana officinalis, Vitex agnus-castus, Vitis vinifera L., Zingiber officinale, and any combination thereof.

In another embodiment, natural extracts that decrease expression of COX1 (i.e., PTGS1), COX2, iNOS, 5-LOX, 12-LOX, a matrix metalloprotease (MMP), or any combination thereof are included in compositions of the invention. In aspects, such extracts include extracts from Actinidia deliciosa, Aesculus hippocastanum Angelica archangelica, Apium graveolens, Artemisia annua, Artemisia californica, Bidens pilosa, Boswellia sacra, Boswellia serrata, Buddleja officinalis, Camellia sinensis, Carya illinoinensis, Centella asiatica, Cinnamomum verum, Cinnamomum zeylanicum, Citrus limon, Coleus barbatus, Copaifera officinalis, Coriandrum sativum, Corylus avellana, Curcuma longa, Curcuma xanthorrhiza, Curcuma zedoaria, Eucalyptus citriodora, Eucalyptus tetraptera, Eugenia caryophyllata, Fragaria ananassa, Ginkgo biloba, Hedychium flavum, Helianthemum glomeratum, Lindera benzoin, Malus domestica, Melaleuca alternifolia, Mentha longifolia, Micromeria fruticosa, Murraya koenigii, Myristica fragrans, Myrrhis odorata, Ocimum basilicum, Ocimum tenuiflorum, Origanum vulgare, Panax notogensing (root), Panax gensing, Perilla frutescens f crispa, Perilla frutescens, Persea americana, Petroselinum crispum, Pinus pinaster (bark), Pinus sylvestris, Piper cubeba, Piper nigrum, Pistacia vera, Pogostemon cablin, Prunus avium, Prunus persica, Ptychopetalum olacoides, Pyrus sp., Rosmarinus officinalis, Rubus fruticosus, Salvia canariensis, Salvia officinalis, Salvia triloba, Symphytum officinale, Syzygium aromaticum (Eugenia caryophyllata), Tagetes filifolia, Tagetes minuta, Tamarindus indica, Tanacetum parthenium, Thymus vulgaris, Tripterygium wilfordii, Vaccinium oxycoccos, Valeriana officinalis, Vitex agnus-castus, Zingiber officinale, and any combination thereof.

In another embodiment, natural extracts that decrease expression of COX1 (i.e., PTGS1), COX2, iNOS, 5-LOX, 12-LOX, a matrix metalloprotease (MMP), or any combination thereof are included in compositions. In aspects, such extracts include extracts from Actinidia deliciosa, Aesculus hippocastanum, Angelica archangelica, Apium graveolens, Artemisia annua, Bidens pilosa, Boswellia sacra, Camellia sinensis, Carya illinoinensis, Centella asiatica, Cinnamomum verum, Citrus limon, Coleus barbatus, Copaifera officinalis, Coriandrum sativum, Corylus avellana, Curcuma longa, Curcuma xanthorrhiza, Curcuma zedoaria, Eucalyptus citriodora, Eucalyptus tetraptera, Eugenia caryophyllata, Fragaria ananassa, Ginkgo biloba, Hedychium flavum, Helianthemum glomeratum, Lindera benzoin, Malus domestica, Melaleuca alternifolia, Mentha longifolia, Micromeria fruticosa, Murraya koenigii, Myrrhis odorata, Ocimum basilicum, Ocimum tenuiflorum, Origanum vulgare, Panax notogensing (root), Panax gensing, Perilla frutescens, Persea americana, Petroselinum crispum, Pinus sylvestris, Piper cubeba, Piper nigrum, Pistacia vera, Pogostemon cablin, Prunus avium, Prunus persica, Ptychopetalum olacoides, Pyrus sp., Rosmarinus officinalis, Rubus fruticosus, Salvia canariensis, Salvia officinalis, Salvia triloba, Syzygium aromaticum (Eugenia caryophyllata), Tagetes filifolia, Tagetes minuta, Tamarindus indica, Thymus vulgaris, Tripterygium wilfordii, Vaccinium oxycoccos, Valeriana officinalis, Vitex agnus-castus, and any combination thereof.

In another embodiment, extracts that decrease expression of COX1 (i.e., PTGS1), COX2, iNOS, 5-LOX, 12-LOX, a matrix metalloprotease (MMP), or any combination thereof are included in compositions. In aspects, such extracts include extracts of Aesculus hippocastanum, Agathosma betulina, Agathosma crenulata, Angelica archangelica, Apium graveolens, Arachis hypogaea, Artemisia annua, Bidens pilosa, Boswellia sacra, Centella asiatica, Cinnamomum verum, Citrus limon, Clupea pallasii (Pacific Herring oil), Coleus barbatus, Copaifera officinalis, Coriandrum sativum, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Eugenia caryophyllata, Euphausia sp. (krill oil), Gallus gallus domesticus (egg oil), Ginkgo biloba, Glycine max, Hedychium flavum, Lindera benzoin, Melaleuca alternifolia, Mentha longifolia, Micromeria fruticosa, Murraya koenigii, Myrrhis odorata, Ocimum basilicum, Ocimum tenuiflorum, Oncorhynchus tshawytscha (Chinook Salmon oil), Origanum vulgare, Panax notogensing (root), Panax gensing, Perilla frutescens, Petroselinum crispum, Pinus sylvestris, Piper cubeba, Piper nigrum, Pogostemon cablin, Ptychopetalum olacoides, Rosmarinus officinalis, S. aggregatum, Salvia canariensis, Salvia officinalis, Salvia triloba, Schizochytrium sp., Syzygium aromaticum (Eugenia caryophyllata), Tagetes filifolia, Tagetes minuta, Tamarindus indica, Theobroma cacao, Thymus vulgaris, Tripterygium wilfordii, Valeriana officinalis, Vitex agnus-castus, and any combination thereof.

Still other anti-inflammatory natural extracts suitable for inclusion in the invention affect a cytokine pathway that decreases expression of IL-1beta, IL-1alpha, IL-6, IL-8, NFKB and TNFalpha, increases gene expression in IL10, or results in any combination thereof. In aspects, such extracts include extracts from Achyranthes aspera, Actinidia deliciosa, Aesculus hippocastanum, Agathosma betulina, Agathosma crenulata, Aleurites moluccana L. Wild (Euphorbiaceae) leaves, Allium cepa, Angelica archangelica, Annona cherimola, Apium graveolens, Arachis hypogaea, Artemisia annua, Bidens pilosa, Boswellia sacra, Boswellia serrata, Brassica oleracea, Buddleja officinalis, Calendula officinalis, Camellia sinensis, Capsicum annuum, Carya illinoinensis, Centella asiatica, Cinnamomum verum, Citrus limon, Citrus Sinensis, Clupea pallasii (Pacific Herring oil), Coleus barbatus, Copaifera officinalis, Coriandrum sativum, Corylus avellana, Cucurbita sp., Curcuma longa, Curcuma xanthorrhiza, Curcuma zedoaria, Daemonorops draco, Dipeptide B-alanine, Dracaena cochinchinensis, Echinacea purpurea, Eucalyptus citriodora, Eucalyptus tetraptera, Eugenia caryophyllata, Euphausia sp. (krill oil), Fragaria ananassa, Gallus gallus domesticus (egg oil), Ginkgo biloba, Glycine max, Glycyrrhiza inflata, Haematococcus pluvialis, Hedychium flavum, Helianthemum glomeratum, Histidine, Ipomoea batatas, Lindera benzoin, Magnolia biondii, Magnolia dealbata, Magnolia grandiflora, Magnolia obovate, Magnolia officinalis, Malus domestica, Matricaria chamomilla, Melaleuca alternifolia, Mentha longifolia, Micro HA, Micromeria fruticosa, Murraya koenigii, Myristica fragrans, Myrrhis odorata, Ocimum basilicum, Ocimum tenuiflorum, Oncorhynchus tshawytscha (Chinook Salmon oil), Origanum vulgare, Panax notogensing (root), Panax gensing, Perilla frutescens f crispa, Perilla frutescens, Persea americana, Petroselinum crispum, Pinus sylvestris, Piper cubeba, Piper nigrum, Pistacia vera, Pogostemon cablin, Prunus avium, Prunus persica, Ptychopetalum olacoides, Pyrus sp., Rosmarinus officinalis, Rubus fruticosus, S. aggregatum, Salvia canariensis, Salvia officinalis, Salvia triloba, Schizochytrium sp., Silybum marianum (seed), Solanum lycopersicum F, Symphytum officinale, Syzygium aromaticum (Eugenia caryophyllata), Tagetes filifolia, Tagetes minuta, Tamarindus indica, Tanacetum parthenium, Theobroma cacao, Thymus vulgaris, Tripterygium wilfordii, Triticum aestivum, Uncaria tomentosa, Vaccinium oxycoccos, Valeriana officinalis, Vitex agnus-castus, and any combination thereof.

In another embodiment, one or more natural extracts that decrease expression of IL-1beta, IL-1alpha, IL-6, IL-8, NFKB and TNFalpha, increasing gene expression in IL-10, or any combination thereof, are included in compositions of the invention. In aspects, such extracts include extracts from Achyranthes aspera, Actinidia deliciosa, Aesculus hippocastanum, Agathosma betulina, Agathosma crenulata, Aleurites moluccana L. Wild (Euphorbiaceae) leaves, Allium cepa, Angelica archangelica, Annona cherimola, Apium graveolens, Arachis hypogaea, Artemisia annua, Bidens pilosa, Boswellia sacra, Boswellia serrata, Brassica oleracea, Buddleja officinalis, Calendula officinalis, Camellia sinensis, Capsicum annuum, Carya illinoinensis, Centella asiatica, Cinnamomum verum, Citrus limon, Citrus Sinensis, Clupea pallasii (Pacific Herring oil), Coleus barbatus, Copaifera officinalis, Coriandrum sativum, Corylus avellana, Cucurbita sp., Curcuma longa, Curcuma xanthorrhiza, Curcuma zedoaria, Daemonorops draco, Dipeptide B-alanine, Dracaena cochinchinensis, Echinacea purpurea, Eucalyptus citriodora, Eucalyptus tetraptera, Euphausia sp. (krill oil), Fragaria ananassa, Gallus gallus domesticus (egg oil), Ginkgo biloba, Glycine max, Glycyrrhiza inflata, Haematococcus pluvialis, Hedychium flavum, Helianthemum glomeratum, Histidine, Ipomoea batatas, Lindera benzoin, Magnolia biondii, Magnolia dealbata, Magnolia grandiflora, Magnolia obovata, Magnolia officinalis, Malus domestica, Matricaria chamomilla, Melaleuca alternifolia, Mentha longifolia, Micro HA, Micromeria fruticosa, Murraya koenigii, Myristica fragrans, Myrrhis odorata, Ocimum basilicum, Ocimum tenuiflorum, Oncorhynchus tshawytscha (Chinook Salmon oil), Origanum vulgare, Panax notogensing (root), Panax gensing, Perilla frutescens f crispa, Perilla frutescens, Persea americana, Petroselinum crispum, Pinus sylvestris, Piper cubeba, Piper nigrum, Pistacia vera, Pogostemon cablin, Prunus avium, Prunus persica, Ptychopetalum olacoides, Pyrus sp., Rosmarinus officinalis, Rubus fruticosus, S. aggregatum, Salvia canariensis, Salvia officinalis, Salvia triloba, Schizochytrium sp., Silybum marianum (seed), Solanum lycopersicum F, Symphytum officinale, Syzygium aromaticum (Eugenia caryophyllata), Tagetes filifolia, Tagetes minuta, Tamarindus indica, Tanacetum parthenium, Theobroma cacao, Thymus vulgaris, Tripterygium wilfordii, Triticum aestivum, Uncaria tomentosa, Vaccinium oxycoccos, Valeriana officinalis, Vitex agnus-castus, and any combination thereof.

In another embodiment, one or more natural extracts that decrease expression of IL-1beta, IL-1alpha, IL-6, IL-8, NFKB and TNFalpha, increasing gene expression in IL-10, or any combination thereof, are incorporated in compositions of the invention. In aspects, such extracts include extracts from Achyranthes aspera, Actinidia deliciosa, Aesculus hippocastanum, Agathosma betulina, Agathosma crenulata, Allium cepa, Angelica archangelica, Apium graveolens, Arachis hypogaea, Artemisia annua, Bidens pilosa, Boswellia sacra, Calendula officinalis, Camellia sinensis, Carya illinoinensis, Centella asiatica, Cinnamomum verum, Citrus limon, Citrus Sinensis, Clupea pallasii (Pacific Herring oil), Coleus barbatus, Copaifera officinalis, Coriandrum sativum, Corylus avellana, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Euphausia sp. (krill oil), Fragaria ananassa, Gallus gallus domesticus (egg oil), Ginkgo biloba, Glycine max, Hedychium flavum, Helianthemum glomeratum, Lindera benzoin, Magnolia biondii, Magnolia dealbata, Magnolia grandiflora, Magnolia obovata, Magnolia officinalis, Malus domestica, Matricaria chamomilla, Melaleuca alternifolia, Mentha longifolia, Micromeria fruticosa, Murraya koenigii, Myristica fragrans, Myrrhis odorata, Ocimum basilicum, Ocimum tenuiflorum, Oncorhynchus tshawytscha (Chinook Salmon oil), Origanum vulgare, Panax notogensing (root), Panax gensing, Perilla frutescens, Persea americana, Petroselinum crispum, Pinus sylvestris, Piper cubeba, Piper nigrum, Pistacia vera, Pogostemon cablin, Prunus avium, Prunus persica, Ptychopetalum olacoides, Pyrus sp., Rosmarinus officinalis, Rubus fruticosus, S. aggregatum, Salvia canariensis, Salvia officinalis, Salvia triloba, Schizochytrium sp., Syzygium aromaticum (Eugenia caryophyllata),Tagetes filifolia, Tagetes minuta, Tamarindus indica, Theobroma cacao, Thymus vulgaris, Tripterygium wilfordii, Triticum aestivum, Vaccinium oxycoccos, Valeriana officinalis, Vitex agnus-castus, and any combination thereof.

In another embodiment, one or more natural extracts that decrease gene expression in IL-1a are included in compositions of the invention. In aspects, such extracts include extracts of Achyranthes aspera, Actinidia deliciosa, Aesculus hippocastanum, Agathosma betulina, Agathosma crenulata, Allium cepa, Angelica archangelica, Apium graveolens, Arachis hypogaea, Artemisia annua, Bidens pilosa, Boswellia sacra, Camellia sinensis, Carya illinoinensis, Centella asiatica, Cinnamomum verum, Citrus limon, Citrus Sinensis, Clupea pallasii (Pacific Herring oil), Coleus barbatus, Copaifera officinalis, Coriandrum sativum, Corylus avellana, Curcuma longa, Curcuma xanthorrhiza, Curcuma zedoaria, Daemonorops draco, Dracaena cochinchinensis, Eucalyptus citriodora, Eucalyptus tetraptera, Euphausia sp. (krill oil), Fragaria ananassa, Gallus gallus domesticus (egg oil), Ginkgo biloba, Glycine max, Hedychium flavum, Helianthemum glomeratum, Lindera benzoin, Magnolia biondii, Magnolia dealbata, Magnolia grandiflora, Magnolia obovata, Magnolia officinalis, Malus domestica, Matricaria chamomilla, Melaleuca alternifolia, Mentha longifolia, Micromeria fruticosa, Murraya koenigii, Myristica fragrans, Myrrhis odorata, Ocimum basilicum, Ocimum tenuiflorum, Oncorhynchus tshawytscha (Chinook Salmon oil), Origanum vulgare, Panax notogensing (root), Panax gensing, Perilla frutescens, Persea americana, Petroselinum crispum, Pinus sylvestris, Piper cubeba, Piper nigrum, Pistacia vera, Pogostemon cablin, Prunus avium, Prunus persica, Ptychopetalum olacoides, Pyrus sp., Rosmarinus officinalis, Rubus fruticosus, S. aggregatum, Salvia canariensis, Salvia officinalis, Salvia triloba, Schizochytrium sp., Syzygium aromaticum (Eugenia caryophyllata), Tagetes filifolia, Tagetes minuta, Tamarindus indica, Theobroma cacao, Thymus vulgaris, Tripterygium wilfordii, Triticum aestivum, Vaccinium oxycoccos, Valeriana officinalis, Vitex agnus-castus, and any combination thereof.

In another embodiment, one or more natural extracts that decrease NFKB gene expression are included in compositions. In aspects, such extracts include extracts from Achyranthes aspera, Actinidia deliciosa, Aesculus hippocastanum, Agathosma betulina, Agathosma crenulata, Angelica archangelica, Apium graveolens, Arachis hypogaea, Artemisia annua, Bidens pilosa, Boswellia sacra, Calendula officinalis, Camellia sinensis, Carya illinoinensis, Centella asiatica, Cinnamomum verum, Citrus limon, Clupea pallasii (Pacific Herring oil), Coleus barbatus, Copaifera officinalis, Coriandrum sativum, Corylus avellana, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Euphausia sp. (krill oil), Fragaria ananassa, Gallus gallus domesticus (egg oil), Ginkgo biloba, Glycine max, Hedychium flavum, Helianthemum glomeratum, Lindera benzoin, Malus domestica, Melaleuca alternifolia, Mentha longifolia, Micromeria fruticosa, Murraya koenigii, Myrrhis odorata, Ocimum basilicum, Ocimum tenuiflorum, Oncorhynchus tshawytscha (Chinook Salmon oil), Origanum vulgare, Perilla frutescens, Persea americana, Petroselinum crispum, Pinus sylvestris, Piper cubeba, Piper nigrum, Pistacia vera, Pogostemon cablin, Prunus avium, Prunus persica, Ptychopetalum olacoides, Pyrus sp., Rosmarinus officinalis, Rubus fruticosus, S. aggregatum, Salvia canariensis, Salvia officinalis, Salvia triloba, Schizochytrium sp., Syzygium aromaticum (Eugenia caryophyllata), Tagetes filifolia, Tagetes minuta, Tamarindus indica, Theobroma cacao, Thymus vulgaris, Tripterygium wilfordii, Vaccinium oxycoccos, Valeriana officinalis, Vitex agnus-castus, and any combination thereof.

In another embodiment, one or more natural extracts that increase expression of TRPA1 or TRPM8, decreasing expression of TRPV1, or any combination thereof are included in compositions. In aspects, such extracts include extracts from Achyranthes aspera, Aesculus hippocastanum, Agathosma betulina, Agathosma crenulata, Allium sativum, Angelica archangelica, Apium graveolens, Arachis hypogaea, Artemisia annua, Artemisia californica, Bidens pilosa, Boswellia sacra, Boswellia serrata, Brassica juncea, Brassica nigra, Brassica oleracea, Capsicum annuum, Centella asiatica, Cinnamomum camphora, Cinnamomum verum, Cinnamomum zeylanicum, Citrus bergamia, Citrus limon, Clupea pallasii (Pacific Herring oil), Coleus barbatus, Copaifera officinalis, Coriandrum sativum, Curcuma longa, Curcuma xanthorrhiza, Curcuma zedoaria, Echinacea purpurea, Eucalyptus citriodora, Eucalyptus tetraptera, Eugenia caryophyllata, Euphausia sp. (krill oil), Gallus gallus domesticus (egg oil), Gaultheria procumbens, Ginkgo biloba, Glycine max, Hedychium flavum, Lindera benzoin, Magnolia biondii, Magnolia dealbata, Magnolia grandiflora, Magnolia obovate, Magnolia officinalis, Matricaria chamomilla, Melaleuca alternifolia, Melaleuca leucadendra L., Mentha longifolia, Mentha spicata, Micromeria fruticosa, Murraya koenigii, Myristica fragrans, Myrrhis odorata, Ocimum basilicum L., Ocimum tenuiflorum, Oncorhynchus tshawytscha (Chinook Salmon oil), Origanum vulgare, Panax notogensing (root), Panax gensing, Perilla frutescens, Petroselinum crispum, Pinus sylvestris, Piper cubeba, Piper nigrum, Pogostemon cablin, Ptychopetalum olacoides, Rosmarinus officinalis, S. aggregatum, Salix alba (bark),Salvia canariensis, Salvia mellifera, Salvia officinalis, Salvia triloba, Schizochytrium sp., Solanum melongena, Syzygium aromaticum (Eugenia caryophyllata), Tagetes filifolia, Tagetes minuta, Tamarindus indica, Tanacetum parthenium, Theobroma cacao, Thymus vulgaris, Tripterygium wilfordii, Valeriana officinalis, Vanilla planifolia, Vitex agnus-castus, Wasabia japonica Root, Zanthoxylum americanum, Zingiber officinale, and any combination thereof.

In another embodiment, one or more natural extracts increasing expression of TRPA1 or TRPM8, decreasing expression of TRPV1, or any combination thereof, are included in compositions. In aspects, such extracts include extracts from Achyranthes aspera, Aesculus hippocastanum, Agathosma betulina, Agathosma crenulata, Angelica archangelica, Apium graveolens, Arachis hypogaea, Artemisia annua, Artemisia californica, Bidens pilosa, Boswellia sacra, Boswellia serrata, Brassica juncea, Brassica nigra, Capsicum annuum, Centella asiatica, Cinnamomum verum, Cinnamomum zeylanicum, Citrus bergamia, Citrus limon, Clupea pallasii (Pacific Herring oil), Coleus barbatus, Copaifera officinalis, Coriandrum sativum, Curcuma longa, Curcuma xanthorrhiza, Curcuma zedoaria, Echinacea purpurea, Eucalyptus citriodora, Eucalyptus tetraptera, Eugenia caryophyllata, Euphausia sp. (krill oil), Gallus gallus domesticus (egg oil), Gaultheria procumbens, Ginkgo biloba, Glycine max, Hedychium flavum, Lindera benzoin, Magnolia biondii, Magnolia dealbata, Magnolia grandiflora, Magnolia obovate, Magnolia officinalis, Melaleuca alternifolia, Melaleuca leucadendra L., Mentha longifolia, Mentha spicata, Micromeria fruticosa, Murraya koenigii, Myristica fragrans, Myrrhis odorata, Ocimum basilicum L., Ocimum tenuiflorum, Oncorhynchus tshawytscha (Chinook Salmon oil), Origanum vulgare, Panax notogensing (root), Panax gensing, Perilla frutescens, Petroselinum crispum, Pinus sylvestris, Piper cubeba, Piper nigrum, Pogostemon cablin, Ptychopetalum olacoides, Rosmarinus officinalis, S. aggregatum, Salix alba (bark),Salvia canariensis, Salvia mellifera, Salvia officinalis, Salvia triloba, Schizochytrium sp., Solanum melongena, Syzygium aromaticum (Eugenia caryophyllata), Tagetes filifolia, Tagetes minuta, Tamarindus indica, Tanacetum parthenium, Theobroma cacao, Thymus vulgaris, Tripterygium wilfordii, Valeriana officinalis, Vanilla planifolia, Vitex agnus-castus, Wasabia japonica Root, Zanthoxylum americanum, Zingiber officinale, and any combination thereof.

In another embodiment, one or more natural extracts that increase expression of TRPA1 or TRPM8, decreasing expression of TRPV1, or any combination thereof, are incorporated in compositions of the invention. In aspects, such extracts include extracts from Achyranthes aspera, Aesculus hippocastanum, Agathosma betulina, Agathosma crenulata, Angelica archangelica, Apium graveolens, Arachis hypogaea, Artemisia annua, Bidens pilosa, Boswellia sacra, Centella asiatica, Cinnamomum camphora, Cinnamomum verum, Citrus limon, Clupea pallasii (Pacific Herring oil), Coleus barbatus, Copaifera officinalis, Coriandrum sativum, Curcuma longa, Curcuma xanthorrhiza, Curcuma zedoaria, Echinacea purpurea, Eucalyptus citriodora, Eucalyptus tetraptera, Eugenia caryophyllata, Euphausia sp. (krill oil), Gallus gallus domesticus (egg oil), Gaultheria procumbens, Ginkgo biloba, Glycine max, Hedychium flavum, Lindera benzoin, Magnolia biondii, Magnolia dealbata, Magnolia grandiflora, Magnolia obovata, Magnolia officinalis, Melaleuca alternifolia, Mentha longifolia, Micromeria fruticosa, Murraya koenigii, Myristica fragrans, Myrrhis odorata, Ocimum basilicum, Ocimum tenuiflorum, Oncorhynchus tshawytscha (Chinook Salmon oil), Origanum vulgare, Panax notogensing (root), Panax gensing, Perilla frutescens, Petroselinum crispum, Pinus sylvestris, Piper cubeba, Piper nigrum, Pogostemon cablin, Ptychopetalum olacoides, Rosmarinus officinalis, S. aggregatum, Salvia canariensis, Salvia officinalis, Salvia triloba, Schizochytrium sp., Syzygium aromaticum (Eugenia caryophyllata), Tagetes filifolia, Tagetes minuta, Tamarindus indica, Theobroma cacao, Thymus vulgaris, Tripterygium wilfordii, Valeriana officinalis, Vitex agnus-castus, and any combination thereof.

In another embodiment, one or more natural extracts that increase expression of TRPA1 are incorporated in compositions. In aspects, such extracts include extracts from Achyranthes aspera, Arachis hypogaea, Cinnamomum camphora, Clupea pallasii (Pacific Herring oil), Curcuma longa, Curcuma xanthorrhiza, Curcuma zedoaria, Echinacea purpurea, Euphausia sp. (krill oil), Eugenia caryophyllata, Gallus gallus domesticus (egg oil), Gaultheria procumbens, Ginkgo biloba, Glycine max, Magnolia biondii, Magnolia dealbata, Magnolia grandiflora, Magnolia obovate, Magnolia officinalis, Mentha longifolia, Myristica fragrans, Oncorhynchus tshawytscha (Chinook Salmon oil), Panax notogensing (root), Panax gensing, S. aggregatum, Schizochytrium sp., Theobroma cacao, Tripterygium wilfordii, and any combination thereof.

In another embodiment, one or more natural extracts that increase expression of TRPM8 are in compositions. In aspects, such extracts include extracts from Aesculus hippocastanum, Agathosma betulina, Agathosma crenulata, Angelica archangelica, Apium graveolens, Arachis hypogaea, Artemisia annua, Bidens pilosa, Boswellia sacra, Centella asiatica, Cinnamomum verum, Citrus limon, Clupea pallasii (Pacific Herring oil), Coleus barbatus, Copaifera officinalis, Coriandrum sativum, Curcuma longa, Curcuma xanthorrhiza, Curcuma zedoaria, Eucalyptus citriodora, Eucalyptus tetraptera, Eugenia caryophyllata, Euphausia sp. (krill oil), Gallus gallus domesticus (egg oil), Ginkgo biloba, Glycine max, Hedychium flavum, Lindera benzoin, Magnolia biondii, Magnolia dealbata, Magnolia grandiflora, Magnolia obovate, Magnolia officinalis, Melaleuca alternifolia, Mentha longifolia, Micromeria fruticosa, Murraya koenigii, Myrrhis odorata, Ocimum basilicum, Ocimum tenuiflorum, Oncorhynchus tshawytscha (Chinook Salmon oil), Origanum vulgare, Perilla frutescens, Petroselinum crispum, Pinus sylvestris, Piper cubeba, Piper nigrum, Pogostemon cablin, Ptychopetalum olacoides, Rosmarinus officinalis, S. aggregatum, Salvia canariensis, Salvia officinalis, Salvia triloba, Schizochytrium sp., Syzygium aromaticum (Eugenia caryophyllata), Tagetes filifolia, Tagetes minuta, Tamarindus indica, Thymus vulgaris, Tripterygium wilfordii, Valeriana officinalis, Vitex agnus-castus, and any combination thereof.

In another embodiment, one or more natural extracts that decrease expression of TRPV1 are included in compositions. In aspects, such extracts include extracts from Aesculus hippocastanum, Agathosma betulina, Agathosma crenulata Angelica archangelica, Apium graveolens, Artemisia annua, Bidens pilosa, Boswellia sacra, Centella asiatica, Cinnamomum verum, Citrus limon, Coleus barbatus, Copaifera officinalis, Coriandrum sativum, Eucalyptus citriodora, Eucalyptus tetraptera, Hedychium flavum, Lindera benzoin, Melaleuca alternifolia, Mentha longifolia, Micromeria fruticosa, Murraya koenigii, Myrrhis odorata, Ocimum basilicum, Ocimum tenuiflorum, Origanum vulgare, Perilla frutescens, Petroselinum crispum, Pinus sylvestris, Piper cubeba, Piper nigrum, Pogostemon cablin, Ptychopetalum olacoides, Rosmarinus officinalis, Salvia canariensis, Salvia officinalis, Salvia triloba, Syzygium aromaticum (Eugenia caryophyllata), Tagetes filifolia, Tagetes minuta, Tamarindus indica, Thymus vulgaris, Valeriana officinalis, Vitex agnus-castus, and any combination thereof.

In another embodiment, one or more natural extracts that increase expression of TRPA1 or TRPM8, decrease expression of TRPV1, or result in any combination thereof, are included in compositions. In aspects, such extracts include extracts from Achyranthes aspera, Aesculus hippocastanum, Agathosma betulina, Agathosma crenulata, Angelica archangelica, Apium graveolens, Arachis hypogaea, Artemisia annua, Bidens pilosa, Boswellia sacra, Centella asiatica, Cinnamomum verum, Citrus limon, Clupea pallasii (Pacific Herring oil), Coleus barbatus, Copaifera officinalis, Coriandrum sativum, Curcuma longa, Curcuma xanthorrhiza, Curcuma zedoaria, Daemonorops draco, Dracaena cochinchinensis, Echinacea purpurea, Eucalyptus citriodora, Eucalyptus tetraptera, Euphausia sp. (krill oil), Gallus gallus domesticus (egg oil), Glycine max, Hedychium flavum, Lindera benzoin, Melaleuca alternifolia, Mentha longifolia, Micromeria fruticosa, Murraya koenigii, Myrrhis odorata, Ocimum basilicum, Ocimum tenuiflorum, Oncorhynchus tshawytscha (Chinook Salmon oil), Origanum vulgare, Perilla frutescens, Petroselinum crispum, Pinus sylvestris, Piper cubeba, Piper nigrum, Pogostemon cablin, Ptychopetalum olacoides, Rosmarinus officinalis, S. aggregatum, Salvia canariensis, Salvia officinalis, Salvia triloba, Schizochytrium sp., Syzygium aromaticum (Eugenia caryophyllata), Tagetes filifolia, Tagetes minuta, Tamarindus indica, Theobroma cacao, Thymus vulgaris, Tripterygium wilfordii, Valeriana officinalis, and Vitex agnus-castus.

Exemplary chemical classes, compounds, and natural extracts for modulating desired beneficial gene expression impacting the four major ECS and related pathways, i.e.: (1) direct ECS receptors, (2) indirect ECS receptors, (3) inflammatory related pathways (nuclear, enzymatic, cytokine), and (4) ECS related TRP pathways, are summarized in tables 5, 6, and 7.

In aspects, compositions can be characterized based on the ratio of the amounts of the compounds and/or natural extracts within a single composition. In certain embodiments, compositions containing three natural extracts and/or compounds, corresponding the four main pathways of the invention, are provided, wherein the ratios of direct endocannabinoid compound: indirect endocannabinoid compound: ECS related pathway anti-inflammatory: ECS related TRP pathway compound by weight, are in a ratio of about 1:1:1:1, >1:1:1:1, 1:>1:1:1, 1:1:>1:1, 1:1:1:>1, 1:>1:>1:1, 1::>1:>1:1, 1:1:>1:>1, >1:1:1>1, >1:1:>1:1, 1:>1:1:>1, >1:>1:>1:1, 1:>:1:>1:>1, >1:>1:1:>1, >1:1:>1:>1 wherein in some embodiments the ratio is <1: >1:1:>1, 1:>1 >1:>1, >1:>1:1:>1, or <1:<1:<1:1, to each other to maximize beneficial effect. “About” is applicable to all listed ratios, and any number defined in these ratios can be approximate or exact.

In some embodiments, the composition further includes at least one cannabinoid compound. Cannabinoid compounds can be collected from any part of a suitable cannabis plant, including leaf, including fan and sugar leaves, bract, trichomes, cola, flower, husk, stem, and node. The cannabinoids can be present in the compositions of the invention as part of a cannabinoid-containing extract. Preferred embodiments of cannabinoid-containing extracts include hemp seed oil, and extracts of Cannabis sativa, including husk, stem, flower, bract, and leaf extracts.

In some embodiments, the compositions include cannabidiol, preferably contained within a hemp seed oil extract. In such embodiments, the compositions preferably include cannabidiol at a concentration of 200 ppm or less, 150 ppm or less, 100 ppm or less, 75 ppm or less, 50 ppm or less, or 25 ppm or less. In other embodiments, the cannabidiol is contained in a flower or leaf extract of Cannabis sativa, in which case the concentration of cannabidiol is more than 25 ppm, more than 50 ppm, more than 75 ppm, more than 100 ppm, more than 150 ppm, or more than 200 ppm.

Cosmetic, dermatological and/or pharmaceutical compositions of the invention typically contain any suitable effective amount, based on the total weight of the composition, of the endocannabinoid mimetic compounds and/or natural extract blend. An effective amount can be measured by, for instance, in-vitro and in-vivo physiological results, including topical in-vivo efficacy and ex-vivo cell modeling efficacy, including standards for both cosmetic and pharmaceutical effects. In some aspects, the suitable amount is about 0.001% to about 30%, based on the total weight of the composition, of the endocannabinoid mimetic compounds and/or natural extract blend. Preferably the amount of compound and/or natural extract blend of the invention included in the cosmetic and/or dermatological compositions of the invention is about 0.01% to about 25%, about 0.05% to about 10%, about 0.1% to about 5% (e.g., 7.0 wt. %), about 5% to about 25%. Cosmetic, therapeutic (e.g., dermatological), and pharmaceutical compositions of the invention typically contain about 0.0001% to about 10%, based on the total weight of the composition, of each endocannabinoid mimetic compound and/or natural extract present within the endocannabinoid mimetic compound and/or natural extract blend. In aspects, the amount of each endocannabinoid mimetic compound and/or natural extract present within the endocannabinoid mimetic compound and/or natural extract blend is about 0.0001%-10%, 0.0001%-5%, 0.0001%-2%, 0.0001%-0.5%, 0.0001%-0.05%, 0.001%-5%, 0.001%-2%, 0.001%-0.5%, 0.001%-0.05%, 0.01%-5%, 0.01%-2%, 0.01%-0.5%, and 0.01%-0.05%.

The endocannabinoid mimetic compound and/or natural extract blend of the invention (and compositions discussed herein containing the same) can be used to treat or prevent a wide variety of skin changes, including skin changes resulting from intrinsic and extrinsic aging. For example, a composition containing the endocannabinoid mimetic compound and/or extract blend of the invention can be used to treat or reduce visible signs of intrinsic and/or extrinsic aging such as skin wrinkling and/or fine lines, skin sagging, skin dryness, skin thinness and/or transparency, skin firmness, skin smoothness, uneven texture, nail plate thinning and/or ridging, and the like. In addition, a composition containing the endocannabinoid mimetic compound and/or natural extract blend of the invention can be used to treat or prevent erythematous; inflammatory, allergic, or autoimmune-reactive symptoms, in particular dermatoses; skin changes in light-sensitive skin, particularly photodermatoses; and damaging effects of the UV part of solar radiation on the skin such as skin blotchiness and/or darkening, age spots, spider veins, actinic keratoses, and the like. The compounds of the invention can further be used to treat melasma and post inflammatory hyperpigmentation (PIH).

Thus the invention is also directed to a method of treating or preventing the worsening or otherwise modulating the course and/or severity of a skin change (e.g., acne, skin aging (e.g., wrinkling, fine lines), pigment dyschromia, including hyperpigmentation (e.g., age spots), UV damage, erythema, cellulitis, rosacea, eczema, dermatitis (atopic or contact or non-specific), pruritis, lupus, acne, keratosis pilaris, actinic keratosis, seborrheic keratosis and other inflammatory skin conditions, photodamage, photoreactions, acute burns (thermal, sunburn/UV, radiation, etc.) comprising topically administering a composition comprising the endocannabinoid mimetic compound and/or natural extract blend.

Embodiments of the endocannabinoid mimetic compounds and/or natural extracts (and compositions containing the same) may be used in an effective amount, an effective number of times, over an effective course of treatment, to treat autoimmune disorders (e.g. lupus, vitiligo, pemphigus, pemphigoid, scleroderma, vasculitis) and used to treat inflammation including acute inflammation (for example sunburn or thermal burn, dermatitis (atopic, contact, allergic or non-specific), and many other ‘-itis’ (e.g. pruritus, (itch), seborrheic dermatitis, cellulitis) which are inflammatory in nature) or chronic inflammation (for example eczema, psoriasis, rosacea, as well as inflammatory inherited disorders and many others), to mitigate proinflammatory responses caused by topical skincare ingredients (e.g. low molecular weight HA, high concentration and low pH AHAs, retinoids) and to treat immune dysfunction, wound healing especially abnormal wound healing, incompletely repaired inflammatory damage from UV light resulting in microinjuries that over time may accumulate to create wrinkles, pigment problems, telangiectatic blood vessels, loss of collagen, elastin, hyaluronic acid as well as mitochondrial damage.

In addition, embodiments of the endocannabinoid mimetic compound and/or natural extract blend of the invention (and compositions containing the same) can be used to promote cosmetic skin changes that improve the appearance of skin, including skin tightening, skin brightening, skin illuminating, skin smoothing, skin moistening, skin plumping, skin firming, evening of skin tone, reducing skin redness, minimizing the appearance of dark circles, improving skin elasticity and recoilability, improving overall skin cell health, reducing pore size, and reducing the appearance of fine lines, wrinkles and skin blemishes resulting from acne or aging.

Compositions containing the endocannabinoid mimetic compound and/or natural extract blend of the present invention can be used to reduce, if not completely prevent, damage to the skin caused by any imbalance in the ECS pathway homeostasis pathway (e.g. immune response and inflammation/oxidative stress, pain and itch response, skin matrices modulation, apoptosis and senescence, barrier function and lipid synthesis, pigmentation) (or) used to promote prejuvenation, rejuvenation or regeneration of any dysfunctional ECS influenced target tissue or cell type (e.g. keratinocytes, fibroblasts, melanocytes, sebocytes, adipocytes, langerhans cells, dermal papillae cells, dendritic cells, macrophages, mast cells, various T cell populations and also endothelial and vascular cells, and merkle cells). Furthermore, such endocannabinoid mimetic compositions can be used to maintain ECS healthy homeostatic pathways and cell types.

Furthermore, embodiments of the endocannabinoid mimetic compound and/or natural extract blend of the invention (and compositions containing the same) can be used to treat, reduce, or prevent burns caused by UVA, UVB, visible light, HEV, blue/violet light, IRA, therapeutic ionizing radiation, and thermal and chemical sources.

Yet further, embodiments of the endocannabinoid mimetic compound and/or natural extracts blend of the invention (and compositions containing the same) can be used to maintain or improve the barrier function of the skin, by stimulating production of epidermal lipids such as epidermal ceramides, which function as structural components for the stratum corneum. The increased ceramide presence can also induce production of anti-microbial peptides, further improving and/or re-establishing homeostasis in skin microbiome.

Still further, a blend of the endocannabinoid mimetic compounds and/or natural extracts of the invention (and compositions containing the same) can be used to treat skin matrix dysfunction, where injury or damage of the effects of intrinsic and extrinsic aging manifest, including inducing an improvement in the extracellular matrix, improvement in cellular, cellular proliferation, differentiation, autophagy, apoptosis, and senescence. Skin matrix functionality is highly influenced by ECS homeostasis via CERS, AP1, FLG, CASP8, MAPK/ERK pathways.

Still further, a blend of the endocannabinoid mimetic compounds and/or natural extracts of the invention (and compositions containing the same) can be used to treat inflammatory skin responses and improve wound healing. This particularly important post medical procedures such as laser, microdermabrasion, chemical peels, micro-needling, injectable fillers and toxins, medical/surgical or non-medical procedures which produce inflammation and/or a wound including laser, intense pulsed light radio frequency, ultrasound, microwave, plasma, chemical peels, microdermabrasion, injectable fillers or toxins, and micro-needling.

Still further a blend of the endocannabinoid mimetic compounds and/or natural extracts of the invention (and compositions containing the same) can be used to reduce, diminish, repair cellular or premature senescence by either reversing the cell's senescent state or eliminating a cell before it enters a senescent state through apoptosis; the result being an improvement in longevity of the host of the cell type; and therefore used to prevent or mitigate skin carcinogenesis and damage to stem cells and impaired regenerative function.

Still further a blend of the endocannabinoid mimetic compounds and/or natural extracts of the invention (and compositions containing the same) can be used to reduce, diminish, repair inflammatory and non-inflammatory lesions characteristic with acne, and to promote a clearing and normalization of blemish prone skin.

Still further, a blend of the endocannabinoid mimetic compounds and/or natural extracts of the invention (and compositions containing the same) can be used to treat the detrimental effects of disease (e.g. psoriasis) or genetic disorders (e.g. progeria) whereby such conditions are at least in part modulated by the human endocannabinoid system.

Still further, embodiments of the endocannabinoid mimetic compounds and/or natural extracts of the invention (and compositions containing the same) can be used to provide pain relief and treat arthritis, such as rheumatoid arthritis, and other inflammatory conditions, including joint and skeletal muscle pain. e.g., by affecting the activity of nocioceptors to block or reduce the transmission of pain signals. Types of pain which are reduced by the pain-reducing compounds and/or extracts disclosed in the invention by means of gene modulation of the ionotropic pain pathways described hereunder include nociceptive pain, neuropathic pain, visceral pain, and combinations thereof. Pain-reducing compounds suitable for inclusion in the compositions of the invention preferably reduce, either directly or indirectly, nociceptive pain. In some cases, the pain-reducing compounds reduce nociceptive pain by being a counter-irritant, thereby reducing or eliminating the transmission of pain. Nociceptive pain includes pain elicited when noxious stimuli such as inflammatory chemical mediators are released following tissue injury, disease, or inflammation and are detected by normally functioning sensory receptors (nociceptors) at the site of injury. Examples of nociceptive pain include pain associated with chemical and thermal burns, burn from electromagnetic radiation, including burns from UVA, UVB, visible light, HEV, blue/violet light, IRA, and therapeutic ionizing radiation), cuts and contusions of the skin, osteoarthritis, rheumatoid arthritis, tendonitis, and myofascial pain.

The invention provides, in aspects, diverse blends of endocannabinoid mimetic compounds and/or natural extracts, and cosmetic and/or dermatological compositions containing such blends, that reduce skin changes that result in unhealthy or unattractive skin, e.g., by providing a positive functional impact on an ECS direct, indirect or related pathway via a positive impact on an ECS influenced cell type. In addition, or alternatively, the present invention pertains to blends of endocannabinoid mimetic compounds and/or natural extracts, and cosmetic, pharmaceutical, and/or dermatological compositions containing such blends, that promote skin changes that improve skin health or appearance, e.g., by providing the dermal and/or epidermal cells a friendly environment in which to undergo natural skin repair processes.

Subjects appropriate for treatment with the compositions of the invention include mammals, such as, but not limited to, humans, pigs, dogs, cats, cows, goats, sheep, and horses. Preferably the subject is human.

Still further, a blend of the endocannabinoid mimetic compounds and/or natural extracts of the invention (and compositions containing the same) can be delivered by topical or systemic routes of administration.

Cosmetic, pharmaceutical, and/or therapeutic drug dermatological compositions of the invention can be topically applied to the skin, hair or scalp, by any suitable method, including, but not limited to, injection or micro-needling, transdermal patch, decoys, ultrasonic delivery, and laser assisted delivery.

The compositions of the present invention typically contain at least one additive. Suitable additives include, but are not limited to, surfactants, cosmetic auxiliaries, pigments, UVA filters, UVB filters, visible light filters, HEV filters, blue/violet filters, IRA filters, skin absorption promoting agents, propellants, thickening agents, emulsifiers, solvents (e.g., alcoholic solvents), water, perfumes, dyestuffs, deodorants, antimicrobial materials, back-fatting agents, complexing and sequestering agents, exfoliating agents, pearlescent agents, plant extracts, skin condition dependent cosmetic quasi active or therapeutic drug active ingredients, and/or derivatives and combinations thereof.

The compositions of the invention optionally further comprise substances which absorb, scatter, reflect, or block electromagnetic radiation in the UVB, UVA, HEV and IR range, wherein the total quantity of filter substances is, for example 0.1 wt % to 40 wt %, preferably 0.5 to 20 wt %, more preferably 1.0 to 15.0 wt %, based on the total weight of the compositions, in order to provide cosmetic compositions which protect the skin from the entire range of ultraviolet radiation and serve as sunscreen agents for the skin. Suitable filter substances can be either oil-soluble or water-soluble, either chemical (e.g. octyl methoxycinnamate) or physical (e.g. titanium dioxide), combined in any ratios necessary to achieve the targeted electromagnetic radiation protection spectrum.

In other embodiments, the composition further comprises a skin absorption promoting agent. The absorption promoting agents are substances capable of improving the diffusion of active ingredients in the epidermis, in particular across the inherent barrier function of the stratum corneum. These adjuvants can be classified in different families according to their chemical structure. Suitable skin absorption promoting agents are known in the art. As an example of absorption promoting agents, dioxolane derivatives such as isopropylidene glycerol, marketed under the name Solketal or 2n-nonyl 1-3 dioxolane; or diethylene glycol monoethyl ether (for example that marketed under the Tradename Transcutol®) can in particular be mentioned. In addition, micro or mini hyaluronic acid (HA), i.e. low molecular weight HA below 10,000 DA, can be used to enhance skin penetration. Absorption promoting agents are also described in the following chemical families: polyols, fatty acids, esters of fatty acids alcohols and amides. As an example of substances representative of these families, propylene glycol monocaprylate or Capryol 90, caprylic acid, diisopropyl adipate, polysorbate 80, 2-octyl dodecanol and 1-dodecylazacyclohepta-2-one or Azone, can in particular be mentioned. Substances presenting properties of absorption promoting agents can also be found in the family of sulphoxides (such as for example dimethylsulphoxide), terpenes (for example d-limonene), alkanes (for example N-heptane) or organic acids (for example alpha hydroxy acids such as glycolic acid and lactic acid, and salts thereof, or salicylic acid and salicylates). The quantity of absorption promoting agent in the compositions according to the invention, can, in aspects, vary from, e.g., about 0.01% to about 12% by weight of the total composition.

The cosmetic, pharmaceutical, and dermatological compositions of the invention optionally further comprise one or more cosmetic auxiliaries, as are used conventionally in such compositions, for example preservatives, bactericides, perfumes, substances for preventing foaming, dyestuffs, pigments which have a coloring effect, thickening agents, surfactant substances, emulsifiers, softening, moisturizing and/or moisture-retaining substances, exfoliating agents, fats, oils, waxes or other conventional constituents of a cosmetic or dermatological formulation, such as alcohols, polyols, polymers, foam stabilizers, electrolytes, organic solvents or silicone derivatives.

The cosmetic, pharmaceutical, or dermatological compositions of the invention can be conventionally prepared and then used to provide treatment, care, and cleansing of the skin, and as a make-up product in decorative cosmetics, for example, as dry powder formulations of minerals, natural minerals and earth-derived pigments. For administration, the endocannabinoid mimetic blend of the invention can be topically applied to the skin in cosmetic and dermatological compositions of the invention in the manner conventional for cosmetics.

Cosmetic, pharmaceutical, and dermatological compositions of the invention can exist in various forms. For example, the compositions of the invention can be in the form of a cream, a solution, a serum, an anhydrous preparation, an emulsion or microemulsion of the type water-in-oil (W/O) or of the type oil-in-water (O/W), a multiple emulsion, for example of the type water-in-oil-in-water (W/O/W), a gel, a solid stick, an ointment, a dermal patch, a transdermal patch, or an aerosol. It is also advantageous to administer an endocannabinoid mimetic blend of the invention in encapsulated form, for example in collagen matrices and other conventional encapsulation materials, for example as cellulose encapsulations, in gelatin, in wax matrices or as liposomal encapsulations. It is may also possible and advantageous within the scope of the present invention to add an endocannabinoid mimetic blend of the invention to aqueous systems or surfactant compositions for cleansing the skin or scalp.

The use of a endocannabinoid mimetic containing blend of the invention can be combined with known anti-aging, wound healing, and/or OTC monograph topical technologies in cosmetic or therapeutic drug dermatological compositions, which may include vitamin A and/or its derivatives (for example, all-E-retinoic acid, 9-Z-retionoic acid, 13-Z-retinoic acid, retinal, retinyl esters, e.g. retinyl palmitate and retinoate esters, e.g. ethyl lactyl retinoate), alpha hydroxy acid, beta hydroxy acid, antioxidants, peptides, growth factors, stem cells, and OTC approved monograph ingredients for acne, dandruff, external analgesics, topical protectants, anti-microbials, topical OTC and prescription compounds (e.g. salicylic acid, hydroquinone, corticosteroids, and growth factors) individually or in combination, is thus likewise within the scope of the present invention. The anti-inflammatory, pain relief, anti-aging, acne, wound healing, depigmentation, or other effects of the present invention may be synergistic in nature in such combinations.

As referred to herein, gene expression can be measured in a cell that has been exposed to at least one compound and/or natural extract. Suitable cell types for measuring effects of compositions of the invention can include fibroblasts, keratinocytes, mast cells, melanocytes, Langerhans cells, and cells of sweat or oil glands. When expression of a gene is increased or decreased, the increase or decrease is determined by comparison of expression of the same gene(s) of interest in cells which have not been exposed to the at least one compound and/or natural extract. In some cases, comparisons can be made to equivalent concentrations of the cannabinoid cannabidiol. Methods of analyzing gene expression are well known in the art, and include, but are not limited to, polymerase chain reaction (PCR; e.g., reverse transcriptase polymerase chain reaction (RT-PCR), competitive RT-PCR, Real-time RT-PCR, etc.), hybridization methods (Northern blotting, Microarray, etc.), Taq-based techniques (SAGE, RNA-seq, etc.), and DNA chips.

The following examples are exemplary of the present invention and should not be construed as in any way limiting its scope.

### Example 1

Cell cultures: A human skin fibroblast cell culture (or cultures) was obtained through the Coriell Cell Repository from the National Institute on Aging Cell Repository (Camden, N.J.), or Promocell GmbH (Heidelberg, Germany). The initial culture was selected from the following cell lines: AG13066, AG11557, AG11796 or GM03651E. The cell lines were derived from donors as follows:

AG13066: 42-year-old human female
AG11557: 36-year-old human male
AG11796: 35-year-old human female
GM03651E: 25-year-old human female

Culture media: Cells were grown in ready to use Fibroblast Growth Medium 2 from Promocell GmbH (Heidelberg, Germany) containing basal media supplemented with 0.02 ml/ml fetal calf serum, 1 ng/ml recombinant human basic fibroblast growth factor, and 5 μg/ml of recombinant human insulin. During the 24-hour experimental phase, cells were maintained in only the basal medium which has the test compound(s) added. All cultures were incubated at 37° C. with 5% CO₂ in a humidified chamber.

Cell culture growth and expansion phase: The selected cell culture line vial(s) were taken from storage in liquid nitrogen, thawed in a 37 C° water bath and pipetted into 20 ml of fibroblast growth medium in a sterile 50 ml centrifuge tube. The cells were gently mixed via pipetting and seeded into sterile 75 cm² culture flask(s). The flask(s) were placed in the previously described incubator at 37° C. with 5% CO₂. After 24 hrs, the cells were examined using an inverted light microscope for attachment to the flask(s) and overall viability. If the cells show good attachment, the media were aspirated under sterile conditions in a class II laminar flow hood, replaced with a fresh 12 ml of growth media and returned to the incubator. The culture flask(s) were examined daily for several factors: viability, level of confluence (coverage of the surface of the flask with cells) and possible contamination. Media were replaced as needed every 2-3 days until the flask(s) reach 90-100% confluence (almost complete coverage of the flask surface). When the cells reached that level of confluence, the cultures were expanded.

Expansion of cell culture, once the confluence threshold was achieved, first required the use of the Promocell Cell Detach Kit to release the cell adhesion to the culture flask. This was done using the manufacturer's protocol, summarized as follows:
Prepare the reagents by equilibrating at room temperature or in a water bath.
Aspirate the growth media and rinse the cells with room temperature phosphate buffered saline. Carefully aspirate the PBS from the culture vessel and add 7.5 ml of HEPES BSS. Gently agitate for 15 seconds.
Remove the HEPES BSS and add 7.5 ml of Trypsin/EDTA Solution. Close the flask and examine the cells under a microscope for detachment. Once the cells begin to detach, gently tap the flask to loosen all remaining cells.
Add 7.5 ml of Trypsin Neutralizing Solution and gently agitate. Carefully pipette the created cell suspension into a sterile 50 ml centrifuge tube and place in the centrifuge for 3-5 minutes at 220 g to form a cell pellet.
Remove from the centrifuge and aspirate the solution to leave only the cell pellet.
The cells will be expanded at 3:1 so 36 ml of fibroblast growth media should be added and gently mixed to put the cells back into suspension.
Prepare 3 new sterile culture flasks, noting the cell line and passage number and pipette an equal (12 ml) amount of the cell suspension into each and return to the incubator.

Experimental phase: When the appropriate quantity of cells had been grown, they were detached as described above, but at the final step were seeded into each well of one or more 6 well dishes. Each well received 2 ml of the cell suspension so they were seeded equally. The dishes were returned to the incubator until 85-95% confluency was reached.

It is at this stage that all wells were rinsed in PBS after aspirating the growth media. The compound(s) to be tested were previously mixed into a suitable solvent (e.g., DMSO or ethanol) that can be diluted to the desired concentration of test compound (10 μl) in the culture wells without exceeding maximum solvent concentrations and adversely effecting cell viability, and mixed with basal media. NOTE: Standard maximum levels of solvent are 0.1% for DMSO and 0.5% for ETOH. The concentration of each test compound is found in Tables 1 & 2.

Every 2 wells on the 6 well plate served as a biological replicate for the purposes of RNA isolation and genetic expression evaluations making each 6 well dish an n=3 for the test compound(s) contained in the media. Negative control plates (no test compound(s) and only basal media) were also generated. The plates were returned to the incubator for 24 hrs.

At the end of the 24 hr time period, the plates were removed from the incubator, the test media aspirated, and the cells rinsed with PBS. RNA isolation is performed using the BioRad Aurum Total RNA Mini Kit (Hercules, Calif.) per manufacturer's protocol; which is described, in brief, below:
Add 350 μl of lysis buffer to each well. Pipet multiple times to ensure thorough lysis. Add lysed cells to the collection tube.
Add 350 μl of 70% ETOH to the collection tube and mix thoroughly.
Pipet the generated lysate onto the spin column placed in a new 2 ml tube. Centrifuge for 30 sec between 8,000 and 10,000 g.
Place the spin column containing the now bound RNA, in a new 2 ml tube. Add 700 μl of low stringency wash solution to the spin column. Centrifuge for 30 sec between 8,000 and 10,000 g. Discard the flow through and replace spin column.
Add 80 μl of DNase I dilution to the spin column and incubate at room temperature for 15 minutes.
Add 700 μl of high stringency wash solution to the column and centrifuge for 30 sec between 8,000 and 10,000 g. Discard flow through.
Add 700 μl of low stringency wash solution to the column and centrifuge for 60 sec between 8,000 and 10,000 g. Discard flow through.
Centrifuge for 2 minutes to remove residual wash solution.
Transfer spin column to new 1.5 ml collection tube and add 80 μl of elution solution to the column. Allow 1 minute for saturation of the membrane and then centrifuge for 2 minutes to complete the RNA elution.
Quantify the quantity of RNA and store at −20 C° until use (no more than 1 month)

RNA is quantified by Optical Density readings at 260 and 280 nm using a DeNovix DS11+ spectrophotmeter. A 260/280 ratio of ˜2.0 is generally accepted as “pure” for RNA and is used to determine if a sample is of sufficient quality to be used to generate viable gene expression data.

List of test compounds: The compounds and compositions tested are indicated in Tables 1 and 2 including β caryophyllene, N-alkylamides, honokiol, magnolol, curcumin, eugenol, ginkolide, triptolide, N-palmitoylethanolamide, triterpene alcohols & triterpendiol monoesters (faradiol), 7-hydroxyflavone, 3,7-dihydroxyflavone, N-acetyl L-cysteine, ginsenosides, disophenol, isomenthone, menthone, limonene, docosahexaenoic acid (DHA), eicosapentaenoic acid (EPA), myristicin, allicin, N-oleoylethanolamide, menthol, camphor, and methyl salicylate and for certain compositions thereof. Concentrations of test compounds are indicated in Tables 1 and 2.

The genes tested were:
Cannabinoid receptor 1 (CB1(CNR1))
Cannabinoid receptor 2 (CB2 (CNR2))
Fatty acid amide hydrolase (FAAH)
Monoacylglycerol lipase (MGLL, i.e., MAGL)
Peroxisome proliferator-activated receptor gamma (PPARG)
Nuclear factor kappa-light-chain-enhancer of activated B cells (NFκβ)
Interleukin 1 Alpha (IL1alpha)
Cyclooxygenase 1 (PTGS1, i.e., COX1)
Transient receptor potential cation channel subfamily A member 1 (TRPA1)
Transient receptor potential cation channel subfamily V member 1 (TRPV1)
Transient receptor potential cation channel subfamily M member 8 (TRPM8)
Hemoglobin Subunit Beta (HBB) REFERENCE GENE
Ribosomal Protein L13a (RPL13A) REFERENCE GENE

The gene descriptions are:
CB1 (CNR1)—Cannabinoid receptor type 1 (CB1), also known as cannabinoid receptor 1, is a G protein-coupled cannabinoid receptor that in humans is encoded by the CNR1 gene. The human CB1 receptor is expressed in the peripheral nervous system and central nervous system. It is activated by: endocannabinoids, a group of retrograde neurotransmitters that include anandamide and 2-arachidonoylglycerol (2-AG); plant phytocannabinoids, such as the compound THC which is an active ingredient of the psychoactive drug cannabis. The primary endogenous agonist of the human CB1 receptor is anandamide.
CB2 (CNR2)—The cannabinoid receptor type 2 (CB2), is a G protein-coupled receptor from the cannabinoid receptor family that in humans is encoded by the CNR2 gene. It is closely related to the cannabinoid receptor type 1. The principal endogenous ligand for the CB2 receptor is 2-Arachidonoylglycerol (2-AG). The discovery of this receptor helped provide a molecular explanation for the established effects of cannabinoids on the immune system.
FAAH—Fatty acid amide hydrolase or FAAH (anandamide amidohydrolase) is a member of the serine hydrolase family of enzymes. In humans, it is encoded by the gene FAAH, primarily responsible for the degradation of anandamide to arachidonic acid.
MAGL—Monoacylglycerol lipase, is an enzyme that, in humans, is encoded by the MGLL gene. MAGL is a member of the serine hydrolase superfamily and functions to degrade 2-arachidonoylglycerol (2-AG).
PPARg—Peroxisome proliferator-activated receptor gamma (PPAR-γ or PPARG), is a type II nuclear receptor (protein regulating genes) that in humans is encoded by the PPARG gene. a member of the nuclear receptor family of ligand-activated transcription factors, which heterodimerise with the retinoic X receptor (RXR) to regulate gene expression. PPARG binds peroxisome proliferators such as fatty acids and controls the peroxisomal beta-oxidation pathway of fatty acids. It is a key regulator of adipocyte differentiation and glucose homeostasis.
NFKB—NF-κB (nuclear factor kappa-light-chain-enhancer of activated B cells) is a protein complex that controls transcription of DNA, cytokine production and cell survival. NF-κB is found in almost all animal cell types and is involved in cellular responses to stimuli such as stress, cytokines, free radicals, heavy metals, ultraviolet irradiation, oxidized LDL, and bacterial or viral antigens. NF-κB plays a key role in regulating the immune response to infection.
IL1a—Interleukin 1 alpha (IL-1α) also known as hematopoietin 1 is a cytokine of the interleukin 1 family that in humans is encoded by the ILIA gene. In general, Interleukin 1 is responsible for the production of inflammation, as well as the promotion of fever and sepsis. IL-1α is produced mainly by activated macrophages, as well as neutrophils, epithelial cells, and endothelial cells. It possesses metabolic, physiological, haematopoietic activities, and plays one of the central roles in the regulation of the immune responses. It binds to the interleukin-1 receptor. It is on the pathway that activates tumor necrosis factor-alpha.
COX (PTGS1)—Cyclooxygenase (COX), officially known as prostaglandin-endoperoxide synthase (PTGS), is an enzyme that is responsible for formation of prostanoids, including thromboxane and prostaglandins such as prostacyclin, from arachidonic acid. A member of the animal-type heme peroxidase family, it is also known as prostaglandin G/H synthase. The specific reaction catalyzed is the conversion from arachidonic acid to Prostaglandin H2, via a short-living Prostaglandin G2 intermediate. Inhibition of COX can provide relief from the symptoms of inflammation and pain. The two isozymes found in humans, PTGS1 and PTGS2, are frequently called COX-1 and COX-2 in medical literature.
TRP GENES: TRP channels are a large group of transient receptor potential ion channels consisting of six protein families, located mostly on the plasma membrane of numerous human and animal cell types, and in some fungi. TRP channels in vertebrates are ubiquitously expressed in many cell types and tissues. There are about 28 TRP channels that share some structural similarity to each other. These are grouped into two broad groups: group 1 includes TRPC (“C” for canonical), TRPV (“V” for vanilloid), TRPM (“M” for melastatin), TRPN and TRPA. In group 2 there are TRPP (“P” for polycystic) and TRPML (“ML” for mucolipin).
TRPA1—The TRPA family is made up of 7 subfamilies, the TRPA1s have been the most extensively studied subfamily; and are believed to function as mechanical stress, temperature, and chemical sensors. TRPA1 is known to be activated by compounds such as isothiocyanate (which are the pungent chemicals in substances such as mustard oil and wasabi) and Michael acceptors (e.g. cinnamaldehyde). These compounds are capable of forming covalent chemical bonds with the protein's cysteins. Non-covalent activators of TRPA1 also exists, such as methyl salicylate, and menthol.
TRPV1—TRPV (vanilloid) also has 6 members: TRPV1: (HEAT) capsaicin, eugenol, gingerol, cannabinoids, endocannabinoids, lidocaine; inflammatory and neuropathic pain. TRPV2: CBD, probenecid; inflammatory pain. TRPV3: camphor, carvacol, thymol and AA, PUFA resolvins: inflammatory and nociceptor. TRPV4: UVB irradiation>inflammation from TRPV4 activation in keratinocytes.
TRPM8—Functional TRPM channels are believed to form tetramers. The TRPM family consists of eight different channels, TRPM1-TRPM8. TRPM are activated by steroids, types include TRPM2 (inflammatory pain), TRPM3 (neurogenic pain) TRPM8: (COLD).

Performance of custom microarray: The gene expression data was generated by utilizing the isolated RNA samples in custom designed cDNA microarrays in a 96 well format. The arrays were set up to test duplicates of 11 genes of interest with 2 housekeeping/reference genes for 3 biological replicates on each plate.

The remaining wells served as assay controls for genomic DNA contamination, polymerase reaction efficiency and transcription rates.

The arrays were performed by using equal amounts of sample RNA from each of the tested compounds to be amplified using the BioRad iScript cDNA synthesis kit (Hercules, Calif.) per manufacturer's instructions. Briefly this consisted of taking the designated amount of starting RNA and mixing it with the required amount of synthesis buffer/reverse transcriptase and performing a series of amplification reactions (5 min 25° C. priming; 20 min 46° C. reverse transcription and 1 min 95° C. reverse transcription inactivation) to generate the cDNA needed for the array.

This cDNA template was mixed with enough BioRad SsoAdvanced Universal SYBR Supermix (Hercules, Calif.) to generate enough sample for the 96 well plate (20 μl per well). The cDNA served as the template to the specific gene primers in each of the wells which undergo polymerase chain reaction (PCR) to amplify the gene marker contained in that well. A typical PCR reaction consists of Denaturing, and Annealing/Extension steps repeated for approximately 40 cycles. As these genes are amplified, the SYBR mix gives of a fluorescence which is detected by the BioRad iCycler CFX Touch (Hercules, Calif.) system in real time. This fluorescence eventually breaks a basal level known as the background level. The cycle at which these levels are broken relative to the reference genes and the levels of an untreated control sample determine the fold increase or decrease of the gene expression seen in cells treated by the tested compounds.

Custom microarray analysis: Completed arrays were analyzed using the BioRad CFX Manager software. During the analysis four objectives were examined:

Objective 1—Compare the gene expression data from an untreated sample with any/all of the tested compound treated samples to determine fold change and p-values for every gene measured by the microarray.

Objective 2—Identify differentially expressed genes for the comparison generated in Objective 1 using standard criteria (specifically, an absolute fold change value >1.5, a log ratio p-value <0.05).

Objective 3—Identify test compounds that have the greatest fold changes, the largest number of differentially expressed genes, or a combination of both that indicates a beneficial profile for pain, inflammation and/or skin function. These compounds will inform the initial formulations for additional testing.

Objective 4—Identify comparative efficacy of gene response for all compounds and compositions tested vs. equivalent concentrations of CBD and approved external analgesic therapeutic compounds (methyl salicylate, menthol, camphor).

Results: see Tables 1 and 2 for individual ingredients and compositions test results; composition ingredient key follows:

Composition 1: B-caryophyllene, curcumin, N-palmitoylethanolamide, docosahexaenoic acid (DHA) eicosapentaenoic acid (EPA), triptolide
Composition 2: B-caryophyllene, curcumin, N-palmitoylethanolamide, diosphenol, docosahexaenoic acid (DHA), eicosapentaenoic acid (EPA), triptolide
Composition 3: B-caryophyllene, curcumin, N-palmitoylethanolamide, N-oleoylethanolamide, 7-hydroxyflavone, docosahexaenoic acid (DHA) eicosapentaenoic acid (EPA), triptolide
Composition 4: B-caryophyllene, curcumin, N-palmitoylethanolamide, N-oleoylethanolamide, N-alkylamides, 7-hydroxyflavone, docosahexaenoic acid (DHA) eicosapentaenoic acid (EPA), triptolide, ginsenoside
Composition 5: B-caryophyllene, curcumin, N-palmitoylethanolamide, N-oleoylethanolamide, N-alkylamides, 7-hydroxyflavone, diosphenol, docosahexaenoic acid (DHA) eicosapentaenoic acid (EPA)

TABLE 1
(Desired Gene Direction, i.e., ↑ = upregulation, ↓ = downregulation/Gene)

| | CNR1↑ | CNR2↑ | FAAH↓ | MAGL↓ | IL1A↓ | NFKB1↓ | PPARG↑ | PTGS1↓ | TRPA1↑ | TRPM8↑ | TRPV1↑ |
|---|---|---|---|---|---|---|---|---|---|---|---|
| 10 uM CBD | 1.5 | 1.7 | 2.2 | 1.8 | 20.5 | 2.3 | 8.1 | 4.2 | 2.7 | 6.1 | −2 |
| Fibroblasts | | | | | | | | | | | |
| 10 uM CBD | 2.6 | 0 | −3.1 | −2.3 | −1.8 | −1.3 | −4.7 | 2.3 | −1.7 | 2.5 | 2.2 |
| Keratinocytes | | | | | | | | | | | |
| Composition 2 | 82.1 | 57.3 | 1.1 | −12.1 | 0 | 1.1 | −1.5 | 0 | 0 | 66.5 | 1.2 |
| Composition 3 | 2.7 | 2.8 | −1.6 | −1.1 | 2.2 | −3.6 | −3.1 | −3.1 | 2 | 3.6 | −5.6 |
| Composition 5 | 3.4 | 1.2 | 2.5 | 2.9 | 0 | −1.5 | −2.3 | −3.1 | 0 | 1.3 | −5.5 |
| Composition 1 | 0 | 0 | −1.9 | 5.2 | 0 | −21.1 | −4.1 | −4.6 | 1.4 | 0 | −8.1 |
| Composition 4 | 0 | 0 | 1.5 | 7.9 | 0 | −2 | −1.6 | −1.9 | 0 | 0 | 0 |

DIRECT ECS

| | | | | | | | | | | | |
|---|---|---|---|---|---|---|---|---|---|---|---|
| 10 uM Curcumin | 47.4 | 55.4 | 1.8 | −5 | 4.9 | 1.1 | −1.4 | 0 | 4.9 | 47.9 | 0 |
| 10 uM Eugenol | 147.7 | 108.8 | 3.0 | −9.8 | 39.2 | 2.5 | 1.6 | −3.1 | 30.1 | 152.2 | 0 |

INDIRECT ECS

| | | | | | | | | | | | |
|---|---|---|---|---|---|---|---|---|---|---|---|
| 10 uM N- | 5.9 | 3.9 | −1.1 | 2.8 | −1.2 | 1.5 | 2.6 | 1.1 | 8.2 | 5.1 | −3.2 |
| palmitoylethanolamide | | | | | | | | | | | |
| 10 uM N- | −1.9 | −2.4 | −1.1 | 2.5 | −1.9 | 1.5 | 2.4 | 1.5 | 1.1 | −1.4 | −1.6 |
| Oleylethanolamide | | | | | | | | | | | |
| 10 uM Ginsenoside | 0 | 0 | −1.9 | −1.3 | −3.2 | 1.8 | −1.6 | 7.9 | 1.5 | −5.1 | −2.3 |

ECS INFLAMMATORY NUCLEAR

| | | | | | | | | | | | |
|---|---|---|---|---|---|---|---|---|---|---|---|
| 10 uM Bucha extract | 0 | 0 | 83.8 | 27.1 | 0 | 49.4 | 29.8 | 0 | 0 | 0 | 0 |
| (Diosphenol) | | | | | | | | | | | |
| 10 uM Honokiol | 1.2 | 1.3 | 2.3 | 2.7 | 1.8 | 1.6 | 2.3 | 1.5 | 1.4 | 1.8 | −3.4 |

ECS INFLAMMATORY ENZYMATIC

| | | | | | | | | | | | |
|---|---|---|---|---|---|---|---|---|---|---|---|
| 10 uM B- | 0 | 0 | −1.6 | 1 | 0 | −1.3 | −1.6 | −1.5 | 0 | 5.5 | 2 |
| Caryphyllene | | | | | | | | | | | |
| 10 uM Ginkolide | 1.1 | −1.2 | −1.1 | 2.3 | −3.3 | −1.2 | −1.3 | −1.3 | 1.1 | 2.1 | −2.6 |

ECS INFLAMMATORY CYTOKINE

| | | | | | | | | | | | |
|---|---|---|---|---|---|---|---|---|---|---|---|
| 10 uM Triptolide | 0 | 10.1 | −3.2 | −2.1 | 15.5 | −2 | 1.1 | 0 | 26.1 | 47.6 | 0 |
| 10 uM 7- | −2.3 | −1.1 | 1.2 | 2 | −2.8 | 1.1 | 1.4 | 1.4 | −1.2 | −1.7 | −1 |
| Hydroxyflavone | | | | | | | | | | | |
| 10 uM N-Acetyl | 0 | 0 | −1.6 | −2.4 | 0 | −4 | −3.7 | 0 | 0 | 0 | 0 |
| Cysteine | | | | | | | | | | | |

ECS TRP PATHWAY

| | | | | | | | | | | | |
|---|---|---|---|---|---|---|---|---|---|---|---|
| 10 ppm Echinacea | 0 | −2.3 | −1.4 | −1.6 | 1.7 | 2.9 | −1 | 9.2 | 2.7 | −2.5 | −1.3 |
| purpurea extract | | | | | | | | | | | |
| 10 PPM Algal Oil | 0 | 0 | 2 | 3 | −1.2 | 1.7 | 1.7 | 1.6 | 4.3 | 1.1 | −1.2 |
| (DHA/EPA) | | | | | | | | | | | |
| 10 uM Methyl | 0 | −1.3 | −1.2 | 2.4 | 0 | 1.9 | 1.8 | 1.4 | 3 | −1.4 | −1.7 |
| Salicylate | | | | | | | | | | | |
| 10 uM Camphor | 0 | −2.5 | −3.3 | −2 | −1.6 | −1.5 | −2.5 | 3 | −1.6 | −6.7 | −3.9 |
| 10 uM Menthol | 0 | −3.1 | 2 | 2.7 | −1.2 | 3.4 | 1.8 | 7.8 | 2.5 | −1.6 | −1.5 |

TABLE 2

| | | | | |
|---|---|---|---|---|
| COMP 1 | COMP 2 | COMP 3 | COMP 4 | COMP 5 |

DIRECT ECS

| | | | | | |
|---|---|---|---|---|---|
| 10 uM Curcumin | x | x | x | x | x |
| 10 uM Eugenol | | | | | |

INDIRECT ECS

| | | | | | |
|---|---|---|---|---|---|
| 10 uM N-palmitoylethanolamide (PEA) | x | x | x | x | x |
| 10 uM N-Oleylethanolamide (OEA) | | | x | x | x |
| 10 uM Ginsenoside | | | | x | |

ECS INFLAMMATORY NUCLEAR

| | | |
|---|---|---|
| 10 uM Bucha extract (Diosphenol) | x | x |
| 10 uM Honokiol | | |

ECS INFLAMMATORY ENZYMATIC

| | | | | | |
|---|---|---|---|---|---|
| 10 uM B-Caryophyllene | x | x | x | x | x |
| 10 uM Ginkolide | | | | | |

ECS INFLAMMATORY CYTOKINE

| | | | | | |
|---|---|---|---|---|---|
| 10 uM Triptolide | x | x | x | x | |
| 10 uM 7-Hydroxyflavone | | | x | x | x |
| 10 uM N-Acetyl Cysteine | | | | | |

ECS TRP PATHWAY

| | | | | | |
|---|---|---|---|---|---|
| 10 ppm Echinacea purpurea extract | | | | x | x |
| 10 PPM Algal Oil (DHA/EPA) | x | x | x | x | x |
| 10 uM Methyl Salicylate | | | | | |
| 10 uM Camphor | | | | | |
| 10 uM Menthol | | | | | |

Table 1 depicts fold increases or decreases in gene expression of each listed gene following treatment of cells with the listed compound, natural extract, or composition (i.e. a combination of compounds, natural extracts, or a combination thereof) compared to gene expression of the same gene in untreated cells. Table 2 indicates the compounds and natural extracts, shown by an “x,” contained within each tested composition. The concentration of the compound or natural extract as tested is indicated on the Tables 1 and 2 as either uM or ppm. In the case of a tested composition, the concentration of each compound or natural extract within the composition is equivalent to its standalone test concentration. In other words, if compound X was tested at 10 uM individually, then compound X is present as a component of a tested composition at 10 uM concentration.

For a combination of at least one direct endocannabinoid compound, a second indirect endocannabinoid compound, a third ECS related pathway anti-inflammatory compound consisting of one or more compounds targeting specific anti-inflammatory pathways, e.g. nuclear, enzymatic, cytokine, and a fourth ECS related TRP pathway compound targeting any one of the TRP ionotropic pathways, and a fifth compound that affects a pathway not affected by any of the preceding compounds are identified that indicate a beneficial profile for pain, itch, inflammation, and/or ECS homeostasis support (cellular proliferation, differentiation, autophagy, apoptosis, senescence, lipid synthesis, barrier repair, and microbiome support). Certain combinations demonstrated (a) a synergistic gene expression effect in comparison to the gene expression elicited by the compounds of the combination(s) individually (see, e.g., bolded entries in Table 1), and/or (b) a greater beneficial (increase or decrease) modulation in gene expression in comparison to an equivalent concentration of cannabidiol, and/or (c) a greater increase in gene expression for at least one the TRP gene selected from the group consisting of TRPV1, TRPA1, TRPM8.

Specifically for Composition 2 outlined hereunder, the combination of compounds selected from the groups consisting of direct and indirect endocannabinoid compounds, anti-inflammatory compounds from each of the three ECS related anti-inflammatory pathways (nuclear, enzymatic, and cytokine) and the ECS related TRP pathway for pain, itch, cellular proliferation, differentiation, autophagy, apoptosis, senescence, lipid synthesis and barrier function compounds, includes B-caryophyllene, curcumin, N-palmitoylethanolamide, diosphenol, isomenthone, menthone, limonene, docosahexaenoic acid (DHA), eicosapentaenoic acid (EPA), and triptolide produced synergistic beneficial gene test results for CB1 and MAGL. The group chemical class designation and compound identification for the composition is:
Direct ECS pathway (CB1, CB2): Curcuminoids: curcumin
Indirect ECS pathway (FAAH, MAGL): Fatty Acid Amides: N-palmitoylethanolamide
ECS related Anti-inflammatory nuclear pathway (PPARg): Monoterpenes: diosphenol, isomenthone, menthone, limonene
ECS related Anti-inflammatory enzymatic pathway (PTGS1): Sesquiterpenes: B-caryophyllene
ECS related Anti-inflammatory cytokine pathway: (ILIA, NFKB): Diterpenes: Triptolide
ECS related TRP pathway: (TRPV1, TRPA1, TRPM8): PUFAs: docosahexaenoic acid (DHA), eicosapentaenoic acid (EPA)

Summarily the findings for composition 2 were:
Gene expression response superior to equivalent concentration of cannabidiol for CB1, CB2, FAAH, MAGL, IL-1A, NFKB, TRPM8
Synergistically superior to individual composition compounds in gene response for CB1 & MAGL
54-fold more effective agonist for CB1 than CBD; 33-fold more effective agonist for CB2
14-fold more effective antagonist for MAGL suppression than CBD
Superior gene no coreceptor response for TRPM8 compared to equivalent concentrations of cannabidiol
Superior gene nocioreceptor response for TRPM8 compared to menthol, methyl salicylate & camphor

Specifically for Composition 3 outlined hereunder, the combination of compounds selected from the groups consisting of direct and indirect endocannabinoid compounds, anti-inflammatory compounds from each of the three ECS related anti-inflammatory pathways (nuclear, enzymatic, and cytokine) and the ECS related TRP pathway for pain, itch, cellular proliferation, differentiation, autophagy, apoptosis, senescence, lipid synthesis and barrier function compounds, includes B-caryophyllene, curcumin, N-palmitoylethanolamide, N-oleoylethanolamide, 7-hydroxyflavone, docosahexaenoic acid (DHA), eicosapentaenoic acid (EPA), and triptolide produced synergistic beneficial gene test results for NFKB, PTGS1 and TRPV1. The group chemical class designation and compound identification for the composition is:
Direct ECS pathway (CB1, CB2): Curcuminoids: curcumin
Indirect ECS pathway (FAAH, MAGL): Fatty Acid Amides: N-oleoylethanolamide, N-palmitoylethanolamide
ECS related Anti-inflammatory nuclear pathway (PPARg): Monoterpenes: diosphenol, isomenthone, menthone, limonene
ECS related Anti-inflammatory enzymatic pathway (PTGS1): Sesquiterpenes: B-caryophyllene
ECS related Anti-inflammatory cytokine pathway: (ILIA, NFKB): Diterpenes: Triptolide; Hydroxyflavones: 7-hydroxyfalvone
ECS related TRP pathway: (TRPV1, TRPA1, TRPM8): PUFAs: docosahexaenoic acid (DHA), eicosapentaenoic acid (EPA)
   Gene expression response superior to equivalent concentration of cannabidiol for CB1, CB2, FAAH, MAGL, IL-1A, NFKB, PTGS1, TRPV1
   Synergistically superior to individual composition compounds in gene response for NFKB, PTGS1, and TRPV1

### Example 2

Cell cultures: A human skin keratinocyte cell culture (or cultures) is obtained through the ThermoFisher Scientific (Waltham, Mass.) or Promocell GmbH (Heidelberg, Germany). The initial culture was selected from one of the following, C055C, 3C0647, or

C12003. The cell lines were derived from donors as follows:
C055C: Single donor adult
3C0647: Single donor adult, lightly pigmented
C12003: Single donor adult

Culture media: Cells were grown in ready to use Keratinocyte Growth Medium 2 from Promocell GmbH (Heidelberg, Germany) containing basal media supplemented with Bovine Pituitary Extract 0.004 ml/ml, Epidermal Growth Factor (recombinant human) 0.125 ng/ml, Insulin (recombinant human) 5 μg/ml, Hydrocortisone 0.33 μg/ml, Epinephrine 0.39 μg/ml, Transferrin (recombinant human) 10 μg/ml, and CaCl2 0.06 mM. During the 24-hour experimental phase, cells were maintained in only the basal medium which has the test compound(s) added. All cultures were incubated at 37° C. with 5% CO₂ in a humidified chamber.

Cell culture growth and expansion phase: The cell culture growth and expansion phase was carried out in accordance with the protocol set forth in the cell culture growth and expansion phase section of Example 1.

Experimental phase: The experimental phase was carried out in accordance with the protocol set forth in the experimental phase section of Example 1.

List of test compounds: cannabidiol

The genes tested: Tested genes were the same as tested in Example 1.

The gene descriptions: See Example 1.

Performance of custom microarray: Performance of custom microarray was carried out in accordance with the protocol set forth in the performance of custom microarray section of Example 1.

Custom microarray analysis: Analysis was carried out in accordance with the protocol set forth in the custom microarray analysis section of Example 1.

Results: The results for cannabidiol are shown in Table 1.

### Example 3

Cell cultures: Received and began culture of pooled keratinocyte line from Promocell (C-12006; Lot #448Z026). Cells were seeded into a 75 cm2 flask and 2 6 well dishes for expansion and exposure per manufacturer's protocol using Keratinocyte Growth Medium (C2011B), Keratinocyte Supplement Mix (C39016) and CaCl (C34005). 2 ml of prepared solution were added to each of the 6 wells and 7 ml was added to the 75 cm2 flask.

Culture media: Cells were grown in ready to use Keratinocyte Growth Medium 2 from Promocell GmbH (Heidelberg, Germany) containing basal media supplemented with Bovine Pituitary Extract 0.004 ml/ml, Epidermal Growth Factor (recombinant human) 0.125 ng/ml, Insulin (recombinant human) 5 μg/ml, Hydrocortisone 0.33 μg/ml, Epinephrine 0.39 μg/ml, Transferrin (recombinant human) 10 μg/ml, and CaCl2 0.06 mM. During the 24-hour experimental phase, cells were maintained in only the basal medium which has the test compound(s) added. All cultures were incubated at 37° C. with 5% CO₂ in a humidified chamber.

Cell culture growth and expansion phase: The selected cell culture (C-12006) were taken directly from the shipping container from Promocell, thawed in a 37 C° water bath and pipetted into 20 ml of prepared Keratinocyte Growth Medium (C2011B) containing {Keratinocyte Supplement Mix (C39016) and CaCl (C34005) per manufacturer protocol} in a sterile 50 ml centrifuge tube. The cells were gently mixed via pipetting and seeded into sterile 75 cm² culture flask(s). The flask(s) were placed in the previously described incubator at 37 C° with 5% CO₂. After 24 hrs, the cells were examined using an inverted light microscope for attachment to the flask(s) and overall viability. If the cells showed good attachment, the media was aspirated under sterile conditions in a class II laminar flow hood, replaced with a fresh 12 ml of keratinocyte growth media and returned to the incubator. The culture flask(s) were examined daily for several factors: viability, level of confluence (coverage of the surface of the flask with cells) and possible contamination. Media was replaced as needed every 2-3 days until the flask(s) reach 90-100% confluence (almost complete coverage of the flask surface). When the cells reached that level of confluence, the cultures are expanded.

Expansion of cell culture, once the confluence threshold was achieved, first required the use of the Promocell Cell Detach Kit to release the cell adhesion to the culture flask. This was done using the manufacturer's protocol, summarized as follows:
Prepare the reagents by equilibrating at room temperature or in a water bath.
Aspirate the growth media and rinse the cells with room temperature phosphate buffered saline. Carefully aspirate the PBS from the culture vessel and add 7.5 ml of HEPES BSS. Gently agitate for 15 seconds.
Remove the HEPES BSS and add 7.5 ml of Trypsin/EDTA Solution. Close the flask and examine the cells under a microscope for detachment. Once the cells begin to detach, gently tap the flask to loosen all remaining cells.
Add 7.5 ml of Trypsin Neutralizing Solution and gently agitate. Carefully pipette the created cell suspension into a sterile 50 ml centrifuge tube and place in the centrifuge for 3-5 minutes at 220 g to form a cell pellet.
Remove from the centrifuge and aspirate the solution to leave only the cell pellet.
The cells will be expanded at 3:1 so 36 ml of prepared Keratinocyte Growth Medium (C2011B) containing Keratinocyte Supplement Mix (C39016) and CaCl (C34005) should be added and gently mixed to put the cells back into suspension.
Prepare 3 new sterile culture flasks, noting the cell line and passage number and pipette an equal (12 ml) amount of the cell suspension into each and return to the incubator.

Experimental phase: When the appropriate quantity of cells were grown, they were detached as described above, but at the final step were seeded into each well of one or more 6 well dishes. Each well received 2 ml of the cells suspended in suspension so they were seeded equally. The dishes were returned to the incubator until 85-95% confluency is reached.

It was at this stage that all wells were rinsed in PBS after aspirating the growth media. The compound(s) to be tested were previously mixed into a suitable solvent (e.g., DMSO or ethanol) that can be diluted to the desired concentration of test compound (10 μl) in the culture wells without exceeding maximum solvent concentrations and adversely effecting cell viability, and mixed with Basal Keratinocyte Media. NOTE: Standard maximum levels of solvent are 0.1% for DMSO and 0.5% for ETOH. The concentration of each test compound is listed in Tables 3 and 4. The concentration for each tested compound/natural extract is generally listed in the left-hand column of Table 4. For certain compositions (e.g., compositions 15 and 16), other concentrations were used and are noted in the body of Table 4.

Every 2 wells on the 6 well plate served as a biological replicate for the purposes of RNA isolation and genetic expression evaluations making each 6 well dish an n=3 for the test compound contained in the media. Negative control plates (no test compound and only basal media) were also generated. The plates were returned to the incubator for 24 hrs.

At the end of the 24 hr time period, the plates were removed from the incubator, the test media aspirated, and the cells rinsed with PBS. RNA isolation was performed using the BioRad Aurum Total RNA Mini Kit (Hercules, Calif.) per manufacturer's protocol; which is described, in brief, below:
Add 350 μl of lysis buffer to each well. Pipet multiple times to ensure thorough lysis. Add lysed cells to the collection tube.
Add 350 μl of 70% ETOH to the collection tube and mix thoroughly.
Pipet the generated lysate onto the spin column placed in a new 2 ml tube. Centrifuge for 30 sec between 8,000 and 10,000 g.
Place the spin column containing the now bound RNA, in a new 2 ml tube. Add 700 μl of low stringency wash solution to the spin column. Centrifuge for 30 sec between 8,000 and 10,000 g. Discard the flow through and replace spin column.
Add 80 μl of DNase I dilution to the spin column and incubate at room temperature for 15 minutes.
Add 700 μl of high stringency wash solution to the column and centrifuge for 30 sec between 8,000 and 10,000 g. Discard flow through.
Add 700 μl of low stringency wash solution to the column and centrifuge for 60 sec between 8,000 and 10,000 g. Discard flow through.
Centrifuge for 2 minutes to remove residual wash solution.
Transfer spin column to new 1.5 ml collection tube and add 80 μl of elution solution to the column. Allow 1 minute for saturation of the membrane and then centrifuge for 2 minutes to complete the RNA elution.
Quantify the quantity of RNA and store at −20 C° until use (no more than 1 month)

RNA is quantified by Optical Density readings at 260 and 280 nm using a DeNovix DS11+ spectrophotmeter. A 260/280 ratio of −2.0 is generally accepted as “pure” for RNA and is used to determine if a sample is of sufficient quality to be used to generate viable gene expression data.

List of test compounds: compounds and compositions tested are indicated in tables 3 and 4 including β caryophyllene, honokiol, magnolol, curcumin, triptolide, N-palmitoylethanolamide, triterpene alcohols & triterpendiol monoesters (faradiol), 7-hydroxyflavone, 3,7-dihydroxyflavone, N-oleoylethanolamide, disophenol, isomenthone, menthone, limonene, docosahexaenoic acid (DHA), eicosapentaenoic acid (EPA), epigallocatechin gallate, and apigenin and for certain compositions thereof. Concentrations of test compounds is found in tables 3 and 4

The genes tested were:
Collagen, Type I, Alpha-1 (COL1A1)
Integrin, Beta-1 (ITGB1)
Jun proto-oncogene, AP-1 transcription factor (JUN)
Kruppel-Like Factor 4 (KLF4)
Ceramide Synthase 3 (CERS3)
Filaggrin (FLG)
Toll-Like Receptor 2 (TLR2)
Interleukin 1-Alpha (ILIA)
Fibroblast Growth Factor 7 (FGF7)
Nuclear Factor Kappa-B; Subunit 1 (NFKB1)
Matrix Metalloproteinase 1 (MMP1)
Hemoglobin Subunit Beta (HBB) REFERENCE GENE
Ribosomal Protein L13a (RPL13A) REFERENCE GENE

The gene descriptions are:
COL1A1—Collagen, type I, alpha 1, also known as alpha-1 type I collagen, is a protein that in humans is encoded by the COL1A1 gene. COL1A1 encodes the major component of type I collagen, the fibrillar collagen found in most connective tissues, including cartilage.
ITGB1—Integrin beta-1 (ITGB1), is a cell surface receptor that in humans is encoded by the ITGB1 gene. This integrin associates with integrin alpha 1 and integrin alpha 2 to form integrin complexes which function as collagen receptors. Integrin family members are membrane receptors involved in cell adhesion and recognition in a variety of processes including embryogenesis, hemostasis, tissue repair, immune response and metastatic diffusion of tumor cells. Integrins link the actin cytoskeleton with the extracellular matrix and they transmit signals bidirectionally between the extracellular matrix and cytoplasmic domains.
JUN c-Jun is a protein that in humans is encoded by the JUN gene. c-Jun, in combination with c-Fos, forms the Activator protein 1 (AP-1) early response transcription factor that regulates gene expression in response to a variety of stimuli, including cytokines, growth factors, stress, and bacterial and viral infections. AP-1 controls a number of cellular processes including differentiation, proliferation, and apoptosis.
KLF4—KLF4 is involved in the regulation of cellular proliferation, differentiation, apoptosis, and somatic cell reprogramming. Evidence also suggests that KLF4 is a tumor suppressor in certain cancers. In embryonic stem cells (ESCs), KLF4 has been demonstrated to be a good indicator of stem-like capacity. KLF4 has diverse functions, and some of its functions are apparently contradicting, but mainly since the discovery of its integral role as one of four key factors that are essential for inducing pluripotent stem cells. KLF4 is highly expressed in non-dividing cells and its overexpression induces cell cycle arrest. KLF4 is particularly important in preventing cell division when the DNA is damaged. KLF4 is also important in regulating centrosome number and chromosome number (genetic stability), and in promoting cell survival. However, some studies have revealed that under certain conditions KLF4 may switch its role from pro-cell survival to pro-cell death.
CERS3—Ceramide synthase is an enzyme encoded by the CERS3 gene, that catalyzes the synthesis of C24 ceramide.
FLG—Filaggrin (filament aggregating protein) is a filament-associated protein that binds to keratin fibers in epithelial cells. Ten to twelve filaggrin units are post-translationally hydrolyzed from a large profilaggrin precursor protein during terminal differentiation of epidermal cells. In humans, profilaggrin is encoded by the FLG gene.
TLR2—Toll-like receptor 2 also known as TLR2 is a protein that in humans is encoded by the TLR2 gene. TLR2 plays a role in the immune system. TLR2 is a membrane protein, a receptor, which is expressed on the surface of certain cells and recognizes foreign substances and passes on appropriate signals to the cells of the immune system.
IL1a—Interleukin 1 alpha (IL-1α) also known as hematopoietin 1 is a cytokine of the interleukin 1 family that in humans is encoded by the ILIA gene. In general, Interleukin 1 is responsible for the production of inflammation, as well as the promotion of fever and sepsis. IL-1α is produced mainly by activated macrophages, as well as neutrophils, epithelial cells, and endothelial cells. It possesses metabolic, physiological, haematopoietic activities, and plays one of the central roles in the regulation of the immune responses. It binds to the interleukin-1 receptor. It is on the pathway that activates tumor necrosis factor-alpha.
FGF7/KGF: Fibroblast Growth Factor/Keratinocyte Growth Factor has a mitogenic effect on epithelial cells, but primarily in keratinocytes. There is little to no activity noted in fibroblasts or endothelial cells. FGF family members are key regulators of cell survival and have roles in a multitude of biological processes like tumor growth/invasion, cell growth, tissue repair, morphogenesis, and embryonic development. FGF7/KGF is thought to be a factor in the mesenchymal stimulation of normal epithelial tissue proliferation. Thought to play a role in hair development and wound re-epithelialization.
NFKB—NF-κB (nuclear factor kappa-light-chain-enhancer of activated B cells) is a protein complex that controls transcription of DNA, cytokine production and cell survival. NF-κB is found in almost all animal cell types and is involved in cellular responses to stimuli such as stress, cytokines, free radicals, heavy metals, ultraviolet irradiation, oxidized LDL, and bacterial or viral antigens. NF-κB plays a key role in regulating the immune response to infection.
MMP1—Matrix metalloproteinase-1 (MMP-1) also known as interstitial collagenase and fibroblast collagenase is an enzyme that in humans is encoded by the MMP1 gene that breaks down collagen.

Performance of custom microarray: Custom microarrays were performed in accordance with the protocol set forth in the Performance of custom microarray section in Example 1.

Custom microarray analysis: Analysis was carried out in accordance with the protocol set forth in the Custom Microarray analysis section of Example 1. See Example 1 for Objectives 1-3 of the analysis.

Objective 4—Identify comparative efficacy of gene response for all compounds and compositions tested vs. equivalent concentrations of CBD.

Results: See Tables 3 and 4 for individual ingredients and compositions test results, including concentrations of the compounds used therein; composition ingredient key follows:

Composition 6: curcumin, B-caryophyllene, N-palmitoylethanolamide, honokiol/magnolol, docosahexaenoic acid (DHA), eicosapentaenoic acid (EPA), triptolide
Composition 7: curcumin, B-caryophyllene, N-palmitoylethanolamide, honokiol/magnolol, epigallocatechin gallate, docosahexaenoic acid (DHA), eicosapentaenoic acid (EPA), triptolide
Composition 8: curcumin, B-caryophyllene, N-palmitoylethanolamide, honokiol/magnolol, epigallocatechin gallate, apigenin, docosahexaenoic acid (DHA), eicosapentaenoic acid (EPA), triptolide
Composition 9: curcumin, B-caryophyllene, N-palmitoylethanolamide, docosahexaenoic acid (DHA), eicosapentaenoic acid (EPA), triptolide
Composition 10: curcumin, B-caryophyllene, N-oleoylethanolamine, N-palmitoylethanolamide, 7-hydroxyflavone, docosahexaenoic acid (DHA), eicosapentaenoic acid (EPA), triptolide
Composition 11: curcumin, B-caryophyllene, N-palmitoylethanolamide, epigallocatechin gallate, docosahexaenoic acid (DHA), eicosapentaenoic acid (EPA), triptolide
Composition 12: curcumin, B-caryophyllene, N-oleoylethanolamine, N-palmitoylethanolamide, 7-hydroxyflavone, epigallocatechin gallate, docosahexaenoic acid (DHA), eicosapentaenoic acid (EPA), triptolide
Composition 13: curcumin, B-caryophyllene, N-oleoylethanolamine, N-palmitoylethanolamide, (Daemonorops draco), epigallocatechin gallate, docosahexaenoic acid (DHA), eicosapentaenoic acid (EPA), triptolide
Composition 14: curcumin, B-caryophyllene, N-oleoylethanolamine, N-palmitoylethanolamide, (Daemonorops draco), epigallocatechin gallate, docosahexaenoic acid (DHA), eicosapentaenoic acid (EPA), triptolide
Composition 15: curcumin, B-caryophyllene, N-palmitoylethanolamide, diosphenol, isomenthone, menthone, limonene (Bucha Extract), docosahexaenoic acid (DHA), eicosapentaenoic acid (EPA), triptolide
Composition 16: curcumin, B-caryophyllene, N-palmitoylethanolamide, (Daemonorops draco), diosphenol, isomenthone, menthone, limonene (Bucha Extract), epigallocatechin gallate, docosahexaenoic acid (DHA), eicosapentaenoic acid (EPA), triptolide

TABLE 3
(Desired Gene Direction, i.e., ↑ = upregulation, ↓ = downregulation/Gene)

| | ↑CERS3 | ↑COL1A1 | ↑FLG | ↓IL1A | ↑ITGB1 | ↓JUN | ↑KLF4 | ↓MMP1 | ↓NFKB1 | ↑TLR2 |
|---|---|---|---|---|---|---|---|---|---|---|
| 20 ppm CBD | 1.7 | 1.6 | 1.1 | 2.3 | 1.8 | 2.7 | 3.5 | 6.5 | 2.5 | 2.8 |
| [10 uM = 31 ppm] | | | | | | | | | | |
| Composition 8 | 1.9 | 3.1 | 6.1 | −1.4 | 2.2 | −1.4 | 6.6 | 53.9 | 6.5 | 2.6 |
| Composition 7 | −1 | 3.7 | 16.3 | −1.6 | −1.3 | 3.3 | 17.8 | 12.7 | 4.7 | −1.1 |
| Composition 6 | −1.2 | −1.4 | 4.8 | −2.8 | 2 | −5.8 | 1.1 | 92.3 | 1.3 | 1.1 |
| Composition 9 | −3 | −3.4 | 4.9 | −8.3 | 1.5 | −10.7 | −11.6 | 31.6 | −13.4 | −27.8 |
| Composition 11 | −8.3 | −6.6 | 1.3 | −5 | 2.4 | −18.5 | −48.8 | 21.9 | −6.4 | −19.9 |
| Composition 10 | −2.5 | −2.8 | 4.6 | −9.5 | 1.3 | −12 | −22.5 | 28.1 | −7.2 | −5.1 |
| Composition 12 | −11.6 | −2.2 | −4.5 | −2.7 | 5.5 | −7.1 | −10.3 | 8.5 | −1.2 | −2 |
| Composition 13 | −9.5 | −1.8 | −7.6 | −1.4 | 5.2 | −9.1 | −29.8 | 11.4 | −1.1 | −5.7 |
| Composition 14 | −19.1 | −2.3 | −10 | −2.7 | 3.9 | −8.2 | −31.2 | 5.5 | −1.5 | −4.4 |
| Composition 15 | −2.4 | −1.4 | −1.1 | −9.8 | 3.2 | −5.2 | −3.7 | 9.8 | −1.9 | −2.6 |
| 10 uM* | | | | | | | | | | |
| Composition 16 | −6 | −3.7 | −7.7 | 1.2 | 3.2 | −4 | −6 | 7.5 | −1.3 | −10.9 |
| 10 uM* | | | | | | | | | | |

DIRECT ECS

20 ppm Curcumin

INDIRECT ECS

| | | | | | | | | | | |
|---|---|---|---|---|---|---|---|---|---|---|
| 20 ppm N- | 2 | −2 | 28.6 | −1.2 | −4 | −1.9 | 1.7 | 12.2 | −2.2 | −1.3 |
| palmitoylethanolamide | | | | | | | | | | |
| 20 ppm N- | | | | | | | | | | |
| Oleylethanolamide | | | | | | | | | | |

ECS INFLAMMATORY NUCLEAR

| | | | | | | | | | | |
|---|---|---|---|---|---|---|---|---|---|---|
| Disophenol | | | | | | | | | | |
| (Bucha Extract) | | | | | | | | | | |
| 20 ppm Honokiol/ | 4.3 | 2.5 | 20.9 | 3.1 | 1.2 | 1.8 | 4.6 | 13.7 | 2 | 4.3 |
| Magnolol | | | | | | | | | | |

ECS INFLAMMATORY ENZYMATIC

20 ppm B-
Caryophyllene
20 ppm EGCG

ECS INFLAMMATORY CYTOKINE

| | | | | | | | | | | |
|---|---|---|---|---|---|---|---|---|---|---|
| 20 ppm 7- | | | | | | | | | | |
| Hydroxyflavone | | | | | | | | | | |
| 20 ppm Dragons Blood | | | | | | | | | | |
| Extract | | | | | | | | | | |
| 20 ppm Apigenin | | | | | | | | | | |
| 20 ppm Marigold Ext | 2.3 | 1.2 | 4.4 | 2.3 | 1.4 | −1.5 | −1.3 | 11.7 | −1.3 | −1 |
| 20 ppm Triptolide | −1.5 | −1.3 | 14.4 | −14 | −14.7 | −4.7 | 1.5 | 1.2 | −4.2 | −2.9 |

ECS TRP PATHWAY

| | | | | | | | | | | |
|---|---|---|---|---|---|---|---|---|---|---|
| 20 ppm Algal Oil | 2.1 | −1.9 | 27.6 | −1.2 | −3.5 | −1.5 | 1.7 | 8.5 | −1.8 | 1.1 |

TABLE 4

| COMP | COMP | COMP | COMP | COMP | COMP | COMP | COMP | COMP | COMP | COMP |
|---|---|---|---|---|---|---|---|---|---|---|
| 6 | 7 | 8 | 9 | 10 | 11 | 12 | 13 | 14 | 15 | 16 |

DIRECT ECS

| | | | | | | | | | | | |
|---|---|---|---|---|---|---|---|---|---|---|---|
| 20 ppm Curcumin | x | x | x | x | x | x | x | x | 40 ppm | 10 uM | 10 uM |

INDIRECT ECS

| | | | | | | | | | | | |
|---|---|---|---|---|---|---|---|---|---|---|---|
| 20 ppm N- | x | x | x | x | x | x | x | x | x | 10 uM | 10 uM |
| palmitoylethanolamide | | | | | | | | | | | |
| 20 ppm N- | | | | | x | | x | x | x | | |
| Oleylethanolamide | | | | | | | | | | | |

ECS INFLAMMATORY NUCLEAR

| | | | | | |
|---|---|---|---|---|---|
| Disophenol | | | | 10 uM | 10 uM |
| (Bucha Extract) | | | | | |
| 20 ppm Honokiol/ | x | x | x | | |
| Magnolol | | | | | |

ECS INFLAMMATORY ENZYMATIC

| | | | | | | | | | | | |
|---|---|---|---|---|---|---|---|---|---|---|---|
| 20 ppm B- | x | x | x | x | x | x | x | x | x | 10 uM | 10 uM |
| Caryphyllene | | | | | | | | | | | |
| 20 ppm EGCG | | x | x | | | 50 ppm | 50 ppm | 50 ppm | 50 ppm | | 10 uM |

ECS INFLAMMATORY CYTOKINE

| | | | | | | | | | | | |
|---|---|---|---|---|---|---|---|---|---|---|---|
| 20 ppm 7- | | | | | x | | x | | | | |
| Hydroxyflavone | | | | | | | | | | | |
| 20 ppm Dragons | | | | | | | | x | x | | 23 ppm |
| Blood Extract | | | | | | | | | | | |
| 20 ppm Apigenin | | | x | | | | | | | | |
| 20 ppm Marigold Ext | | | | | | | | | | | |
| 20 ppm Triptolide | x | x | x | x | x | x | x | x | 40 ppm | 10 uM | 10 uM |

ECS TRP PATHWAY

| | | | | | | | | | | | |
|---|---|---|---|---|---|---|---|---|---|---|---|
| 20 ppm Algal Oil | x | x | x | x | x | x | x | x | x | 10 ppm | 10 ppm |

Table 3 depicts fold increases or decreases in gene expression of each listed gene following treatment of cells with the listed compound, natural extract, or composition (i.e. a combination of compounds, natural extracts, or a combination thereof) compared to gene expression of the same gene in untreated cells. Table 4 indicates the compounds and natural extracts, shown by an “x,” contained within each tested composition. The concentration of the compound or natural extract as tested is indicated on the Tables 3 and 4 as either uM or ppm. In the case of a tested composition, the concentration of each compound or natural extract within the composition is equivalent to its standalone test concentration, unless otherwise indicated. In other words, if compound X was tested at 10 uM individually, then compound X is present as a component of a tested composition at 10 uM concentration.

For a combination of at least one direct endocannabinoid compound, a second indirect endocannabinoid compound, a third ECS related pathway anti-inflammatory compound consisting of one or more compounds targeting specific anti-inflammatory pathways, e.g. nuclear, enzymatic, or cytokine pathways, a fourth compound targeting any one of the ECS related TRP pathways targeting keratinocyte skin matrix pathways, cellular proliferation, differentiation, autophagy, apoptosis, and senescence, barrier function and skin microbiome, and optionally a fifth compound that affects a pathway not affected by any of the preceding compounds are identified that indicate a beneficial profile for mitigating inflammation, and improving skin matrix and wound healing. One or more of the identified combinations demonstrate (a) a synergistic gene expression effect in comparison to the gene expression elicited by the compounds of the combination(s) individually (see, e.g., the bolded results in Table 3), and/or (b) a greater increase or decrease in gene expression in comparison to a composition comprising equivalent concentration of cannabidiol.

Specifically for composition 8 the combination of compounds designed for anti-inflammatory, anti-aging, skin matrix improvement, and wound healing selected from the groups consisting of direct and indirect endocannabinoid compounds, anti-inflammatory compounds from each of the three ECS related anti-inflammatory pathways (nuclear, enzymatic, and cytokine), and the ECS related TRP pathway compounds targeting skin matrix and barrier pathways, includes curcumin, B-caryophyllene, N-palmitoylethanolamide, honokiol, magnolol, epigallocatechin gallate, apigenin, docosahexaenoic acid (DHA), eicosapentaenoic acid (EPA), triptolide produced synergistic beneficial gene test results for COL1A, ITGB1 and KLF4 (see composition 8). The group designation by chemical class and compound identification for the composition is:
Direct ECS pathway (CB1, CB2): Curcuminoids: curcumin
Indirect ECS pathway (FAAH, MAGL): Fatty Acid Amides: N-palmitoylethanolamide
ECS related Anti-inflammatory nuclear pathway (PPARg): Biphenols: honokiol, magnolol
ECS related Anti-inflammatory enzymatic pathway (PTGS1): Sesquiterpenes: B-caryophyllene; Flavan-3-ols: EGCG
ECS related Anti-inflammatory cytokine pathway: (ILIA, NFKB): Diterpenes: Triptolide Hydroxyflavones: Apigenin
ECS related TRP Pathway: (TRPV1, TRPA1, TRPM8): PUFAs: docosahexaenoic acid (DHA), eicosapentaenoic acid (EPA)

Additionally, this compositions comprised from the selection of at least one compound from each of the chemical classes comprising curcuminoids, fatty acid amides, sesquiterpenes, flavan-3-ols, diterpenes, hydroxyflavones and PUFAs demonstrated beneficial skin matrix gene modulation for the genes COL1A1 (+), ITGB1 (+), JUN (−) and KLF4 (+) and beneficial skin barrier gene modulation for the genes CERS3 (+), FLG (+), and TLR2 (+).

Summarily the findings for composition 8 were:
Superior gene expression response compared cannabidiol CBD for CERS3, COL1A1, FLG, ILIA, ITGB1, JUN, & KLF4
Synergistically superior to individual composition compounds in gene response for COL1A1, ITGB1, & KLF4
Strong increase in MMP associated consistent with initial wound healing expectations
100% more effective agonist for COL1A1 and KLF4 than cannabidiol

The results of the experiments carried out in Examples 1-3 are summarized in the following tables 5, 6 and 7. Additionally, in certain cases, the tables identify additional compounds and natural extracts suitable for the invention.

TABLE 7
CURCUMINOIDS: curcumin*, demethoxycurcumin, bisdemethoxycurcumin (Curcuma longa,
Curcuma xanthorrhiza, Curcuma zedoaria), tetrahydrocurcumin*
ALLYL CHAIN SUBSTITUTED GUAIACOLS: eugenol* (Eugenia caryophyllata, Syzygium
aromaticum, Myristica fragrans, Cinnamomum verum, Ocimum basilicum, Laurus nobilis),
and its isomers and derivatives including isoeugenol, dihydroeugenol, and ethyl guaiacol
FATTY ACID AMIDES: N-palmitoylethanolamide (PEA)* (Glycine max, Arachis hypogaea,
Gallus gallus domesticus (egg oil)) , N-oleoylethanolamide (OEM* (Theobroma cacao,
Achyranthes aspera), Stearoylethanolamide (SEA), N-arachidonylethanolamide (AEA),
Linoleoylethanolamide, Oleamide, Arachidonamide
GINSENOSIDES: Compounds in this family are found almost exclusively in the plant
genus Panax (ginseng) i.e. (ginsenosides or panaxosides) are a class of natural
product steroid glycosides and triterpene saponins including ginsenoside RC* (Panax
notogensing (root), Panex gensing)
MONOTERPENES: disophenol*, isomenthone*, menthone*, limonene* (Agathosma
betulina, Agathosma crenulata), menthol* (Mentha longifolia), myrcene (Syzygium
polyanthum, Laurus nobilis, Humulus lupulus), linalool (Boswellia serrata, Zingiber officinale,
Ocimum basilicum L., Citrus bergamia), pinene (Myristica fragrans, Melaleuca leucadendra L,
Boswellia serrata, Artemisia californica), camphor* (Artemisia californica, Cinnamomum
camphora).
BIPHENOLS: Honokiol*, Magnolol/Honokiol (50/50)* (Magnolia officinalis, Magnolia
grandiflora, Magnolia dealbata, Magnolia biondii, Magnolia obovata), and stilbenoids
including resveratrol (Vitis Vinifera L., Vaccinium sp.) and diethylstilbestrol
SESQUITERPENES: β Caryophyllene* (see below), humulene (Humulus lupulus), farnesene
(Humulus lupulus, Curcuma longa, Curcuma xanthorrhiza, Curcuma zedoaria), zingiberene
(Zingiber officinale), longifolene (Pinus longifolia, Pinus roxburghii), copaene (Copaifera
langsdorfii, Citrus aurantiifolia, Citrus reticulata) and the alcohol patchoulol (Pogostemon
cablin).
TERPENE LACTONES: Ginkolide B*, A, C, J, & M, bilobalide (Ginkgo biloba),
parthenolide (Tanacetum parthenium), helenalin (Arnica montana), lactucin, lactucopicrin
(Lactuca virosa)
FLAVAN-3-OLS: Epigallocatechin gallate* (Camellia sinensis,
Helianthemum glomeratum, Vaccinium oxycoccos, Fragaria ananassa, Rubus fruticosus,
Actinidia deliciosa, Prunus avium, Pyrus sp., Prunus persica, Malus domestica, Persea
americana, Carya illinoinensis, Pistacia vera, and Corylus avellana.), catechin, epicatechin,
gallocatechin, epigallocatechin, catechin gallate, epicatechin gallate, epiafzelechin,
fisetinidol, guibourtinidol, mesquitol, robinetinidol.
NAC: N-Acetyl L-Cystene
HYDROXYFLAVONES: 7-hydroxyflavone*, 3,7-dihydroxyflavone* (Daemonorops draco*,
Dracaena cochinchinensis), quercetin (Camellia sinensis), fisetin (Fragaria sp.), apigenin*
(Matricaria chamomilla, Petroselinum crispum, Allium cepa, Citrus Sinensis, Triticum
aestivum), kaempferol (Brassica sp., Spinacia sp.)
DITERPENES: Triptolide* (Tripterygium wilfordii), Rosmanol (Salvia mellifera), Carnosic
acid (Salvia mellifera, Rosmarinus officinalis, Salvia officinalis), Salvinorin A
(Salvia divinorum), Forskolin
TRITERPENES: Triterpene alcohols & Triterpendiol monoesters (Faradiol) (Calendula
officinalis)*
N-ALKYLAMIDES (NAAs): dodeca-2E,4E,8Z,10Z-tetraenoic acid isobutylamide and
dodeca-2E,4E-dienoic acid isobutylamide (Echinacea purpurea extract)*
PUFAs: Eicosapentaenoic acid (EPA), Docosahexaenoic acid (DHA), Algal oil*,
(Schizochytrium aggregatum), Clupea pallasii (Pacific Herring oil), Oncorhynchus
tshawytscha (Chinook Salmon oil), Euphausia sp. (krill oil)), Alpha-Linolenic Acid
(ALA) (Linum usitatissimum, Camelina sativa, Perilla frutescens, Juglans nigra),
Eicosatetraenoic Acid (ETA), Oleic Acid (Olea europaea), Palmitoleic Acid (macadamia
integrifolia), Vaccenic Acid.
β Caryophyllene (Bidens pilosa, Syzygium aromaticum (Eugenia caryophyllata), Piper
nigrum, Perilla frutescens, Rosmarinus officinalis, Lindera benzoin, Centella asiatica,
Angelica archangelica, Coleus barbatus, Origanum vulgare, Ptychopetalum olacoides,
Ocimum basilicum, Salvia officinalis, Vitex agnus-castus, Petroselinum crispum,
Coriandrum sativum, Boswellia sacra, Apium graveolens, Eucalyptus citriodora, Piper
cubeba, Cinnamomum verum, Thymus vulgaris, Myrrhis odorata, Pinus sylvestris,
Valeriana officinalis, Aesculus hippocastanum, Murraya koenigii, Tagetes minuta,
Tamarindus indica, Melaleuca alternifolia, Mentha longifolia, Citrus limon, Ocimum
tenuiflorum, Tagetes filifolia, Hedychium flavum, Eucalyptus tetraptera, Micromeria
fruticosa, Salvia triloba, Artemisia annua, Salvia canariensis, Pogostemon cablin, and
Copaifera officinalis.)
(*denotes compounds and natural extracts that were tested)

### Example 4

Cell model: A reconstructed human skin equivalent model (HSE) is obtained through MatTek Corporation (Ashland, Mass.). The planned initial culture is the EpiDermFT™ model (EFT-400) which is comprised of Normal human epidermal keratinocytes (NHEK) and Normal human dermal fibroblasts (NHDF) from the following locations from an adult donor:

NEHK: Adult breast skin
NHDF: Adult skin

The cells are co-cultured until they have organized into 8-12 cell layers with a fully developed stratum corneum including (basal, spinous and granular layers). These cell models are in discreet wells of a culture dish grown on tranpsore membranes that allow nutrient media to feed the model without submerging it in media as in other forms of cell culture.

Culture media: Cells are provided from the manufacturer ready to use and containing relevant media. This media is proprietary and is provided by the manufacturer with purchase and is composed of Dulbecco's Modified Eagle's Medium (DMEM) supplemented with Gentamicin 5 Amphotericin B 0.25 μg/ml, Phenol red, proprietary lipid precursors used to enhance epidermal barrier formation, epidermal growth factor, insulin, hydrocortisone and other proprietary stimulators of epidermal differentiation. During the 24 hour experimental phase, cells are maintained in only the basal medium which has the test compound(s) added. All cultures will be incubated at 37° C. with 5% CO₂ in a humidified chamber.

Cell culture growth and expansion phase: The selected HSE models do not need to be expanded and will be provided from the manufacturer ready to use.

Experimental phase: When cultures arrive, they are ready to use and after an equilibration period will enter directly into the experimental phase. It is at this stage that the models are rinsed with PBS after aspirating the growth media. The compound(s) to be tested were previously mixed into a suitable solvent (e.g., DMSO or ethanol) that can be diluted to the desired concentration of test compound (10 μl) in the culture wells without exceeding maximum solvent concentrations and adversely effecting cell viability, and mixed with basal media. NOTE: Standard maximum levels of solvent are 0.1% for DMSO and 0.5% for ETOH. Alternately, the HSE models allow for the test compound to be applied topically to the model using an appropriate solvent without being mixed into the basal media at all. In this case the test compound would be applied directly to the HSE model; the model would still be placed in the basal medium for the 24 hr test period.

Every well on the 6 well plate can potentially serve as a biological replicate for the purposes of RNA isolation and genetic expression (depending on the expected RNA yield) evaluations making each 6 well dish an n=6 for the test compound contained in the media or topically applied. Since the expected RNA yield is expected to be much higher than monolayer cell culture, it is fully expected that one 6 well plate of HSE will be the equivalent of 6 individual exposures of the designated experimental condition. Negative control plates (no test compound(s) and only basal media) are also generated. The plates are returned to the incubator for 24 hrs.

At the end of the 24 hr time period, the plates are removed from the incubator, the transwell membrane containing the HSE model is removed from the and the rinsed with PBS. RNA isolation will be performed using the BioRad Aurum Total RNA Mini Kit (Hercules, Calif.) per manufacturer's protocol; which is described, in brief, below:
Remove the HSE model from the transpore membrane.
Add 700 μl of lysis buffer to the collection tube.
Add the HSE model to the same collection tube.
Use a rotor-stator homogenizer for 30-60 seconds to disrupt the HSE model.
Add 700 μl of 60% ETOH to the collection tube and mix thoroughly.
Pipet the generated lysate onto the spin column placed in a new 2 ml tube. Centrifuge for 30 sec between 8,000 and 10,000 g.
Place the spin column containing the now bound RNA, in a new 2 ml tube. Add 700 μl of low stringency wash solution to the spin column. Centrifuge for 30 sec between 8,000 and 10,000 g. Discard the flow through and replace spin column.
Add 80 μl of DNase I dilution to the spin column and incubate at room temperature for 15 minutes.
Add 700 μl of high stringency wash solution to the column and centrifuge for 30 sec between 8,000 and 10,000 g. Discard flow through.
Add 700 μl of low stringency wash solution to the column and centrifuge for 60 sec between 8,000 and 10,000 g. Discard flow through.
Centrifuge for 2 minutes to remove residual wash solution.

Transfer spin column to new 1.5 ml collection tube and add 80 μl of elution solution to the column. Allow 1 minute for saturation of the membrane and then centrifuge for 2 minutes to complete the RNA elution. Quantify the quantity of RNA and store at −20 C° until use (no more than 1 month)

RNA is quantified by Optical Density readings at 260 and 280 nm using a DeNovix DS11+ spectrophotmeter. A 260/280 ratio of −2.0 is generally accepted as “pure” for RNA and will be used to determine if a sample is of sufficient quality to be used to generate viable gene expression data.

List of test compounds: Compounds are selected from those outlined in the Scaled Gene Function by Chemical Class, Compound and Extracts in Tables 5, 6, and 7.

Performance of custom microarray: The gene expression data is generated by utilizing the isolated RNA samples in custom designed cDNA microarrays in a 96 well format. The arrays are set up to test duplicates of up to 11 genes of interest with 2 housekeeping/reference genes for up to 4 biological replicates on each plate. The array layout is shown in Example 1.

The genes of interest are listed in Examples 1, 3, 5 and 6. The remaining wells serve as assay controls for genomic DNA contamination, Polymerase reaction efficiency and transcription rates.

The arrays are performed by using equal amounts of sample RNA from each of the tested compounds to be amplified using the BioRad iScript cDNA synthesis kit (Hercules, Calif.) per manufacturer's instructions. Briefly this consists of taking the designated amount of starting RNA and mixing it with the required amount of synthesis buffer/reverse transcriptase and performing a series of amplification reactions (5 min 25 C° priming; 20 min 46 C° reverse transcription and 1 min 95 C° reverse transcription inactivation) to generate the cDNA needed for the array.

This cDNA template is mixed with enough BioRad SsoAdvanced Universal SYBR Supermix (Hercules, Calif.) to generate enough sample for the 96 well plate (20 μl per well). The cDNA serves as the template to the specific gene primers in each of the wells which undergo polymerase chain reaction (PCR) to amplify the gene marker contained in that well. A typical PCR reaction consists of Denaturing, and Annealing/Extension steps repeated for approximately 40 cycles. As these genes are amplified, the SYBR mix gives of a fluorescence which is detected by the BioRad iCycler CFX Touch (Hercules, Calif.) system in real time. This fluorescence eventually breaks a basal level known as the background level. The cycle at which these levels are broken relative to the reference genes and the levels of an untreated control sample determine the fold increase or decrease of the gene expression seen in cells treated by the tested compounds.

Custom microarray analysis: Analysis is performed in accordance with the analysis protocol set forth in Example 1.

### Example 5

Cell cultures: The cell cultures are selected from those set forth in Example 1.

Culture media: The culture media is the same as set forth in Example 1

Cell culture growth and expansion phase: The cell culture growth and expansion protocol is the same as set forth in Example 1. Experimental phase: The experimental phase protocol is the same as set forth in Example 1.

List of test compounds: Compounds are selected from those listed in tables 5, 6, and 7.

The genes to test are:
Galactoside, Beta 1 (GLB1)
Cyclin-Dependent Kinase Inhibitor 2A (CDKN2A)
Cyclin-Dependent Kinase Inhibitor 1A (CDKN1A)
Tumor Protein 53 (TP53)
MDM2 Protooncogene (MDM2)
Mitogen Activated Protein Kinase 1 (MAPK1)
Apoptosis Related Cysteine Protease; Caspase 8 (CASP8)
Hemoglobin Subunit Beta (HBB) REFERENCE GENE
Ribosomal Protein L13a (RPL13A) REFERENCE GENE

The gene descriptions are:
GLB1: A lysosomal hydrolase that can complex with Cathepsin A and Neuraminidase to form a component of cell surface receptors important in elastin binding protein and associated connective tissue. Main function is the breakdown/recycling of molecules in the cell lysosome. Primary targets are GM1 ganglioside (important in neural cell function) and the glycosaminoglycan keratan sulfate found in cartilage and the cornea.
CDKN2A: Encodes p14 and p16 which regulate 2 critical cell cycle pathways (the p53 and RB1 pathways). The RB1 protein phosphorylation is inhibited by p16, which induces G1 cell cycle arrest and tumor suppression. The p14 protein binds MDM2 which in turn stabilizes p53; this binding can also enhance p53 dependent transcription and apoptosis. Can also induce G2 cell cycle arrest by preventing cyclinB 1/CDCl₂ complex activation. Can act as a negative regulator of normal cell proliferation through interaction with CDK4 and 6.
CDKN1A: Cyclin dependent kinase inhibitor tightly controlled by p53 in response to stress which can inhibit CDK2/4 complexes and regulates cell cycle G1 progression. Also plays a role in DNA damage repair and replication. Following caspase activation can lead to apoptosis. When this gene is not present in mice, they have shown some limited regeneration of damaged/missing tissue.
TP53: Ubiquitous, major stress induced protein regulating expression of genes involved in cell cycle arrest, apoptosis, senescence DNA repair and metabolism changes. Mutations in this gene are found in almost all cancer types.
MDM2: In unstressed cells, keeps TP53 transcriptional activity inactive through ubiquination which leads to TP53 degradation. Promotes the degradation of RB1 in a similar fashion. Is part of the TRIM28/KAP1-ERBB4-MDM2 complex which links growth factor and DNA damage response pathways.
MAPK1: Also known as ERK2, vital component of signal transduction to the cell nucleus where it performs phosphorylation for indicated nuclear targets generally resulting in ribosomal transcription. Along with MAPK2 and PKC controls cell cycle (proliferation, differentiation and development) and autocrine/paracrine responses.
CASP8: Part of the proteases signaling cascade responsible for initiating apoptosis in cells designated for programmed cell death induced by FAS and other apoptotic signals. May play a role in neurodegenerative disease. Can cleave and activate many other members of the caspase family resulting in the previously described signal cascade resulting in apoptotic activity. Can play a role in limiting cellular response to inflammation through cleavage of RIPK1 (Receptor Interacting Serine/Threonine Kinase).

Performance of custom microarray: The microarray is performed in accordance with the applicable protocol set forth in Example 1.

Custom microarray analysis: The microarray analysis is performed in accordance with the applicable protocol set forth in Example 1.

### Example 6

Cell cultures: A Normal Human Epidermal Keratinocyte (NHEK) cryopreserved cell culture (or cultures) is obtained through Promocell GmbH (Heidelberg, Germany). The culture(s) selected will be from pooled adult donors initially (C-12006); if needed pooled juvenile or individual juvenile or adult donors can also be used (C-12005, C-12001 or C-12003 respectively).

Culture media: The culture media is the same as set forth in Example 3.

Cell culture growth and expansion phase: The cell culture growth and expansion protocol is the same as set forth in Example 3, noting that donor vial numbers are inserted at the time of experiment.

Experimental phase: The experimental phase protocol is the same as set forth in Example 1.

List of test compounds: Compounds are selected from those listed in tables 5, 6, and 7.

The genes to test are:
Apoptosis Related Cysteine Protease; Caspase 8 (CASP8)
Cannabinoid Receptor 1 (CB1(CNR1))
Cannabinoid Receptor 2 (CB2(CNR2))
Galactoside, Beta 1 (GLB1)
Mitogen Activated Protein Kinase 1 (MAPK1)
Nuclear Factor Kappa-Beta; Subunit 1 (NFKB1)
Tumor Protein 53 (TP53)
Transient Receptor Potential Cation Channel, Subfamily A, Member 1 (TRPA1)
Transient Receptor Potential Cation Channel, Subfamily M, Member 8 (TRPM8)
Transient Receptor Potential Cation Channel, Subfamily V, Member 1 (TRPV1)
Transient Receptor Potential Cation Channel, Subfamily V, Member 3 (TRPV3)
Hemoglobin Subunit Beta (HBB) REFERENCE GENE
Ribosomal Protein L13a (RPL13A) REFERENCE GENE

The gene descriptions are:
CASP8: Part of the proteases signaling cascade responsible for initiating apoptosis in cells designated for programmed cell death induced by FAS and other apoptotic signals. May play a role in neurodegenerative disease. Can cleave and activate many other members of the caspase family resulting in the previously described signal cascade resulting in apoptotic activity. Can play a role in limiting cellular response to inflammation through cleavage of RIPK1 (Receptor Interacting Serine/Threonine Kinase).
CB1 (CNR1): Cannabinoid receptor type 1 (CB1), also known as cannabinoid receptor 1, is a G protein-coupled cannabinoid receptor that in humans is encoded by the CNR1 gene. The human CB1 receptor is expressed in the peripheral nervous system and central nervous system. It is activated by: endocannabinoids, a group of retrograde neurotransmitters that include anandamide and 2-arachidonoylglycerol (2-AG); plant phytocannabinoids, such as the compound THC which is an active ingredient of the psychoactive drug cannabis. The primary endogenous agonist of the human CB1 receptor is anandamide.
CB2 (CNR2): The cannabinoid receptor type 2 (CB2), is a G protein-coupled receptor from the cannabinoid receptor family that in humans is encoded by the CNR2 gene. It is closely related to the cannabinoid receptor type 1. The principal endogenous ligand for the CB2 receptor is 2-Arachidonoylglycerol (2-AG). The discovery of this receptor helped provide a molecular explanation for the established effects of cannabinoids on the immune system.
GLB1: A lysosomal hydrolase that can complex with Cathepsin A and Neuraminidase to form a component of cell surface receptors important in elastin binding protein and associated connective tissue. Main function is the breakdown/recycling of molecules in the cell lysosome. Primary targets are GM1 ganglioside (important in neural cell function) and the glycosaminoglycan keratan sulfate found in cartilage and the cornea.
MAPK1: Also known as ERK2, vital component of signal transduction to the cell nucleus where it performs phosphorylation for indicated nuclear targets generally resulting in ribosomal transcription. Along with MAPK2 and PKC controls cell cycle (proliferation, differentiation and development) and autocrine/paracrine responses.
NFKB: NF-κB (nuclear factor kappa-light-chain-enhancer of activated B cells) is a protein complex that controls transcription of DNA, cytokine production and cell survival. NF-κB is found in almost all animal cell types and is involved in cellular responses to stimuli such as stress, cytokines, free radicals, heavy metals, ultraviolet irradiation, oxidized LDL, and bacterial or viral antigens. NF-κB plays a key role in regulating the immune response to infection.
TP53: Ubiquitous, major stress induced protein regulating expression of genes involved in cell cycle arrest, apoptosis, senescence DNA repair and metabolism changes. Mutations in this gene are found in almost all cancer types.
TRPA1—The TRPA family is made up of 7 subfamilies, the TRPA1s have been the most extensively studied subfamily; and are believed to function as mechanical stress, temperature, and chemical sensors. TRPA1 is known to be activated by compounds such as isothiocyanate (which are the pungent chemicals in substances such as mustard oil and wasabi) and Michael acceptors (e.g. cinnamaldehyde). These compounds are capable of forming covalent chemical bonds with the protein's cysteins. Non-covalent activators of TRPA1 also exists, such as methyl salicylate, and menthol.
TRPM8—Functional TRPM channels are believed to form tetramers. The TRPM family consists of eight different channels, TRPM1-TRPM8. TRPM are activated by steroids, types include TRPM2 (inflammatory pain), TRPM3 (neurogenic pain) TRPM8: (COLD).
TRPV1: TRPV (vanilloid) also has 6 members: TRPV1: (HEAT) capsaicin, eugenol, gingerol, cannabinoids, endocannabinoids, lidocaine; inflammatory and neuropathic pain. TRPV2: CBD, probenecid; inflammatory pain. TRPV3: camphor, carvacol, thymol and AA, PUFA resolvins: inflammatory and nociceptor. TRPV4: UVB irradiation>inflammation from TRPV4 activation in keratinocytes.
TRPV3: Calcium cation channel that functions in a wide range of processes including vasoregulation and temperature sensation. May modulate activity of TRPV1. TRPV3 signaling can suppress keratinocyte proliferation, induce apoptosis, and catagen in hair follicles prematurely.

Performance of custom microarray: The microarray is performed in accordance with the applicable protocol set forth in.

Custom microarray analysis: Completed arrays are analyzed using the BioRad CFX Manager software. During the analysis four objectives are examined:

Objective 1—Compare the gene expression data from an untreated sample with any/all of the tested compound treated samples to determine fold change and p-values for every gene measured by the microarray.

Objective 2—Identify differentially expressed genes for the comparison generated in Objective 1 using standard criteria (specifically, an absolute fold change value >1.5, a log ratio p-value <0.05).

Objective 3—Identify test compounds that have the greatest fold changes, the largest number of differentially expressed genes, or a combination of both that indicates a beneficial profile for pain, inflammation and/or skin function. These compounds will inform the initial formulations for additional testing.

Objective 4—Identify comparative efficacy of gene response for all compounds tested vs. equivalent concentrations of CBD.

Compositions containing the endocannabinoid mimetic compounds described hereunder should preferably be free of sensitizing agents (e.g. parabens). Suitable compositions according to the present invention can be prepared with various ingredients, as described below.

Gel formulation for pain relief: Purified Water, Docosahexaenoic acid (DHA), Eicosapentaenoic acid (EPA), Propanediol, Egg Oil/Ovum Oil, Glycerin, Octyldodecanol, Phenoxyethanol, Barosma Betulina Leaf Oil, Acrylates/C10-30 Alkyl Acrylate Crosspolymer, Carbomer, Pentaerythrityl Tetra-di-t-butyl Hydroxyhydrocinnamate, Sodium Hydroxide, Disodium EDTA, Ethylhexylglycerin, Tripterygium wilfordii Root Extract, Pogostemon cablin Leaf Extract, Tocopherol, Tetrahydrocurcumin, Helianthus Annuus (Sunflower) Seed Oil, Curcuma Longa (Turmeric) Root Extract and optionally including an US FDA OTC Monograph External Analagesic Approved Drugs including, but not limited to, menthol, camphor, methyl salicylate or eugenol.

Anhydrous ointment formulation for pain relief: Petrolatum, Paraffin, Docosahexaenoic acid (DHA), Eicosapentaenoic acid (EPA), Egg Oil/Ovum Oil, Octyldodecanol, Tripterygium wilfordii Root Extract, Pogostemon cablin Leaf Extract, Tocopherol, Curcuma Longa (Turmeric) Root Extract, Helianthus annuus (Sunflower) Seed Oil and optionally including an US FDA OTC Monograph External Analagesic Approved Drugs including, but not limited to, menthol, camphor, methyl salicylate or eugenol.

Cream formulation for post procedure wound healing and pain modulation: Aqua, 10 CapryliclCapric Triglyceride, Bis-Hydroxyethoxypropyl Dimethicone, Glycerin, Isopropyl Lauroyl Sarcosinate, Cetearyl Glucoside, Glycine Soja Protein, Oxido Reductases, Sodium Hyaluronate, Sodium PCA, Glucose, Isohexadecane, Xanthan Gum, Cetearyl Olivate, Sorbitan Olivate, Polysorbate 20, Polysorbate 80, Hydroxyethylcellulose, Magnesium Aluminum Silicate, Steareth-100, Disodium EDTA, EGF, FGF, Oligopeptide-87, Acetyl Decapeptide-3, Nonapeptide-24, Phenoxyethanol and a endocannabinoid mimetic composition containing direct and indirect ECS compounds, ECS related pathway anti-inflammatory and skin matrix improving compounds preferably including compounds selected from the following group: curcumin, B-caryophyllene, N-palmitoylethanolamide, honokiol, magnolol, epigallocatechin gallate, apigenin, docosahexaenoic acid (DHA), eicosapentaenoic acid (EPA), triptolide, diosphenol, and eugenol, and/or plant extracts selected from: Curcuma longa (Turmeric), Aloe Vera (Aloe), Tanacetum parthenium (Feverfew), Daemonorops draco (Dragon's Blood), Tripterygium wilfordii Hook F (Thundergod), Echinacea purpurea (Echinacea), Rosmarinus officinalis (Rosemary), Lavandula sp. (Lavender), Eugenia caryophyllata (Clove), Pinus pinaster (Pine bark), Calendula officinalis (Marigold), Matricaria recutita (Chamomilla), Struthanthus vulgaris, Propolis (from bee honey).

Antiaging Serum for improving skin matrix, barrier function and balancing ECS homeostasis: Aqua, Isopropyl Lauroyl Sarcosinate, PPG-3 Benzyl Ether Myristate, Algae Extract, Glycerin, Palmitoyl Tripeptide-3, Glycerine, Phospholipids, Xanthan Gum, Glucose, Aluminum Hydroxide, Hydrated Silica, Alginic Acid, CI 77489, Silica, Cetearyl Olivate, Sorbitan Olivate, C20-22 Alkyl Phosphate, C20-22 Alcohols, Polysorbate 20, Isohexadecane, Polysorbate 80, Hydroxyethylcellulose, Triethanolamine, Disodium EDTA, Phenoxyethanol, and a endocannabinoid mimetic composition containing direct and indirect ECS compounds, ECS related pathway anti-inflammatory and skin matrix improving compounds preferably including selected from curcumin, B-caryophyllene, N-palmitoylethanolamide, triptolide, 7-hydroxyflavone, N-oleoylethanolamine, docosahexaenoic acid (DHA), and eicosapentaenoic acid (EPA) (see composition 3) and curcumin, B-caryophyllene, N-palmitoylethanolamide, honokiol/magnolol, epigallocatechin gallate, apigenin, docosahexaenoic acid (DHA), eicosapentaenoic acid (EPA), and triptolide. (see composition 8).

The following embodiments are exemplary of the present invention and should not in any way be interpreted as limiting the scope of the invention.
1. A composition comprising:
   a) at least one direct endocannabinoid mimetic compound, wherein each compound detectably or significantly modulates (preferably increases) gene expression of the CB1 and/or CB2 gene;
   b) at least one indirect endocannabinoid mimetic compound, wherein each compound:
      1) detectably or significantly modulates (preferably decreases) gene expression of FAAH; and/or
      2) detectably or significantly modulates (preferably decreases) gene expression of MAGL;
   c) at least one ECS related pathway anti-inflammatory compound, wherein each compound
      1) detectably or significantly modulates (preferably increases) gene expression of PPARgamma (PPARg), PPARalpha (PPARa), PPARbeta (PPARb), or any combination thereof; and/or
      2) detectably or significantly modulates (preferably decreases) gene expression of COX1 (i.e., PTGS1), COX2, iNOS, 5-LOX, 12-LOX, MMP1, or any combination thereof; and/or
      3) detectably or significantly modulates (preferably decreases) gene expression of IL-1beta, IL-1alpha(IL1a), IL-6, IL-8, NFKappaBeta (NFKB), TNFalpha (TNFa), and detectably or significantly modulates (preferably increases) gene expression of IL-10, or any combination thereof; and
   d) at least one ECS related TRP pathway compound, wherein each compound detectably or significantly modulates (preferably increases) gene expression of TRPA1, TRPM8, TRPV4, TRPV6 and that modulates (preferably decreases) gene expression of TRPV1, TRPV3, or any combination thereof,
      and wherein gene expression in each case is measured in a cell exposed to the compound and is compared to the gene expression in a cell not exposed to the same compound.
2. The composition of embodiment 1, wherein
   the at least one direct endocannabinoid mimetic compound is a curcuminoid or an allyl chain substituted guaiacol;
   the at least one indirect endocannabinoid mimetic compound that detectably or significantly modulates (preferably decreases) gene expression of FAAH is a fatty acid amide;
   the at least one indirect endocannabinoid mimetic compound that detectably or significantly modulates (preferably decreases) gene expression of MAGL is a ginsenoside or a fatty acid amide;
   the at least one ECS related pathway anti-inflammatory compound that detectably or significantly modulates (preferably increases) gene expression of PPARgamma (PPARg), PPARalpha (PPARa), PPARbeta (PPARb), or any combination thereof is a monoterpene;
   the at least one ECS related pathway anti-inflammatory compound that detectably or significantly modulates (preferably decreases) gene expression of COX1 (i.e., PTGS1), COX2, iNOS, 5-LOX, 12-LOX, MMP1, or any combination thereof, is a sesquiterpene, a terpenelactones, or a flavan-3-ol;
   the at least one ECS related pathway anti-inflammatory compound that detectably or significantly modulates (preferably decreases) gene expression of IL-1beta, IL-1alpha(IL1a), IL-6, IL-8, NFKappaBeta (NFKB), TNFalpha (TNFa), and detectably or significantly modulates (preferably increases) gene expression of IL-10, or any combination thereof, is a hydroxyflavone, diterpene, triterpene or N-acetyl L-cysteine; and
   the at least one ECS related TRP pathway compound that detectably or significantly modulates (preferably increases) gene expression of TRPA1, TRPM8, TRPV4, TRPV6, and detectably or significantly modulates (preferably decreases) gene expression of TRPV1, TRPV3, or any combination thereof, is a poly-unsaturated fatty acid (PUFA) or a N-alkylamide (NAA).
3. The composition of embodiment 1 or 2, wherein
   the at least one direct endocannabinoid mimetic compound is curcumin, demethoxycurcumin, bisdemethoxycurcumin, tetrahydrocurcumin, eugenol, and its isomers and derivatives including isoeugenol, dihydroeugenol, ethyl guaiacol, or any combination thereof;
   the at least one indirect endocannabinoid mimetic compound that detectably or significantly modulates (preferably decreases) gene expression of FAAH is N-palmitoylethanolamide (PEA), N-oleoylethanolamide (OEA), Stearoylethanolamide (SEA), N-arachidonylethanolamide (AEA), Linoleoylethanolamide, Oleamide, Arachidonamide, or any combination thereof;
   the at least one indirect endocannabinoid mimetic compound that detectably or significantly modulates (preferably decreases) gene expression of MAGL is ginsenoside RC, N-palmitoylethanolamide (PEA), N-oleoylethanolamide (OEA), stearoylethanolamide (SEA), N-arachidonylethanolamide (AEA), linoleoylethanolamide, oleamide, arachidonamide, or any combination thereof;
   the at least one ECS related pathway anti-inflammatory compound that detectably or significantly modulates (preferably increases) gene expression of PPARgamma (PPARg), PPARalpha (PPARa), PPARbeta (PPARb), or any combination thereof is diosphenol, isomenthone, menthone, limonene, menthol, myrcene, linalool, pinene, camphor, honokiol, magnolol, resveratrol, diethylstilbestrol, or any combination thereof;
   the at least one ECS related pathway anti-inflammatory compound that detectably or significantly modulates (preferably decreases) gene expression of COX1 (i.e., PTGS1), COX2, iNOS, 5-LOX, 12-LOX, MMP1, or any combination thereof, is β caryophyllene, humulene, farnesene, farnesol, zingiberene, longifolene, copaene, patchoulol, ginkolide A, B, C, J or M, bilobalide, parthenolide, helenalin, lactucin, lactucopicrin, epigallocatechin gallate, catechin, epicatechin, gallocatechin, epigallocatechin, catechin gallate, epicatechin gallate, or any combination thereof;
   the at least one ECS related pathway anti-inflammatory compound that detectably or significantly modulates (preferably decreases) gene expression of IL-1beta, IL-1alpha(IL1a), IL-6, IL-8, NFKappaBeta (NFKB), TNFalpha (TNFa), and detectably or significantly modulates (preferably increases) gene expression of IL-10, or any combination thereof, is 7-hydroxyflavone, 3,7-dihydroxyflavone, quercetin, fisetin, apigenin, kaempferol, triptolide, rosmanol, carnosic acid, salvinorin A, forskolin, triterpene alcohols & triterpendiol monoesters (faradiol), N-acetyl L-cysteine, or any combination thereof; and the at least one ECS related TRP pathway compound that detectably or significantly modulates (preferably increases) gene expression of TRPA1, TRPM8, TRPV4, TRPV6, and detectably or significantly modulates (preferably decreases) gene expression of TRPV1, TRPV3 or any combination thereof, is docosahexaenoic acid (DHA), eicosapentaenoic acid (EPA), alpha-linolenic acid (ALA), eicosatetraenoic Acid (ETA), oleic acid, palmitoleic acid, vaccenic acid, dodeca-2E,4E,8Z,10Z-tetraenoic acid isobutylamide, dodeca-2E,4E-dienoic acid isobutylamide, or any combination thereof.
4. The composition of any one of embodiments 1-3, wherein at least one compound of the composition is contained in a natural extract, and wherein:
   when one or more of the direct endocannabinoid mimetic compounds is contained in a natural extract, the natural extract in each instance is a Curcuma longa, Curcuma xanthorrhiza, Curcuma zedoaria, Eugenia caryophyllata, Syzygium aromaticum, Myristica fragrans, Cinnamomum verum, Ocimum basilicum, or Laurus nobilis natural extract;
   when one or more of the indirect endocannabinoid mimetic compounds that detectably or significantly modulates (preferably decreases) gene expression of FAAH is contained in a natural extract, the natural extract in each instance is a Glycine max, Arachis hypogaea, Gallus gallus domesticus (egg oil) or Theobroma cacao natural extract;
   when one or more of the indirect endocannabinoid mimetic compounds that detectably or significantly modulates (preferably decreases) gene expression of MAGL is contained in a natural extract, the natural extract in each instance is a Panax notogensing (root) or Panex gensing (Ginseng), Glycine max, Arachis hypogaea, Gallus gallus domesticus (egg oil) or Theobroma cacao natural extract;
   when one or more of the ECS related pathway anti-inflammatory compounds that modulate (preferably increases) gene expression of PPARgamma (PPARg), PPARalpha (PPARa), PPARbeta (PPARb), or any combination thereof, is contained in a natural extract, the natural extract in each instance is a Agosthoma betulina, Agosthoma crenulata, Myristica fragrans, Melaleuca leucadendra L., Mentha longifolia, Syzygium polyanthum, Laurus nobilis, Humulus lupulus, Boswellia serrata, Zingiber officinale, Pinus longifolia, Pinus roxburghii, Ocimum basilicum L., Citrus bergamia, Artemisia californica, Cinnamomum camphora, Magnolia officinalis, Magnolia grandiflora, Magnolia dealbata, Magnolia biondii, Magnolia obovate, Vitis Vinifera L., or Vaccinium sp. natural extract;
   when one or more of the ECS related pathway anti-inflammatory compounds that detectably or significantly modulates (preferably decreases) gene expression of COX1 (i.e., PTGS1), COX2, iNOS, 5-LOX, 12-LOX, MMP1, or any combination thereof, is contained in a natural extract, the natural extract in each instance is a Bidens pilosa, Syzygium aromaticum (Eugenia caryophyllata), Piper nigrum, Perilla frutescens, Rosmarinus officinalis, Lindera benzoin, Centella asiatica, Angelica archangelica, Coleus barbatus, Origanum vulgare, Ptychopetalum olacoides, Ocimum basilicum, Salvia officinalis, Vitex agnus-castus, Petroselinum crispum, Coriandrum sativum, Boswellia sacra, Apium graveolens, Eucalyptus citriodora, Piper cubeba, Cinnamomum verum, Thymus vulgaris, Myrrhis odorata, Pinus sylvestris, Valeriana officinalis, Aesculus hippocastanum, Murraya koenigii, Tagetes minuta, Tamarindus indica, Melaleuca alternifolia, Mentha longifolia, Citrus limon, Ocimum tenuiflorum, Tagetes filifolia, Hedychium flavum, Eucalyptus tetraptera, Micromeria fruticosa, Salvia triloba, Artemisia annua, Salvia canariensis, Pogostemon cablin, Copaifera officinalis, Humulus lupulus, Curma longa, Curcuma xanthorrhiza, Curcuma zedoaria, Zingiber officinale, Copaifera langsdorfii, Citrus aurantiifolia, Citrus reticulata, Ginkgo biloba, Tanacetum parthenium, Arnica montana, Lactuca virosa lactucin, Camellia sinensis, Helianthemum glomeratum, Vaccinium oxycoccos, Fragaria ananassa, Rubus fruticosus, Actinidia deliciosa, Prunus avium, Pyrus sp., Prunus persica, Malus domestica, Persea americana, Carya illinoinensis, Pistacia vera, or Corylus avellana natural extract;
   when one or more of the ECS related pathway anti-inflammatory compounds that detectably or significantly modulates (preferably decreases) gene expression of IL-1beta, IL-1alpha(IL1a), IL-6, IL-8, NFKappaBeta (NFKB), TNFalpha (TNFa), and detectably or significantly modulates (preferably increases) gene expression of IL-10, or any combination thereof, is contained in a natural extract, the natural extract in each instance is a Daemonorops draco, Dracaena cochinchinensis, Camellia sinensis, Fragaria sp., Matricaria chamomilla, Petroselinum crispum, Allium cepa, Citrus Sinensis, Triticum aestivum, Aloe vera, Malus domestica, Tripterygium wilfordii, Salvia mellifera, Rosmarinus officinalis, Salvia officinalis, Salvia mellifera, or Salvia divinorum natural extract; and
   when one or more of the ECS related TRP pathway compounds that detectably or significantly modulates (preferably increases) gene expression of TRPA1, TRPM8, TRPV4, TRPV6, and detectably or significantly modulates (preferably decreases) gene expression of TRPV1, TRPV3, or any combination thereof, is contained in a natural extract, the natural extract in each instance is a Schizochytrium aggregatum (Algal Oil), Clupea pallasii (Pacific Herring oil), Oncorhynchus tshawytscha (Chinook Salmon oil), Euphausia sp. (Krill oil), Linum usitatissimum, Camelina sativa, Perilla frutescens, Juglans nigra, Olea europaea, Macadamia integrifolia, or Echinacea purpurea natural extract.
5. The composition of any one of embodiments 1-4, wherein:
   the at least one direct endocannabinoid mimetic compound is one or more curcuminoids;
   the at least one indirect endocannabinoid mimetic compound that detectably or significantly modulates (preferably decreases) gene expression of FAAH is one or more fatty acid amides;
   the at least one indirect endocannabinoid mimetic compound that detectably or significantly modulates (preferably decreases) gene expression of MAGL is one or more fatty acid amides;
   the at least one ECS related pathway anti-inflammatory compound that detectably or significantly modulates (preferably increases) gene expression of PPARgamma (PPARg), PPARalpha (PPARa), PPARbeta (PPARb), or any combination thereof is one or more monoterpenes or biphenols;
   the at least one ECS related pathway anti-inflammatory compound that detectably or significantly modulates (preferably decreases) gene expression of COX1 (i.e., PTGS1), COX2, iNOS, 5-LOX, 12-LOX, MMP1, or any combination thereof, is one or more sesquiterpenes, one or more flavan-3-ols, or any combination thereof;
   the at least one ECS related pathway anti-inflammatory compound that detectably or significantly modulates (preferably decreases) gene expression of IL-1beta, IL-1alpha(IL1a), IL-6, IL-8, NFKappaBeta (NFKB), TNFalpha (TNFa), and detectably or significantly modulates (preferably increases) gene expression of IL-10, or any combination thereof, is one or more hydroxyflavones, one or more diterpenes, or any combination thereof; and
   the at least one ECS related TRP pathway compound that detectably or significantly modulates (preferably increases) gene expression of TRPA1, TRPM8, TRPV4, TRPV6, and detectably or significantly modulates (preferably decreases) gene expression of TRPV1, TRPV3 or any combination thereof, is one or more PUFAs.
6. The composition of any one of embodiments 1-5, wherein
   the at least one direct endocannabinoid mimetic compound is curcumin, demethoxycurcumin, bisdemethoxycurcumin, tetrahydrocurcumin, or any combination thereof;
   the at least one indirect endocannabinoid mimetic compound that detectably or significantly modulates (preferably decreases) gene expression of FAAH is N-oleoylethanolamide (OEA), N-palmitoylethanolamide (PEA), or any combination thereof;
   the at least one indirect endocannabinoid mimetic compound that detectably or significantly modulates (preferably decreases) gene expression of MAGL is N-oleoylethanolamide (OEA), N-palmitoylethanolamide (PEA), or any combination thereof;
   the at least one ECS related pathway anti-inflammatory compound that detectably or significantly modulates (preferably increases) gene expression of PPARgamma (PPARg), PPARalpha (PPARa), PPARbeta (PPARb), or any combination thereof, is diosphenol, isomenthone, menthone, limonene, honokiol, magnolol, or any combination thereof;
   the at least one ECS related pathway anti-inflammatory compound that detectably or significantly modulates (preferably decreases) gene expression of COX1 (i.e., PTGS1), COX2, iNOS, 5-LOX, 12-LOX, MMP1, or any combination thereof, is β caryophyllene, epicatechin gallate, or a combination thereof;
   the at least one ECS related pathway anti-inflammatory compound that detectably or significantly modulates (preferably decreases) gene expression of IL-1beta, IL-1alpha(IL1a), IL-6, IL-8, NFKappaBeta (NFKB), TNFalpha (TNFa), and detectably or significantly modulates (preferably increases) gene expression of IL-10, or any combination thereof, is apigenin, triptolide, or a combination thereof; and
   the at least one ECS related TRP pathway compound that detectably or significantly modulates (preferably increases) gene expression of TRPA1, TRPM8, TRPV4, TRPV6, and detectably or significantly modulates (preferably decreases) gene expression of TRPV1, TRPV3 or any combination thereof, is docosahexaenoic acid (DHA), eicosapentaenoic acid (EPA), or a combination thereof
7. The composition of any one of embodiments 1-6, wherein at least one compound of the composition is contained in a natural extract, and wherein
   when one or more of the direct endocannabinoid mimetic compounds is contained in a natural extract, the natural extract in each instance is a Curcuma longa, Curcuma xanthorrhiza, or Curcuma zedoaria natural extracts;
   when one or more of the indirect endocannabinoid mimetic compounds that detectably or significantly modulates (preferably decreases) gene expression of FAAH is contained in a natural extract, the natural extract in each instance is a Theobroma cacao, Achyranthes aspera, Glycine max, Arachis hypogaea, or Gallus gallus domesticus (egg oil) natural extract;
   when one or more of the indirect endocannabinoid mimetic compounds that detectably or significantly modulates (preferably decreases) gene expression of MAGL is contained in a natural extract, the natural extract in each instance is Theobroma cacao, Achyranthes aspera, Glycine max, Arachis hypogaea, or Gallus gallus domesticus (egg oil) natural extract;
   when one or more of the ECS related pathway anti-inflammatory compounds that detectably or significantly modulates (preferably increase) gene expression of PPARgamma (PPARg), PPARalpha (PPARa), PPARbeta (PPARb), or any combination thereof, is contained in a natural extract, the natural extract in each instance is a Agathosma betulina, Agathosma crenulata, Magnolia officinalis, Magnolia grandiflora, Magnolia dealbata, Magnolia biondii, or Magnolia obovate natural extract;
   when one or more of the ECS related pathway anti-inflammatory compounds that detectably or significantly modulates (preferably decreases) gene expression of COX1 (i.e., PTGS1), COX2, iNOS, 5-LOX, 12-LOX, MMP1, or any combination thereof, is contained in a natural extract, the natural extract in each instance is a Bidens pilosa, Syzygium aromaticum (Eugenia caryophyllata), Piper nigrum, Perilla frutescens, Rosmarinus officinalis, Lindera benzoin, Centella asiatica, Angelica archangelica, Coleus barbatus, Origanum vulgare, Ptychopetalum olacoides, Ocimum basilicum, Salvia officinalis, Vitex agnus-castus, Petroselinum crispum, Coriandrum sativum, Boswellia sacra, Apium graveolens, Eucalyptus citriodora, Piper cubeba, Cinnamomum verum, Thymus vulgaris, Myrrhis odorata, Pinus sylvestris, Valeriana officinalis, Aesculus hippocastanum, Murraya koenigii, Tagetes minuta, Tamarindus indica, Melaleuca alternifolia, Mentha longifolia, Citrus limon, Ocimum tenuiflorum, Tagetes filifolia, Hedychium flavum, Eucalyptus tetraptera, Micromeria fruticosa, Salvia triloba, Artemisia annua, Salvia canariensis, Pogostemon cablin, Copaifera officinalis, Camellia sinensis, Helianthemum glomeratum, Vaccinium oxycoccos, Fragaria ananassa, Rubus fruticosus, Actinidia deliciosa, Prunus avium, Pyrus sp., Prunus persica, Malus domestica, Persea americana, Carya illinoinensis, Pistacia vera, or Corylus avellana natural extract;
   when one or more of the ECS related pathway anti-inflammatory compounds that detectably or significantly modulates (preferably decreases) gene expression of IL-1beta, IL-1alpha(IL1a), IL-6, IL-8, NFKappaBeta (NFKB), TNFalpha (TNFa), and detectably or significantly modulates (preferably increases) gene expression of IL-10, or any combination thereof, is contained in a natural extract, the natural extract in each instance is a Matricaria chamomilla, Petroselinum crispum, Allium cepa, Citrus Sinensis, Triticum aestivum, or Tripterygium wilfordii natural extract; and
   when one or more of the ECS related TRP pathway compounds that detectably or significantly modulates (preferably increases) gene expression of TRPA1, TRPM8, TRPV4, TRPV6, and detectably or significantly modulates (preferably decreases) gene expression of TRPV1, TRPV3 or any combination thereof, is contained in a natural extract, the natural extract in each instance is a Schizochytrium aggregatum, Clupea pallasii, Oncorhynchus tshawytscha, or Euphausia sp. natural extract.
8. The composition of any one of embodiments 1-7, wherein:
   the at least one direct endocannabinoid mimetic compound is one or more curcuminoids;
   the at least one indirect endocannabinoid mimetic compound that detectably or significantly modulates (preferably decreases) gene expression of FAAH is one or more fatty acid amides;
   the at least one indirect endocannabinoid mimetic compound that detectably or significantly modulates (preferably decreases) gene expression of MAGL is one or more fatty acid amides;
   the at least one ECS related pathway anti-inflammatory compound that detectably or significantly modulates (preferably increases) gene expression of PPARgamma (PPARg) is one or more monoterpenes, one or more biphenols, or any combination thereof;
   the at least one ECS related pathway anti-inflammatory compound that detectably or significantly modulates (preferably decreases) gene expression of COX1 (i.e., PTGS1), and/or MMP1 is one or more sesquiterpenes, one or more flavan-3-ols, or any combination thereof;
   the at least one ECS related pathway anti-inflammatory compound that detectably or significantly modulates (preferably decreases) gene expression of IL-1alpha(IL1a) and/or NFKB is one or more hydroxyflavones, one or more diterpenes, or any combination thereof; and
   the at least one ECS related TRP pathway compound that detectably or significantly modulates gene expression (preferably increases) of TRPA1, TRPM8, and that detectably or significantly modulates (preferably decreases) gene expression of TRPV1, or any combination thereof, is one or more PUFAs.
9. The composition of any one of embodiments 1-8, wherein
   the at least one direct endocannabinoid mimetic compound is curcumin
   the at least one indirect endocannabinoid mimetic compound that detectably or significantly modulates (preferably decreases) gene expression of FAAH is OEA, PEA, or a combination thereof;
   the at least one indirect endocannabinoid mimetic compound that detectably or significantly modulates (preferably decreases) gene expression of MAGL is OEA, PEA, or a combination thereof;
   the at least one ECS related pathway anti-inflammatory compound that detectably or significantly modulates (preferably increases) gene expression of PPARgamma (PPARg) is diosphenol, isomenthone, menthone, limonene, honokiol, magnolol, or any combination thereof;
   the at least one ECS related pathway anti-inflammatory compound that detectably or significantly modulates (preferably decreases) gene expression of COX1 (i.e., PTGS1), and/or MMP1 is β caryophyllene, epigallocatechin gallate, or a combination thereof;
   the at least one ECS related pathway anti-inflammatory compound that detectably or significantly modulates (preferably decreases) gene expression of IL-1alpha(IL1a) and/or NFKB is apigenin, triptolide, or a combination thereof; and
   the at least one ECS related TRP pathway compound that detectably or significantly modulates (preferably increases) gene expression of TRPA1, TRPM8, and that detectably or significantly modulates (preferably decreases) gene expression of TRPV1, or any combination thereof, is DHA, EPA, or any combination thereof.
10. The composition of any one of embodiments 1-9, wherein at least one compound of the composition is contained in a natural extract, and wherein
   when one or more of the direct endocannabinoid mimetic compounds is contained in a natural extract, the natural extract in each instance is a Curcuma longa, Curcuma xanthorrhiza, or Curcuma zedoaria natural extract;
   when one or more of the indirect endocannabinoid mimetic compounds that detectably or significantly modulates (preferably decreases) gene expression of FAAH is contained in a natural extract, the natural extract in each instance is a Theobroma cacao, Achyranthes aspera, Glycine max, Arachis hypogaea, or Gallus gallus domesticus (egg oil) natural extract;
   when one or more of the indirect endocannabinoid mimetic compounds that detectably or significantly modulates (preferably decreases) gene expression of MAGL is contained in a natural extract, the natural extract in each instance is Theobroma cacao, Achyranthes aspera, Glycine max, Arachis hypogaea, or Gallus gallus domesticus (egg oil) natural extract;
   when one or more of the ECS related pathway anti-inflammatory compounds that detectably or significantly modulates (preferably increases) gene expression of PPARgamma (PPARg) is contained in a natural extract, the natural extract in each instance is a Agathosma betulina, Agathosma crenulata, Magnolia officinalis, Magnolia grandiflora, Magnolia dealbata, Magnolia biondii, or Magnolia obovate natural extract;
   when one or more of the ECS related pathway anti-inflammatory compounds that detectably or significantly modulates (preferably decreases) gene expression of COX1 (i.e., PTGS1) and/or MMP1 is contained in a natural natural extract, the natural extract in each instance is a Bidens pilosa, Syzygium aromaticum (Eugenia caryophyllata), Piper nigrum, Perilla frutescens, Rosmarinus officinalis, Lindera benzoin, Centella asiatica, Angelica archangelica, Coleus barbatus, Origanum vulgare, Ptychopetalum olacoides, Ocimum basilicum, Salvia officinalis, Vitex agnus-castus, Petroselinum crispum, Coriandrum sativum, Boswellia sacra, Apium graveolens, Eucalyptus citriodora, Piper cubeba, Cinnamomum verum, Thymus vulgaris, Myrrhis odorata, Pinus sylvestris, Valeriana officinalis, Aesculus hippocastanum, Murraya koenigii, Tagetes minuta, Tamarindus indica, Melaleuca alternifolia, Mentha longifolia, Citrus limon, Ocimum tenuiflorum, Tagetes filifolia, Hedychium flavum, Eucalyptus tetraptera, Micromeria fruticosa, Salvia triloba, Artemisia annua, Salvia canariensis, Pogostemon cablin, Copaifera officinalis, Camellia sinensis, Helianthemum glomeratum, Vaccinium oxycoccos, Fragaria ananassa, Rubus fruticosus, Actinidia deliciosa, Prunus avium, Pyrus sp., Prunus persica, Malus domestica, Persea americana, Carya illinoinensis, Pistacia vera, or Corylus avellana natural extract;
   when one or more of the ECS related pathway anti-inflammatory compounds that detectably or significantly modulates (preferably decreases) gene expression of IL1a and/or NFKB is contained in a natural extract, the natural extract in each instance is a Matricaria chamomilla, Petroselinum crispum, Allium cepa, Citrus Sinensis, Triticum aestivum, or Tripterygium wilfordii natural extract; and
   when one or more of the ECS related TRP pathway compounds that detectably or significantly modulates (preferably increases) gene expression of TRPA1, TRPM8, and that detectably or significantly modulates (preferably decreases) gene expression of TRPV1, or any combination thereof, is contained in a natural extract, wherein the natural extract in each instance is a Schizochytrium aggregatum, Clupea pallasii, Oncorhynchus tshawytscha, or Euphausia sp. natural extract.
11. The composition of any one of embodiments 1-10, wherein
   the at least one direct endocannabinoid mimetic compound is curcumin;
   the at least one indirect endocannabinoid mimetic compound that detectably or significantly modulates (preferably decreases) gene expression of FAAH is PEA;
   the at least one indirect endocannabinoid mimetic compound that detectably or significantly modulates (preferably decreases) gene expression of MAGL is PEA;
   the at least one ECS related pathway anti-inflammatory compound that detectably or significantly modulates (preferably increases) gene expression of PPARgamma (PPARg) is diosphenol, limonene, isomenthone, menthone, or any combination thereof;
   the at least one ECS related pathway anti-inflammatory compound that detectably or significantly modulates (preferably decreases) gene expression of COX1 (i.e., PTGS1), and/or MMP1 is β caryophyllene;
   the at least one ECS related pathway anti-inflammatory compound that detectably or significantly modulates (preferably decreases) gene expression of IL-1alpha(IL1a) and/or NFKB is triptolide; and
   the at least one ECS related TRP pathway compound that detectably or significantly modulates (preferably increases) gene expression of TRPA1, TRPM8, and that detectably or significantly modulates (preferably decreases) gene expression of TRPV1, or any combination thereof, is DHA, EPA, or any combination thereof.
12. The composition of any one of embodiments 1-11, wherein at least one compound of the composition is contained in a natural extract, and wherein
   when one or more of the direct endocannabinoid mimetic compounds is contained in a natural extract, the natural extract in each instance is a Curcuma longa, Curcuma xanthorrhiza, or Curcuma zedoaria natural extract;
   when one or more of the indirect endocannabinoid mimetic compounds that detectably or significantly modulates (preferably decreases) gene expression of FAAH is contained in a natural extract, the natural extract in each instance is a Glycine max, Arachis hypogaea, or Gallus gallus domesticus (egg oil) natural extract;
   when one or more of the indirect endocannabinoid mimetic compounds that detectably or significantly modulates (preferably decreases) gene expression of MAGL is contained in a natural extract, the natural extract in each instance is Glycine max, Arachis hypogaea, or Gallus gallus domesticus (egg oil);
   when one or more of the ECS related pathway anti-inflammatory compounds that detectably or significantly modulates (preferably increases) gene expression of PPARgamma (PPARg) is contained in a natural extract, the natural extract in each instance is a Agathosma betulina or Agathosma crenulata natural extract;
   when one or more of the ECS related pathway anti-inflammatory compounds that detectably or significantly modulates (preferably decreases) gene expression of COX1 (i.e., PTGS1), and/or MMP1 is contained in a natural extract, the natural extract in each instance is a Bidens pilosa, Syzygium aromaticum (Eugenia caryophyllata), Piper nigrum, Perilla frutescens, Rosmarinus officinalis, Lindera benzoin, Centella asiatica, Angelica archangelica, Coleus barbatus, Origanum vulgare, Ptychopetalum olacoides, Ocimum basilicum, Salvia officinalis, Vitex agnus-castus, Petroselinum crispum, Coriandrum sativum, Boswellia sacra, Apium graveolens, Eucalyptus citriodora, Piper cubeba, Cinnamomum verum, Thymus vulgaris, Myrrhis odorata, Pinus sylvestris, Valeriana officinalis, Aesculus hippocastanum, Murraya koenigii, Tagetes minuta, Tamarindus indica, Melaleuca alternifolia, Mentha longifolia, Citrus limon, Ocimum tenuiflorum, Tagetes filifolia, Hedychium flavum, Eucalyptus tetraptera, Micromeria fruticosa, Salvia triloba, Artemisia annua, Salvia canariensis, Pogostemon cablin, or Copaifera officinalis natural extract;
   when one or more of the ECS related pathway anti-inflammatory compounds that detectably or significantly modulates (preferably decreases) gene expression of IL-1alpha and/or NFKB is contained in a natural extract, the natural extract in each instance is a Tripterygium wilfordii natural extract; and
   when one or more of the ECS related TRP pathway compounds that detectably or significantly modulates (preferably increases) gene expression of TRPA1, TRPM8, and that detectably or significantly modulates (preferably decreases) gene expression of TRPV1, or any combination thereof, is contained in a natural extract, the natural extract in each instance is a Schizochytrium aggregatum, Clupea pallasii, Oncorhynchus tshawytscha, or Euphausia sp. natural extract.
13. The composition of embodiments 1-12, wherein
   the at least one direct endocannabinoid mimetic compound is curcumin;
   the at least one indirect endocannabinoid mimetic compound that detectably or significantly modulates (preferably decreases) gene expression of FAAH is PEA;
   the at least one indirect endocannabinoid mimetic compound that detectably or significantly modulates (preferably decreases) gene expression of MAGL is OEA;
   the at least one ECS related pathway anti-inflammatory compound that detectably or significantly modulates (preferably decreases) gene expression of COX1 (i.e., PTGS1), and/or MMP1 is β caryophyllene;
   the at least one ECS related pathway anti-inflammatory compound that detectably or significantly modulates (preferably decreases) gene expression of IL-1alpha(IL1a) and/or NFKB is triptolide, 7-hydroxyflavone, or a combination thereof; and
   the at least one ECS related TRP pathway compound that detectably or significantly modulates (preferably increases) gene expression of TRPA1, TRPM8, and that detectably or significantly modulates (preferably decreases) gene expression of TRPV1, or any combination thereof, is DHA, EPA, or any combination thereof.
14. The composition of any one of embodiments 1-13, wherein at least one compound of the composition is contained in a natural extract, and wherein
   when one or more of the direct endocannabinoid mimetic compounds is contained in a natural extract, the natural extract in each instance is a Curcuma longa, Curcuma xanthorrhiza, or Curcuma zedoaria natural extract;
   when one or more of the indirect endocannabinoid mimetic compounds that detectably or significantly modulates (preferably decreases) gene expression of FAAH is contained in a natural extract, the natural extract in each instance is a Glycine max, Arachis hypogaea, or Gallus gallus domesticus (egg oil) natural extract;
   when one or more of the indirect endocannabinoid mimetic compounds that detectably or significantly modulates (preferably decreases) gene expression of MAGL is contained in a natural extract, the natural extract in each instance is Theobroma cacao or Achyranthes aspera;
   when one or more of the ECS related pathway anti-inflammatory compounds that detectably or significantly modulates (preferably decreases) gene expression of COX1 (i.e., PTGS1), and/or MMP1 is contained in a natural extract, the natural extract in each instance is a Bidens pilosa, Syzygium aromaticum (Eugenia caryophyllata), Piper nigrum, Perilla frutescens, Rosmarinus officinalis, Lindera benzoin, Centella asiatica, Angelica archangelica, Coleus barbatus, Origanum vulgare, Ptychopetalum olacoides, Ocimum basilicum, Salvia officinalis, Vitex agnus-castus, Petroselinum crispum, Coriandrum sativum, Boswellia sacra, Apium graveolens, Eucalyptus citriodora, Piper cubeba, Cinnamomum verum, Thymus vulgaris, Myrrhis odorata, Pinus sylvestris, Valeriana officinalis, Aesculus hippocastanum, Murraya koenigii, Tagetes minuta, Tamarindus indica, Melaleuca alternifolia, Mentha longifolia, Citrus limon, Ocimum tenuiflorum, Tagetes filifolia, Hedychium flavum, Eucalyptus tetraptera, Micromeria fruticosa, Salvia triloba, Artemisia annua, Salvia canariensis, Pogostemon cablin, or Copaifera officinalis natural extract;
   when one or more of the ECS related pathway anti-inflammatory compounds that detectably or significantly modulates (preferably decreases) gene expression of IL-1alpha and/or NFKB is contained in a natural extract, the natural extract in each instance is a Tripterygium wilfordii, Daemonorops draco, or Dracaena cochinchinensis natural extract; and
   when one or more of the ECS related TRP pathway compounds that detectably or significantly modulates (preferably increases) gene expression of TRPA1, TRPM8, and that detectably or significantly modulates (preferably decreases) gene expression of TRPV1, or any combination thereof, is contained in a natural extract, the natural extract in each instance is a Schizochytrium aggregatum (Algal Oil), Clupea pallasii (Pacific Herring oil), Oncorhynchus tshawytscha (Chinook Salmon oil), or Euphausia sp. (krill oil) natural extract.
15. The composition of embodiments 1-14, wherein
   the at least one direct endocannabinoid mimetic compound is curcumin;
   the at least one indirect endocannabinoid mimetic compound that detectably or significantly modulates (preferably decreases) gene expression of FAAH is PEA;
   the at least one indirect endocannabinoid mimetic compound that detectably or significantly modulates (preferably decreases) gene expression of MAGL is OEA;
   the at least one ECS related pathway anti-inflammatory compound that detectably or significantly modulates (preferably increases) gene expression of PPARgamma (PPARg) is honokiol, magnolol, or a combination thereof;
   the at least one ECS related pathway anti-inflammatory compound that detectably or significantly modulates (preferably decreases) gene expression of COX1 (i.e., PTGS1), and/or MMP1 is β caryophyllene, epigallocatechin gallate, or any combination thereof;
   the at least one ECS related pathway anti-inflammatory compound that detectably or significantly modulates (preferably decreases) gene expression of IL-1alpha(IL1a) and/or NFKB is triptolide, apigenin, or a combination thereof; and
   the at least one ECS related TRP pathway compound that detectably or significantly modulates (preferably increases) gene expression of TRPA1, TRPM8, and that detectably or significantly modulates (preferably decreases) gene expression of TRPV1, or any combination thereof, is DHA, EPA, or any combination thereof.
16. The composition of any one of embodiments 1-15, wherein at least one compound of the composition is contained in a natural extract, and wherein
   when one or more of the direct endocannabinoid mimetic compounds is contained in a natural extract, the natural extract in each instance is a Curcuma longa, Curcuma xanthorrhiza, or Curcuma zedoaria natural extract;
   when one or more of the indirect endocannabinoid mimetic compounds that detectably or significantly modulates (preferably decreases) gene expression of FAAH is contained in a natural extract, the natural extract in each instance is a Glycine max, Arachis hypogaea, or Gallus gallus domesticus (egg oil) natural extract;
   when one or more of the indirect endocannabinoid mimetic compounds that detectably or significantly modulates (preferably decreases) gene expression of MAGL is contained in a natural extract, the natural extract in each instance is Theobroma cacao or Achyranthes aspera;
   when one or more of the ECS related pathway anti-inflammatory compounds that detectably or significantly modulates (preferably increases) gene expression of PPARgamma (PPARg) is contained in a natural extract, the natural extract in each instance is a Magnolia officinalis, Magnolia grandiflora, Magnolia dealbata, Magnolia biondii, or Magnolia obovate natural extract;
   when one or more of the ECS related pathway anti-inflammatory compounds that detectably or significantly modulates (preferably decreases) gene expression of COX1 (i.e., PTGS1), and/or MMP1 is contained in a natural extract, the natural extract in each instance is a Bidens pilosa, Syzygium aromaticum (Eugenia caryophyllata), Piper nigrum, Perilla frutescens, Rosmarinus officinalis, Lindera benzoin, Centella asiatica, Angelica archangelica, Coleus barbatus, Origanum vulgare, Ptychopetalum olacoides, Ocimum basilicum, Salvia officinalis, Vitex agnus-castus, Petroselinum crispum, Coriandrum sativum, Boswellia sacra, Apium graveolens, Eucalyptus citriodora, Piper cubeba, Cinnamomum verum, Thymus vulgaris, Myrrhis odorata, Pinus sylvestris, Valeriana officinalis, Aesculus hippocastanum, Murraya koenigii, Tagetes minuta, Tamarindus indica, Melaleuca alternifolia, Mentha longifolia, Citrus limon, Ocimum tenuiflorum, Tagetes filifolia, Hedychium flavum, Eucalyptus tetraptera, Micromeria fruticosa, Salvia triloba, Artemisia annua, Salvia canariensis, Pogostemon cablin, Copaifera officinalis, Camellia sinensis, Helianthemum glomeratum, Vaccinium oxycoccos, Fragaria ananassa, Rubus fruticosus, Actinidia deliciosa, Prunus avium, Pyrus sp., Prunus persica, Malus domestica, Persea americana, Carya illinoinensis, Pistacia vera, or Corylus avellana natural extract;
   when one or more of the ECS related pathway anti-inflammatory compounds that detectably or significantly modulates (preferably decreases) gene expression of IL-1alpha and/or NFKB is contained in a natural extract, the natural extract in each instance is a Tripterygium wilfordii, Matricaria chamomilla, Petroselinum crispum, Allium cepa, Citrus Sinensis, or Triticum aestivum natural extract; and
   when one or more of the ECS related TRP pathway compounds that detectably or significantly modulates (preferably increases) gene expression of TRPA1, TRPM8, and that detectably or significantly modulates (preferably decreases) gene expression of TRPV1, or any combination thereof, is contained in a natural extract, the natural extract in each instance is a Schizochytrium aggregatum, Clupea pallasii, Oncorhynchus tshawytscha, or Euphausia sp. natural extract.
17. The composition of any one of embodiments 1-16, wherein:
   the at least one direct endocannabinoid mimetic compound is triptolide;
   the at least one indirect endocannabinoid mimetic compound that detectably or significantly modulates (preferably decreases) gene expression of FAAH is triptolide;
   the at least one indirect endocannabinoid mimetic compound that detectably or significantly modulates (preferably decreases) gene expression of MAGL is triptolide;
   the at least one ECS related pathway anti-inflammatory compound that detectably or significantly modulates (preferably increases) gene expression of PPARgamma (PPARg) is diosphenol;
   the at least one ECS related pathway anti-inflammatory compound that detectably or significantly modulates (preferably decreases) gene expression of COX1 (i.e., PTGS1), and/or MMP1 dodeca-2E,4E,8Z,10Z-tetraenoic acid isobutylamide, dodeca-2E,4E-dienoic acid isobutylamide, or a combination thereof;
   the at least one ECS related pathway anti-inflammatory compound that detectably or significantly modulates (preferably decreases) gene expression of IL-1alpha(IL1a) and/or NFKB is 7-hydroxyflavone; and
   the at least one ECS related TRP pathway compound that detectably or significantly modulates (preferably increases) gene expression of TRPA1, TRPM8, and that detectably or significantly modulates (preferably decreases) gene expression of TRPV1, or any combination thereof, is OEA.
18. The composition of any one of embodiments 1-17, wherein at least one compound of the composition is contained in a natural extract, and wherein
   when one or more of the direct endocannabinoid mimetic compounds is contained in a natural extract, the natural extract in each instance is a Tripterygium wilfordii natural extract;
   when one or more of the indirect endocannabinoid mimetic compounds that detectably or significantly modulates (preferably decreases) gene expression of FAAH is contained in a natural extract, the natural extract in each instance is a Tripterygium wilfordii natural extract;
   when one or more of the indirect endocannabinoid mimetic compounds that detectably or significantly modulates (preferably decreases) gene expression of MAGL is contained in a natural extract, the natural extract in each instance is a Tripterygium wilfordii natural extract;
   when one or more of the ECS related pathway anti-inflammatory compounds that detectably or significantly modulates (preferably increases) gene expression of PPARgamma (PPARg) is contained in a natural extract, the natural extract in each instance is a Agathosma betulina or Agathosma crenulata natural extract;
   when one or more of the ECS related pathway anti-inflammatory compounds that detectably or significantly modulates (preferably decreases) gene expression of COX1 (i.e., PTGS1), and/or MMP1 is contained in a natural extract, the natural extract in each instance is an Echinacea purpurea natural extract;
   when one or more of the ECS related pathway anti-inflammatory compounds that detectably or significantly modulates (preferably decreases) gene expression of IL-1alpha and/or NFKB is contained in a natural extract, the natural extract in each instance is a Daemonorops draco or Dracaena cochinchinensis natural extract; and
   when one or more of the ECS related TRP pathway compounds that detectably or significantly modulates (preferably increases) gene expression of TRPA1, TRPM8, and that detectably or significantly modulates (preferably decreases) gene expression of TRPV1, or any combination thereof, is contained in a natural extract, wherein the natural extract in each instance is a Theobroma cacao or Achyranthes aspera natural extract.
19. The composition of any one of embodiments 1-18, wherein:
   the at least one direct endocannabinoid mimetic compound is demethoxycurcumin, bisdemethoxycurcumin, tetrahydrocurcumin, eugenol, or any combination thereof;
   the at least one indirect endocannabinoid mimetic compound that detectably or significantly modulates (preferably decreases) gene expression of FAAH is N-oleoylethanolamide (OEA), oleamide, arachidonamide, or any combination thereof;
   the at least one indirect endocannabinoid mimetic compound that detectably or significantly modulates (preferably decreases) gene expression of MAGL is N-oleoylethanolamide (OEA), oleamide, arachidonamide, or any combination thereof;
   the at least one ECS related pathway anti-inflammatory compound that detectably or significantly modulates (preferably increases) gene expression of PPARgamma (PPARg) is diosphenol, limonene, isomenthone, menthone, resveratrol, or any combination thereof;
   the at least one ECS related pathway anti-inflammatory compound that detectably or significantly modulates (preferably decreases) gene expression of COX1 (i.e., PTGS1), and/or MMP1 is humulene (alpha-caryophyllene), ginkolide, bilobalide, helenalin, parthenolide, or any combination thereof;
   the at least one ECS related pathway anti-inflammatory compound that detectably or significantly modulates (preferably decreases) gene expression of IL-1alpha(IL1a) and/or NFKB is triptolide, carnosic acid, or any combination thereof; and
   the at least one ECS related TRP pathway compound that detectably or significantly modulates (preferably increases) gene expression of TRPA1, TRPM8, and that detectably or significantly modulates (preferably decreases) gene expression of TRPV1, or any combination thereof, is oleic acid, palmitoleic acid, vaccenic acid, or any combination thereof
20. The composition of any one of embodiments 1-19, wherein at least one compound of the composition is contained in a natural extract, and wherein
   when one or more of the direct endocannabinoid mimetic compounds is contained in a natural extract, the natural extract in each instance is a Curcuma longa, Curcuma xanthorrhiza, Curcuma zedoaria, Eugenia caryophyllata, Syzygium aromaticum, Myristica fragrans, Cinnamomum verum, Ocimum basilicum, or Laurus nobilis natural extract;
   when one or more of the indirect endocannabinoid mimetic compounds that detectably or significantly modulates (preferably decreases) gene expression of FAAH is contained in a natural extract, the natural extract in each instance is a Theobroma cacao or Achyranthes aspera natural extract;
   when one or more of the indirect endocannabinoid mimetic compounds that detectably or significantly modulates (preferably decreases) gene expression of MAGL is contained in a natural extract, the natural extract in each instance is a Theobroma cacao or Achyranthes aspera natural extract;
   when one or more of the ECS related pathway anti-inflammatory compounds that detectably or significantly modulates (preferably increases) gene expression of PPARgamma (PPARg) is contained in a natural extract, the natural extract in each instance is a Agathosma betulina, Agathosma crenulata, Vitis Vinifera L., or Vaccinium sp. natural extract;
   when one or more of the ECS related pathway anti-inflammatory compounds that detectably or significantly modulates (preferably decreases) gene expression of COX1 (i.e., PTGS1), and/or MMP1 is contained in a natural extract, the natural extract in each instance is a Humulus lupulus, Ginkgo biloba, Arnica montana or Tanacetum parthenium natural extract;
   when one or more of the ECS related pathway anti-inflammatory compounds that detectably or significantly modulates (preferably decreases) gene expression of IL-1alpha and/or NFKB is contained in a natural extract, the natural extract in each instance is a Tripterygium wilfordii, Salvia mellifera, Rosmarinus officinalis, or Salvia officinalis natural extract; and
   when one or more of the ECS related TRP pathway compounds that detectably or significantly modulates (preferably increases) gene expression of TRPA1, TRPM8, and that detectably or significantly modulates (preferably decreases) gene expression of TRPV1, or any combination thereof, is contained in a natural extract, wherein the natural extract in each instance is a Olea europaea, or Macadamia integrifolia natural extract.
21. The composition of any one of embodiments 1-20, wherein:
   the at least one direct endocannabinoid mimetic compound is eugenol;
   the at least one indirect endocannabinoid mimetic compound that detectably or significantly modulates (preferably decreases) gene expression of FAAH is oleamide, arachidonamide, or any combination thereof;
   the at least one indirect endocannabinoid mimetic compound that detectably or significantly modulates (preferably decreases) gene expression of MAGL is oleamide, arachidonamide, or any combination thereof;
   the at least one ECS related pathway anti-inflammatory compound that detectably or significantly modulates (preferably increases) gene expression of PPARgamma (PPARg) is diosphenol, resveratrol, or any combination thereof;
   the at least one ECS related pathway anti-inflammatory compound that detectably or significantly modulates (preferably decreases) gene expression of COX1 (i.e., PTGS1), and/or MMP1 is ginkolide B, bilobalide, helenalin, parthenolide, or any combination thereof;
   the at least one ECS related pathway anti-inflammatory compound that detectably or significantly modulates (preferably decreases) gene expression of IL-1alpha(IL1a) and/or NFKB is triptolide, carnosic acid, or any combination thereof; and
   the at least one ECS related TRP pathway compound that detectably or significantly modulates (preferably increases) gene expression of TRPA1, TRPM8, and that detectably or significantly modulates (preferably decreases) gene expression of TRPV1, or any combination thereof, is oleic acid, palmitoleic acid, vaccenic acid, or any combination thereof
22. The composition of any one of embodiments 1-21, wherein at least one compound of the composition is contained in a natural extract, and wherein
   when one or more of the direct endocannabinoid mimetic compounds is contained in a natural extract, the natural extract in each instance is a Eugenia caryophyllata, Syzygium aromaticum, Myristica fragrans, Cinnamomum verum, Ocimum basilicum, or Laurus nobilis natural extract;
   when one or more of the ECS related pathway anti-inflammatory compounds that detectably or significantly modulates (preferably increases) gene expression of PPARgamma (PPARg) is contained in a natural extract, the natural extract in each instance is a Agathosma betulina, Agathosma crenulat, Vitis Vinifera L. or Vaccinium sp. natural extract;
   one or more of the ECS related pathway anti-inflammatory compounds that detectably or significantly modulates (preferably decreases) gene expression of COX1 (i.e., PTGS1), and/or MMP1 is contained in a natural extract, wherein the natural extract in each instance is a Ginkgo biloba, Arnica montana, or Tanacetum parthenium natural extract;
   when one or more of the ECS related pathway anti-inflammatory compounds that detectably or significantly modulates (preferably decreases) gene expression of IL-1alpha and/or NFKB is contained in a natural extract, the natural extract in each instance is a Tripterygium wilfordii, Salvia mellifera, Rosmarinus officinalis, or Salvia officinalis natural extract; and
   when one or more of the ECS related TRP pathway compounds that detectably or significantly modulates (preferably increases) gene expression of TRPA1, TRPM8, and that detectably or significantly modulates (preferably decreases) gene expression of TRPV1, or any combination thereof, is contained in a natural extract, the natural extract in each instance is a Olea europaea, or Macadamia integrifolia natural extract.
23. The composition of any one of embodiments 1-22, wherein:
   the at least one direct endocannabinoid mimetic compound is tetrahhydrocurcumin, eugenol, or any combination thereof;
   the at least one indirect endocannabinoid mimetic compound that detectably or significantly modulates (preferably decreases) gene expression of FAAH is oleoylethanolamide;
   the at least one indirect endocannabinoid mimetic compound that detectably or significantly modulates (preferably decreases) gene expression of MAGL is oleoylethanolamide;
   the at least one ECS related pathway anti-inflammatory compound that detectably or significantly modulates (preferably increases) gene expression of PPARgamma (PPARg) is diosphenol;
   the at least one ECS related pathway anti-inflammatory compound that detectably or significantly modulates (preferably decreases) gene expression of COX1 (i.e., PTGS1), and/or MMP1 is ginkolide B;
   the at least one ECS related pathway anti-inflammatory compound that detectably or significantly modulates (preferably decreases) gene expression of IL-1alpha(IL1a) and/or NFKB is triptolide; and
   the at least one ECS related TRP pathway compound that detectably or significantly modulates (preferably increases) gene expression of TRPA1, TRPM8, and that detectably or significantly modulates (preferably decreases) gene expression of TRPV1, or any combination thereof, is oleic acid, palmitoleic acid, vaccenic acid, or any combination thereof
24. The composition of any one of embodiments 1-23, wherein at least one compound of the composition is contained in a natural extract, and wherein
   when one or more of the direct endocannabinoid mimetic compounds is contained in a natural extract, the natural extract in each instance is a Eugenia caryophyllata, Syzygium aromaticum, Myristica fragrans, Cinnamomum verum, Ocimum basilicum, or Laurus nobilis natural extract;
   when one or more of the indirect endocannabinoid mimetic compounds that detectably or significantly modulates (preferably decreases) gene expression of FAAH is contained in a natural extract, the natural extract in each instance is a Theobroma cacao or Achyranthes aspera natural extract;
   when one or more of the indirect endocannabinoid mimetic compounds that detectably or significantly modulates (preferably decreases) gene expression of MAGL is contained in a natural extract, the natural extract in each instance is a Theobroma cacao or Achyranthes aspera natural extract;
   when one or more of the ECS related pathway anti-inflammatory compounds that detectably or significantly modulates (preferably increases) gene expression of PPARgamma (PPARg) is contained in a natural extract, the natural extract in each instance is a Agathosma betulina or Agathosma crenulata natural extract;
   when one or more of the ECS related pathway anti-inflammatory compounds that detectably or significantly modulates (preferably decreases) gene expression of COX1 (i.e., PTGS1), and/or MMP1 is contained in a natural extract, the natural extract in each instance is a Ginkgo biloba natural extract;
   when one or more of the ECS related pathway anti-inflammatory compounds that detectably or significantly modulates (preferably decreases) gene expression of IL-1alpha and/or NFKB is contained in a natural extract, the natural extract in each instance is a Tripterygium wilfordii natural extract; and
   when one or more of the ECS related TRP pathway compounds that detectably or significantly modulates (preferably increases) gene expression of TRPA1, TRPM8, and that detectably or significantly modulates (preferably decreases) gene expression of TRPV1, or any combination thereof, is contained in a natural extract, the natural extract in each instance is a Olea europaea, or Macadamia integrifolia natural extract.
25. The composition of any one of embodiments 1-24, wherein:
   the at least one direct endocannabinoid mimetic compound is eugenol;
   the at least one indirect endocannabinoid mimetic compound that detectably or significantly modulates (preferably decreases) gene expression of FAAH is triptolide;
   the at least one indirect endocannabinoid mimetic compound that detectably or significantly modulates (preferably decreases) gene expression of MAGL is triptolide;
   the at least one ECS related pathway anti-inflammatory compound that detectably or significantly modulates (preferably increases) gene expression of PPARgamma (PPARg) is diosphenol;
   the at least one ECS related pathway anti-inflammatory compound that detectably or significantly modulates (preferably decreases) gene expression of COX1 (i.e., PTGS1), and/or MMP1 is dodeca-2E,4E,8Z,10Z-tetraenoic acid isobutylamide, dodeca-2E,4E-dienoic acid isobutylamide, or a combination thereof;
   the at least one ECS related pathway anti-inflammatory compound that detectably or significantly modulates (preferably decreases) gene expression of IL-1alpha(IL1a) and/or NFKB is 7-hydroxyflavone; and
   the at least one ECS related TRP pathway compound that detectably or significantly modulates (preferably increases) gene expression of TRPA1, TRPM8, and that detectably or significantly modulates (preferably decreases) gene expression of TRPV1, or any combination thereof, is OEA.
26. The composition of any one of embodiments 1-25, wherein at least one compound of the composition is contained in a natural extract, and wherein
   when one or more of the direct endocannabinoid mimetic compounds is contained in a natural extract, the natural extract in each instance is a Eugenia caryophyllata, Syzygium aromaticum, Myristica fragrans, Cinnamomum verum, Ocimum basilicum, or Laurus nobilis natural extract;
   when one or more of the indirect endocannabinoid mimetic compounds that detectably or significantly modulates (preferably decreases) gene expression of FAAH is contained in a natural extract, the natural extract in each instance is a Tripterygium wilfordii natural extract;
   when one or more of the indirect endocannabinoid mimetic compounds that detectably or significantly modulates (preferably decreases) gene expression of MAGL is contained in a natural extract, the natural extract in each instance is a Tripterygium wilfordii natural extract;
   when one or more of the ECS related pathway anti-inflammatory compounds that detectably or significantly modulates (preferably increases) gene expression of PPARgamma (PPARg) is contained in a natural extract, the natural extract in each instance is a Agathosma betulina or Agathosma crenulata natural extract;
   when one or more of the ECS related pathway anti-inflammatory compounds that detectably or significantly modulates (preferably decreases) gene expression of COX1 (i.e., PTGS1), and/or MMP1 is contained in a natural extract, the natural extract in each instance is a Echinacea purpurea natural extract;
   when one or more of the ECS related pathway anti-inflammatory compounds that detectably or significantly modulates (preferably decreases) gene expression of IL-1alpha and/or NFKB is contained in a natural extract, the natural extract in each instance is a Daemonorops draco or Dracaena cochinchinensis natural extract; and
   when one or more of the ECS related TRP pathway compounds that detectably or significantly modulates (preferably increases) gene expression of TRPA1, TRPM8, and that detectably or significantly modulates (preferably decreases) gene expression of TRPV1, or any combination thereof, is contained in a natural extract, the natural extract in each instance is a Theobroma cacao and Achyranthes aspera natural extract.
27. The composition of any one of embodiments 1-26, wherein the composition provides ECS related pathway beneficial gene expression for genes affecting skin matrix function measured by at least one gene selected from the group consisting of COL1A1, AP-1 (JUN), KLF4, ITGB1, and KGF/FGF7, where the compounds are preferably selected from the group consisting of curcumin, B-caryophyllene, N-palmitoylethanolamide, N-oleoylethanolamide, N-alkylamides, 7-hydroxyflavone, honokiol, magnolol, diosphenol, isomenthone, menthone, limonene, docosahexaenoic acid (DHA) eicosapentaenoic acid (EPA), triptolide, ginsenoside, epigallocatechin gallate, apigenin, pentacyclic triterpene alcohols and triterpendiol monoesters including faradiol esters, and eugenol.
28. The composition of any one of embodiments 1-27, wherein the composition provides ECS related pathway beneficial gene expression for genes affecting skin barrier function, lipid synthesis and antimicrobial properties measured by at least one gene selected from the group consisting of TLR2, CERS3, and FLG, where the compounds are preferably selected from the group consisting of curcumin, B-caryophyllene, N-palmitoylethanolamide, N-oleoylethanolamide, N-alkylamides, 7-hydroxyflavone, honokiol, magnolol, diosphenol, docosahexaenoic acid (DHA), eicosapentaenoic acid (EPA), triptolide, ginsenoside, epigallocatechin gallate, apigenin, pentacyclic triterpene alcohols and triterpendiol monoesters including faradiol esters, and eugenol.
29. The composition of any one of embodiments 1-28, wherein the composition provides ECS related pathway beneficial gene expression for genes affecting cell senescence measured by at least one gene selected from the group consisting of GLB1, CDKN2A, CDKN1A, TP53, MDM2, MAPK1, CASP8 where the compounds are preferably selected from the group consisting of curcumin, B-caryophyllene, N-palmitoylethanolamide, N-oleoylethanolamide, N-alkylamides, 7-hydroxyflavone, 3,7 dihydroxyflavone, honokiol, magnolol, diosphenol, isomenthone, menthone, limonene, triptolide, ginsenoside, epigallocatechin gallate, apigenin, pentacyclic triterpene alcohols and triterpendiol monoesters including faradiol esters, and eugenol.
30. The composition of any one of embodiments 1-29, wherein the composition causes an increase in gene expression of at least one gene selected from the group consisting of CB1 or CB2, wherein the increase in gene expression is greater than the total increase in gene expression individually on a cumulative basis for the compounds in the composition caused by the (a) at least one direct endocannabinoid mimetic compound, (b) and at least one indirect endocannabinoid mimetic compound, and (c) at least one ECS related pathway anti-inflammatory compound present in the composition (e.g., wherein the increase in gene expression is synergistic).
31. The composition of any one of embodiments 1-30, wherein the composition causes a decrease in gene expression of at least one gene selected from the group consisting of FAAH or MAGL, wherein the decrease in gene expression is greater than the total decrease in gene expression individually on a cumulative basis for the compounds in the composition caused by the (a) at least one direct endocannabinoid mimetic compound, and (b) at least one indirect endocannabinoid mimetic compound, and (c) at least one ECS related pathway anti-inflammatory compound present in the composition (e.g., wherein the increase in gene expression is synergistic).
32. The composition of any one of embodiments 1-31, wherein the composition causes a decrease in gene expression of at least one gene selected from the group consisting COX1 (i.e., PTGS1), IL-1alpha, NFKB and PPARg, wherein the decrease (or increase) in gene expression is greater than the total increase in gene expression individually on a cumulative basis for the compounds in the composition caused by the (a) at least one direct endocannabinoid mimetic compound, (b) and at least one indirect endocannabinoid mimetic compound, and (c) at least one ECS related pathway anti-inflammatory compound present in the composition (e.g., wherein the increase in gene expression is synergistic).
33. The composition of any one of embodiments 1-32, wherein the composition provides an ECS related TRP pathway nocioreceptor increase in gene expression for TRPA1, TRMP8, or a decrease in gene expression for TRPV1 greater than the respective nocioreceptor gene receptor increase or decrease response provided by camphor, menthol, or methyl salicylate and is a superior and/or synergistic gene expression increase compared to the individual compounds of the composition containing the direct endocannabinoid mimetic, the indirect endocannabinoid mimetic, the ECS related anti-inflammatory and the ECS related TRP pathway TRP nocioreceptor;
34. The composition of any one of embodiments 1-33, wherein the composition provides a pain relief effect greater than the pain relief effect provided by camphor, menthol, or methyl salicylate.
35. The composition of any one of embodiments 1-34, wherein the composition causes a beneficial change in gene expression of at least one gene selected from CB1, CB2, MAGL, FAAH, NFKB, IL1A, TRPM8, TRPV1, wherein the beneficial change in gene expression is greater than the beneficial change in gene expression caused by a composition comprising an equivalent concentration of cannabidiol.
36. The composition of any one of embodiments 1-35, wherein the composition is provided in a form selected from creams, lotions, solutions, sera, anhydrous preparations, emulsions, microemulsions, dermal patch, transdermal patch, multiple emulsions, gels, solid sticks, ointments, dry powders, sprays and aerosols.
37. The composition of any one of embodiments 1-36, wherein the amount of the at least one endocannabinoid mimetic-containing extract is about 0.01% to 30%, or 0.01% to 20%, or 0.01% to 10%, or 0.01% to 5.0% based on the weight of the composition.
38. A method of reducing or eliminating damage from intrinsic or extrinsic skin aging, comprising topically administering to a subject in need thereof a composition of any one of embodiments 1-37.
39. A method of improving the appearance of skin, comprising topically administering to a subject in need thereof a composition of any one of embodiments 1-37.
40. A method of reducing or eliminating a detrimental skin change, or promoting a positive skin change, comprising topically administering to a subject in need thereof a composition of any one of embodiments 1-37.
41. A method of reducing or eliminating a detrimental skin matrix change comprising topically administering to a subject in need thereof a composition a composition of any one of embodiments 1-37.
42. A method of detectably or significantly modulating cellular proliferation, differentiation, autophagy, apoptosis, and senescence for positive health beneficial effect, comprising topically administering to a subject in need thereof a composition of any one of embodiments 1-37.
43. A method of detectably or significantly modulating cellular senescence for health benefits, comprising topically administering to a subject in need thereof a composition of any one of embodiments 1-37.
44. A method of maintaining or improving barrier function of the skin, comprising topically administering to a subject in need thereof a composition of any one of embodiments 1-37.
45. A method of improving the skin microbiome comprising topically administering to a subject in need thereof a composition of any one of embodiments 1-37.
46. A method of improving wound healing, reducing inflammatory skin reaction, and improving skin recovery time post medical procedures that produce a wound healing and or inflammatory skin response including laser microdermabrasion, micro-needling, injectable fillers and toxins, fat reduction, chemical peels, comprising topically administering to a subject in need thereof a composition of any one of embodiments 1-37.
47. A method of reducing or eliminating pain, comprising topically administering to a subject in need thereof a composition of any one of embodiments 1-37.
48. A method of treating skin conditions including acne, hyperpigmentation, lines and wrinkles, psoriasis, dry skin, pruritis, or contact dermatitis, comprising topically administering to a subject in need thereof a composition of any one of embodiments 1-37.
49. A method of treating the detrimental effects of electromagnetic radiation and/or other inflammatory skin conditions, comprising topically administering to a subject in need thereof a composition of any one of embodiments 1-37.
50. A method of restoring ECS homeostasis equilibrium, comprising topically administering to a subject in need thereof a composition of any of embodiments 1-37.
51. The method of any of embodiments 38-50, wherein the subject is a mammal, preferably a human.
52. The method of any of embodiments 38-51, wherein the route of administration is topical.
53. The method of any of embodiments 38-52, wherein the route of administration is systemic.
54. The method of any of embodiments 38-53, wherein the composition is a cosmetic composition.
55. The method of any of embodiments 38-54, wherein the composition is a pharmaceutical composition.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. The term “or” herein is used in the inclusive sense of “and/or” unless clearly contradicted by statement, context, or plausibility. The occasional use of “and/or” herein has no effect on this construction of “or.” The terms defined herein, e.g., natural extract, are intended to have such meaning with or without capitalization in the specification.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A composition comprising:
a) at least one direct endocannabinoid mimetic compound, wherein each compound detectably or significantly modulates gene expression of the CB1 and/or CB2 gene;
b) at least one indirect endocannabinoid mimetic compound, wherein each compound:
1) detectably or significantly modulates gene expression of FAAH; and/or
2) detectably or significantly modulates gene expression of MAGL;
c) at least one ECS related pathway anti-inflammatory compound, wherein each compound
1) detectably or significantly modulates gene expression of PPARgamma (PPARg), PPARalpha (PPARa), PPARbeta (PPARb), or any combination thereof; and/or
2) detectably or significantly modulates gene expression of COX1 (i.e., PTGS1), COX2, iNOS, 5-LOX, 12-LOX, MMP1, or any combination thereof; and/or
3) detectably or significantly modulates gene expression of IL-1beta, IL-1alpha(IL1a), IL-6, IL-8, NFKappaBeta (NFKB), TNFalpha (TNFa), and modulates gene expression of IL-10, or any combination thereof; and
d) at least one ECS related TRP pathway compound, wherein each compound detectably or significantly modulates gene expression of TRPA1, TRPM8, TRPV4, TRPV6 and that modulates gene expression of TRPV1, TRPV3, or any combination thereof, and wherein gene expression in each case is measured in a cell exposed to the compound and is compared to the gene expression in a cell not exposed to the same compound.

2. The composition of claim 1, wherein
the at least one direct endocannabinoid mimetic compound is a curcuminoid or an allyl chain substituted guaiacol;
the at least one indirect endocannabinoid mimetic compound that detectably or significantly modulates gene expression of FAAH is a fatty acid amide;
the at least one indirect endocannabinoid mimetic compound that detectably or significantly modulates gene expression of MAGL is a ginsenoside or a fatty acid amide;
the at least one ECS related pathway anti-inflammatory compound that detectably or significantly modulates gene expression of PPARgamma (PPARg), PPARalpha (PPARa), PPARbeta (PPARb), is one or more monoterpenes, one or more biphenols, or any combination thereof;
the at least one ECS related pathway anti-inflammatory compound that detectably or significantly modulates gene expression of COX1 (i.e., PTGS1), COX2, iNOS, 5-LOX, 12-LOX, MMP1, or any combination thereof, is a sesquiterpene, a terpene lactones, or a flavan-3-ol;
the at least one ECS related pathway anti-inflammatory compound that detectably or significantly modulates gene expression of IL-1beta, IL-1alpha(IL1a), IL-6, IL-8, NFKappaBeta (NFKB), TNFalpha (TNFa), and detectably or significantly modulates gene expression of IL-10, or any combination thereof, is a hydroxyflavone, diterpene, triterpene or N-acetyl L-cysteine; and
the at least one ECS related TRP pathway compound that detectably or significantly modulates (preferably increases) gene expression of TRPA1, TRPM8, TRPV4, TRPV6, and detectably or significantly modulates gene expression of TRPV1, TRPV3, or any combination thereof, is a poly-unsaturated fatty acid (PUFA) or a N-alkylamide (NAA).

3. The composition of claim 2, wherein
the at least one direct endocannabinoid mimetic compound is curcumin, demethoxycurcumin, bisdemethoxycurcumin, tetrahydrocurcumin, eugenol, and its isomers and derivatives including isoeugenol, dihydroeugenol, ethyl guaiacol, or any combination thereof;
the at least one indirect endocannabinoid mimetic compound that detectably or significantly modulates gene expression of FAAH is N-palmitoylethanolamide (PEA), N-oleoylethanolamide (OEA), Stearoylethanolamide (SEA), N-arachidonylethanolamide (AEA), Linoleoylethanolamide, Oleamide, Arachidonamide, or any combination thereof;
the at least one indirect endocannabinoid mimetic compound that detectably or significantly modulates gene expression of MAGL is ginsenoside RC, N-palmitoylethanolamide (PEA), N-oleoylethanolamide (OEA), stearoylethanolamide (SEA), N-arachidonylethanolamide (AEA), linoleoylethanolamide, oleamide, arachidonamide, or any combination thereof;
the at least one ECS related pathway anti-inflammatory compound that detectably or significantly modulates gene expression of PPARgamma (PPARg), PPARalpha (PPARa), PPARbeta (PPARb), or any combination thereof is diosphenol, isomenthone, menthone, limonene, menthol, myrcene, linalool, pinene, camphor, honokiol, magnolol, resveratrol, diethylstilbestrol, or any combination thereof;
the at least one ECS related pathway anti-inflammatory compound that detectably or significantly modulates gene expression of COX1 (i.e, PTGS1), COX2, iNOS, 5-LOX, 12-LOX, MMP1, or any combination thereof, is β caryophyllene, humulene, farnesene, farnesol, zingiberene, longifolene, copaene, patchoulol, ginkolide A, B, C, J or M, bilobalide, parthenolide, helenalin, lactucin, lactucopicrin, epigallocatechin gallate, catechin, epicatechin, gallocatechin, epigallocatechin, catechin gallate, epicatechin gallate, or any combination thereof;
the at least one ECS related pathway anti-inflammatory compound that detectably or significantly modulates gene expression of IL-1beta, IL-1alpha(IL1a), IL-6, IL-8, NFKappaBeta (NFKB), TNFalpha (TNFa), and detectably or significantly modulates gene expression of IL-10, or any combination thereof, is 7-hydroxyflavone, 3,7-dihydroxyflavone, quercetin, fisetin, apigenin, kaempferol, triptolide, rosmanol, carnosic acid, salvinorin A, forskolin, triterpene alcohols & triterpendiol monoesters (faradiol), N-acetyl L-cysteine, or any combination thereof; and
the at least one ECS related TRP pathway compound that detectably or significantly modulates gene expression of TRPA1, TRPM8, TRPV4, TRPV6, and detectably or significantly modulates gene expression of TRPV1, TRPV3 or any combination thereof, is docosahexaenoic acid (DHA), eicosapentaenoic acid (EPA), alpha-linolenic acid (ALA), eicosatetraenoic Acid (ETA), oleic acid, palmitoleic acid, vaccenic acid, dodeca-2E,4E,8Z,10Z-tetraenoic acid isobutylamide, dodeca-2E,4E-dienoic acid isobutylamide, or any combination thereof.

4. The composition of claim 3, wherein at least one compound of the composition is contained in a natural extract, and wherein:
when one or more of the direct endocannabinoid mimetic compounds is contained in a natural extract, the natural extract in each instance is a Curcuma longa, Curcuma xanthorrhiza, Curcuma zedoaria, Eugenia caryophyllata, Syzygium aromaticum, Myristica fragrans, Cinnamomum verum, Ocimum basilicum, or Laurus nobilis natural extract;
when one or more of the indirect endocannabinoid mimetic compounds that detectably or significantly modulates gene expression of FAAH is contained in a natural extract, the natural extract in each instance is a Glycine max, Arachis hypogaea, Gallus gallus domesticus (egg oil) or Theobroma cacao natural extract;
when one or more of the indirect endocannabinoid mimetic compounds that detectably or significantly modulates gene expression of MAGL is contained in a natural extract, the natural extract in each instance is a Panax notogensing (root) or Panex gensing (Ginseng), Glycine max, Arachis hypogaea, Gallus gallus domesticus (egg oil) or Theobroma cacao natural extract;
when one or more of the ECS related pathway anti-inflammatory compounds that modulate gene expression of PPARgamma (PPARg), PPARalpha (PPARa), PPARbeta (PPARb), or any combination thereof, is contained in a natural extract, the natural extract in each instance is a Agosthoma betulina, Agosthoma crenulata, Myristica fragrans, Melaleuca leucadendra L., Mentha longifolia, Syzygium polyanthum, Laurus nobilis, Humulus lupulus, Boswellia serrata, Zingiber officinale, Pinus longifolia, Pinus roxburghii, Ocimum basilicum L., Citrus bergamia, Artemisia californica, Cinnamomum camphora, Magnolia officinalis, Magnolia grandiflora, Magnolia dealbata, Magnolia biondii, Magnolia obovate, Vitis Vinifera L., or Vaccinium sp. natural extract;
when one or more of the ECS related pathway anti-inflammatory compounds that detectably or significantly modulates gene expression of COX1 (i.e, PTGS1), COX2, iNOS, 5-LOX, 12-LOX, MMP1, or any combination thereof, is contained in a natural extract, the natural extract in each instance is a Bidens pilosa, Syzygium aromaticum (Eugenia caryophyllata), Piper nigrum, Perilla frutescens, Rosmarinus officinalis, Lindera benzoin, Centella asiatica, Angelica archangelica, Coleus barbatus, Origanum vulgare, Ptychopetalum olacoides, Ocimum basilicum, Salvia officinalis, Vitex agnus-castus, Petroselinum crispum, Coriandrum sativum, Boswellia sacra, Apium graveolens, Eucalyptus citriodora, Piper cubeba, Cinnamomum verum, Thymus vulgaris, Myrrhis odorata, Pinus sylvestris, Valeriana officinalis, Aesculus hippocastanum, Murraya koenigii, Tagetes minuta, Tamarindus indica, Melaleuca alternifolia, Mentha longifolia, Citrus limon, Ocimum tenuiflorum, Tagetes filifolia, Hedychium flavum, Eucalyptus tetraptera, Micromeria fruticosa, Salvia triloba, Artemisia annua, Salvia canariensis, Pogostemon cablin, Copaifera officinalis, Humulus lupulus, Curma longa, Curcuma xanthorrhiza, Curcuma zedoaria, Zingiber officinale, Copaifera langsdorfii, Citrus aurantiifolia, Citrus reticulata, Ginkgo biloba, Tanacetum parthenium, Arnica montana, Lactuca virosa lactucin, Camellia sinensis, Helianthemum glomeratum, Vaccinium oxycoccos, Fragaria ananassa, Rubus fruticosus, Actinidia deliciosa, Prunus avium, Pyrus sp., Prunus persica, Malus domestica, Persea americana, Carya illinoinensis, Pistacia vera, or Corylus avellana natural extract;
when one or more of the ECS related pathway anti-inflammatory compounds that detectably or significantly modulates gene expression of IL-1beta, IL-1alpha(IL1a), IL-6, IL-8, NFKappaBeta (NFKB), TNFalpha (TNFa), and detectably or significantly modulates gene expression of IL-10, or any combination thereof, is contained in a natural extract, the natural extract in each instance is a Daemonorops draco, Dracaena cochinchinensis, Camellia sinensis, Fragaria sp., Matricaria chamomilla, Petroselinum crispum, Allium cepa, Citrus Sinensis, Triticum aestivum, Aloe vera, Malus domestica, Tripterygium wilfordii, Salvia mellifera, Rosmarinus officinalis, Salvia officinalis, Salvia mellifera, or Salvia divinorum natural extract; and
when one or more of the ECS related TRP pathway compounds that detectably or significantly modulates gene expression of TRPA1, TRPM8, TRPV4, TRPV6, and detectably or significantly modulates gene expression of TRPV1, TRPV3, or any combination thereof, is contained in a natural extract, the natural extract each instance is a Schizochytrium aggregatum (Algal Oil), Clupea pallasii (Pacific Herring oil), Oncorhynchus tshawytscha (Chinook Salmon oil), Euphausia sp. (Krill oil), Linum usitatissimum, Camelina sativa, Perilla frutescens, Juglans nigra, Olea europaea, Macadamia integrifolia, or Echinacea purpurea natural extract.

5. The composition of claim 1, wherein:
the at least one direct endocannabinoid mimetic compound is one or more curcuminoids;
the at least one indirect endocannabinoid mimetic compound that detectably or significantly modulates gene expression of FAAH is one or more fatty acid amides;
the at least one indirect endocannabinoid mimetic compound that detectably or significantly modulates gene expression of MAGL is one or more fatty acid amides or ginsenosides;
the at least one ECS related pathway anti-inflammatory compound that detectably or significantly modulates gene expression of PPARgamma (PPARg) is one or more monoterpenes, one or more biphenols, or any combination thereof;
the at least one ECS related pathway anti-inflammatory compound that detectably or significantly modulates gene expression of COX1 (i.e, PTGS1), and/or MMP1 is one or more sesquiterpenes, one or more flavan-3-ols, one or more terpene lactones, or any combination thereof;
the at least one ECS related pathway anti-inflammatory compound that detectably or significantly modulates gene expression of IL-1alpha(IL1a) and/or NFKB is one or more hydroxyflavones, one or more diterpenes, or any combination thereof; and
the at least one ECS related TRP pathway compound that detectably or significantly modulates gene expression of TRPA1, TRPM8, and that detectably or significantly modulates gene expression of TRPV1, or any combination thereof, is one or more N-alkylamides or PUFAs.

6. The composition of claim 7, wherein
the at least one direct endocannabinoid mimetic compound is curcumin,
the at least one indirect endocannabinoid mimetic compound that detectably or significantly modulates gene expression of FAAH is OEA, PEA, or a combination thereof;
the at least one indirect endocannabinoid mimetic compound that detectably or significantly modulates gene expression of MAGL is ginsenoside RC, OEA, PEA, or a combination thereof;
the at least one ECS related pathway anti-inflammatory compound that detectably or significantly modulates gene expression of PPARgamma (PPARg) is diosphenol, isomenthone, menthone, limonene, honokiol, magnolol, or any combination thereof;
the at least one ECS related pathway anti-inflammatory compound that detectably or significantly modulates gene expression of COX1 (i.e, PTGS1), and/or MMP1 is β caryophyllene, epigallocatechin gallate, or a combination thereof;
the at least one ECS related pathway anti-inflammatory compound that detectably or significantly modulates gene expression of IL-1alpha(IL1a) and/or NFKB is apigenin, triptolide, or a combination thereof; and
the at least one ECS related TRP pathway compound that detectably or significantly modulates gene expression of TRPA1, TRPM8, and that detectably or significantly modulates gene expression of TRPV1, or any combination thereof, is DHA, EPA, dodeca-2E,4E,8Z,10Z-tetraenoic acid isobutylamide, dodeca-2E,4E-dienoic acid isobutylamide, or any combination thereof.

7. The composition of claim 6, wherein at least one compound of the composition is contained in a natural extract, and wherein
when one or more of the direct endocannabinoid mimetic compounds is contained in a natural extract, the natural extract in each instance is a Curcuma longa, Curcuma xanthorrhiza, or Curcuma zedoaria natural extract;
when one or more of the indirect endocannabinoid mimetic compounds that detectably or significantly modulates gene expression of FAAH is contained in a natural extract, the natural extract in each instance is a Theobroma cacao, Achyranthes aspera, Glycine max, Arachis hypogaea, or Gallus gallus domesticus (egg oil) natural extract;
when one or more of the indirect endocannabinoid mimetic compounds that detectably or significantly modulates gene expression of MAGL is contained in a natural extract, the natural extract in each instance is Panax notogensing (root), Panex gensing (Ginseng), Theobroma cacao, Achyranthes aspera, Glycine max, Arachis hypogaea, or Gallus gallus domesticus (egg oil) natural extract;
when one or more of the ECS related pathway anti-inflammatory compounds that detectably or significantly modulates gene expression of PPARgamma (PPARg) is contained in a natural extract, the natural extract in each instance is a Agathosma betulina, Agathosma crenulata, Magnolia officinalis, Magnolia grandiflora, Magnolia dealbata, Magnolia biondii, or Magnolia obovate natural extract;
when one or more of the ECS related pathway anti-inflammatory compounds that detectably or significantly modulates gene expression of COX1 (i.e, PTGS1) and/or MMP1 is contained in a natural extract, the natural extract in each instance is a Bidens pilosa, Syzygium aromaticum (Eugenia caryophyllata), Piper nigrum, Perilla frutescens, Rosmarinus officinalis, Lindera benzoin, Centella asiatica, Angelica archangelica, Coleus barbatus, Origanum vulgare, Ptychopetalum olacoides, Ocimum basilicum, Salvia officinalis, Vitex agnus-castus, Petroselinum crispum, Coriandrum sativum, Boswellia sacra, Apium graveolens, Eucalyptus citriodora, Piper cubeba, Cinnamomum verum, Thymus vulgaris, Myrrhis odorata, Pinus sylvestris, Valeriana officinalis, Aesculus hippocastanum, Murraya koenigii, Tagetes minuta, Tamarindus indica, Melaleuca alternifolia, Mentha longifolia, Citrus limon, Ocimum tenuiflorum, Tagetes filifolia, Hedychium flavum, Eucalyptus tetraptera, Micromeria fruticosa, Salvia triloba, Artemisia annua, Salvia canariensis, Pogostemon cablin, Copaifera officinalis, Camellia sinensis, Helianthemum glomeratum, Vaccinium oxycoccos, Fragaria ananassa, Rubus fruticosus, Actinidia deliciosa, Prunus avium, Pyrus sp., Prunus persica, Malus domestica, Persea americana, Carya illinoinensis, Pistacia vera, or Corylus avellana natural extract;
when one or more of the ECS related pathway anti-inflammatory compounds that detectably or significantly modulates gene expression of IL1a and/or NFKB is contained in a natural extract, the natural extract in each instance is a Matricaria chamomilla, Petroselinum crispum, Allium cepa, Citrus Sinensis, Triticum aestivum, or Tripterygium wilfordii natural extract; and
when one or more of the ECS related TRP pathway compounds that detectably or significantly modulates gene expression of TRPA1, TRPM8, and that detectably or significantly modulates gene expression of TRPV1, or any combination thereof, is contained in a natural extract, wherein the natural extract each instance is a Echinacea pupurea, Schizochytrium aggregatum, Clupea pallasii, Oncorhynchus tshawytscha, or Euphausia sp. natural extract.

8. The composition of claim 1, wherein
the at least one direct endocannabinoid mimetic compound is curcumin;
the at least one indirect endocannabinoid mimetic compound that detectably or significantly modulates (preferably decreases) gene expression of FAAH is N-oleoylethanolamide (OEA);
the at least one indirect endocannabinoid mimetic compound that detectably or significantly modulates (preferably decreases) gene expression of MAGL is ginsenoside RC;
the at least one ECS related pathway anti-inflammatory compound that detectably or significantly modulates (preferably increases) gene expression of PPARgamma (PPARg) is honokiol, magnolol, or any combination thereof;
the at least one ECS related pathway anti-inflammatory compound that detectably or significantly modulates (preferably decreases) gene expression of COX1 (i.e, PTGS1), MMP1, or any combination thereof, is β caryophyllene;
the at least one ECS related pathway anti-inflammatory compound that detectably or significantly modulates (preferably decreases) gene expression of IL-1alpha(IL1a), NFKappaBeta (NFKB), or any combination thereof, is apigenin; and
the at least one ECS related TRP pathway compound that detectably or significantly modulates (preferably increases) gene expression of TRPA1, TRPM8, or significantly modulates (preferably decreases) gene expression of TRPV1, TRPV3 or any combination thereof, is 10Z-tetraenoic acid isobutylamide, dodeca-2E,4E-dienoic acid isobutylamide, or any combination thereof.

9. The composition of claim 8, wherein at least one compound of the composition is contained in a natural extract, and wherein
when one or more of the direct endocannabinoid mimetic compounds is contained in a natural extract, the natural extract in each instance is a Curcuma longa, Curcuma xanthorrhiza, or Curcuma zedoaria natural extract;
when one or more of the indirect endocannabinoid mimetic compounds that detectably or significantly modulates (preferably decreases) gene expression of FAAH is contained in a natural extract, the natural extract in each instance is a Theobroma cacao natural extract;
when one or more of the indirect endocannabinoid mimetic compounds that detectably or significantly modulates (preferably decreases) gene expression of MAGL is contained in a natural extract, the natural extract in each instance is a Panax notogensing (root) or Panex gensing (Ginseng) natural extract;
when one or more of the ECS related pathway anti-inflammatory compounds that modulate (preferably increases) gene expression of PPARgamma (PPARg), is contained in a natural extract, the natural extract in each instance is a Magnolia officinalis natural extract;
when one or more of the ECS related pathway anti-inflammatory compounds that detectably or significantly modulates (preferably decreases) gene expression of COX1 (i.e, PTGS1), MMP1, or any combination thereof, is contained in a natural extract, the natural extract in each instance is a Bidens pilosa, Syzygium aromaticum (Eugenia caryophyllata), Piper nigrum, Perilla frutescens, Rosmarinus officinalis, Lindera benzoin, Centella asiatica, Angelica archangelica, Coleus barbatus, Origanum vulgare, Ptychopetalum olacoides, Ocimum basilicum, Salvia officinalis, Vitex agnus-castus, Petroselinum crispum, Coriandrum sativum, Boswellia sacra, Apium graveolens, Eucalyptus citriodora, Piper cubeba, Cinnamomum verum, Thymus vulgaris, Myrrhis odorata, Pinus sylvestris, Valeriana officinalis, Aesculus hippocastanum, Murraya koenigii, Tagetes minuta, Tamarindus indica, Melaleuca alternifolia, Mentha longifolia, Citrus limon, Ocimum tenuiflorum, Tagetes filifolia, Hedychium flavum, Eucalyptus tetraptera, Micromeria fruticosa, Salvia triloba, Artemisia annua, Salvia canariensis, Pogostemon cablin, Copaifera officinalis, Humulus lupulus, Curma longa, Curcuma xanthorrhiza, Curcuma zedoaria, Zingiber officinale, Copaifera langsdorfii, Citrus aurantiifolia, Citrus reticulata, Ginkgo biloba, Tanacetum parthenium, Arnica montana, Lactuca virosa lactucin, Camellia sinensis, Helianthemum glomeratum, Vaccinium oxycoccos, Fragaria ananassa, Rubus fruticosus, Actinidia deliciosa, Prunus avium, Pyrus sp., Prunus persica, Malus domestica, Persea americana, Carya illinoinensis, Pistacia vera, or Corylus avellana natural extract;
when one or more of the ECS related pathway anti-inflammatory compounds that detectably or significantly modulates (preferably decreases) gene expression of IL-1alpha(IL1a), NFKappaBeta (NFKB), or any combination thereof, is contained in a natural extract, the natural extract in each instance is a Matricaria chamomilla natural extract; and
when one or more of the ECS related TRP pathway compounds that detectably or significantly modulates (preferably increases) gene expression of TRPA1, TRPM8, detectably or significantly modulates (preferably decreases) gene expression of TRPV1, or any combination thereof, is contained in a natural extract, the natural extract in each instance is a Echinacea purpurea natural extract.

10. The composition of claim 1, wherein
the at least one direct endocannabinoid mimetic compound is curcumin;
the at least one indirect endocannabinoid mimetic compound that detectably or significantly modulates gene expression of FAAH is PEA;
the at least one indirect endocannabinoid mimetic compound that detectably or significantly modulates gene expression of MAGL is OEA;
the at least one ECS related pathway anti-inflammatory compound that detectably or significantly modulates gene expression of PPARgamma (PPARg) is diosphenol, honokiol, magnolol, or a combination thereof;
the at least one ECS related pathway anti-inflammatory compound that detectably or significantly modulates gene expression of COX1 (i.e, PTGS1), and/or MMP1 is β caryophyllene, epigallocatechin gallate, or any combination thereof;
the at least one ECS related pathway anti-inflammatory compound that detectably or significantly modulates gene expression of IL-1alpha(IL1a) and/or NFKB is triptolide, apigenin, or a combination thereof; and
the at least one ECS related TRP pathway compound that detectably or significantly modulates gene expression of TRPA1, TRPM8, and that detectably or significantly modulates gene expression of TRPV1, or any combination thereof, is DHA, EPA, or any combination thereof.

11. The composition of claim 10, wherein at least one compound of the composition is contained in a natural extract, and wherein
when one or more of the direct endocannabinoid mimetic compounds is contained in a natural extract, the natural extract in each instance is a Curcuma longa, Curcuma xanthorrhiza, or Curcuma zedoaria natural extract;
when one or more of the indirect endocannabinoid mimetic compounds that detectably or significantly modulates gene expression of FAAH is contained in a natural extract, the natural extract in each instance is a Glycine max, Arachis hypogaea, or Gallus gallus domesticus (egg oil) natural extract;
when one or more of the indirect endocannabinoid mimetic compounds that detectably or significantly modulates gene expression of MAGL is contained in a natural extract, the natural extract in each instance is Theobroma cacao or Achyranthes aspera;
when one or more of the ECS related pathway anti-inflammatory compounds that detectably or significantly modulates gene expression of PPARgamma (PPARg) is contained in a natural extract, the natural extract in each instance is a Magnolia officinalis, Magnolia grandiflora, Magnolia dealbata, Magnolia biondii, Magnolia obovate, Agathosma betulina or Agathosma crenulata natural extract;
when one or more of the ECS related pathway anti-inflammatory compounds that detectably or significantly modulates gene expression of COX1 (i.e., PTGS1), and/or MMP1 is contained in a natural extract, the natural extract in each instance is a Bidens pilosa, Syzygium aromaticum (Eugenia caryophyllata), Piper nigrum, Perilla frutescens, Rosmarinus officinalis, Lindera benzoin, Centella asiatica, Angelica archangelica, Coleus barbatus, Origanum vulgare, Ptychopetalum olacoides, Ocimum basilicum, Salvia officinalis, Vitex agnus-castus, Petroselinum crispum, Coriandrum sativum, Boswellia sacra, Apium graveolens, Eucalyptus citriodora, Piper cubeba, Cinnamomum verum, Thymus vulgaris, Myrrhis odorata, Pinus sylvestris, Valeriana officinalis, Aesculus hippocastanum, Murraya koenigii, Tagetes minuta, Tamarindus indica, Melaleuca alternifolia, Mentha longifolia, Citrus limon, Ocimum tenuiflorum, Tagetes filifolia, Hedychium flavum, Eucalyptus tetraptera, Micromeria fruticosa, Salvia triloba, Artemisia annua, Salvia canariensis, Pogostemon cablin, Copaifera officinalis, Camellia sinensis, Helianthemum glomeratum, Vaccinium oxycoccos, Fragaria ananassa, Rubus fruticosus, Actinidia deliciosa, Prunus avium, Pyrus sp., Prunus persica, Malus domestica, Persea americana, Carya illinoinensis, Pistacia vera, or Corylus avellana natural extract;
when one or more of the ECS related pathway anti-inflammatory compounds that detectably or significantly modulates gene expression of IL-1alpha and/or NFKB is contained in a natural extract, the natural extract in each instance is a Tripterygium wilfordii, Matricaria chamomilla, Petroselinum crispum, Allium cepa, Citrus Sinensis, or Triticum aestivum natural extract; and
when one or more of the ECS related TRP pathway compounds that detectably or significantly modulates gene expression of TRPA1, TRPM8, and that detectably or significantly modulates gene expression of TRPV1, or any combination thereof, is contained in a natural extract, the natural extract in each instance is a Schizochytrium aggregatum, Clupea pallasii, Oncorhynchus tshawytscha, or Euphausia sp. natural extract.

12. The composition of claim 1, wherein:
the at least one direct endocannabinoid mimetic compound is eugenol;
the at least one indirect endocannabinoid mimetic compound that detectably or significantly modulates gene expression of FAAH is oleamide, arachidonamide, or any combination thereof;
the at least one indirect endocannabinoid mimetic compound that detectably or significantly modulates gene expression of MAGL is oleamide, arachidonamide, or any combination thereof;
the at least one ECS related pathway anti-inflammatory compound that detectably or significantly modulates gene expression of PPARgamma (PPARg) is diosphenol, resveratrol, or any combination thereof;
the at least one ECS related pathway anti-inflammatory compound that detectably or significantly modulates gene expression of COX1 (i.e., PTGS1), and/or MMP1 is ginkolide B, bilobalide, helenalin, parthenolide, or any combination thereof;
the at least one ECS related pathway anti-inflammatory compound that detectably or significantly modulates gene expression of IL-1alpha(IL1a) and/or NFKB is triptolide, carnosic acid, or any combination thereof; and
the at least one ECS related TRP pathway compound that detectably or significantly modulates gene expression of TRPA1, TRPM8, and that detectably or significantly modulates gene expression of TRPV1, or any combination thereof, is oleic acid, palmitoleic acid, vaccenic acid or any combination thereof.

13. The composition of claim 12, wherein at least one compound of the composition is contained in a natural extract, and wherein
when one or more of the direct endocannabinoid mimetic compounds is contained in a natural extract, the natural extract in each instance is a Eugenia caryophyllata, Syzygium aromaticum, Myristica fragrans, Cinnamomum verum, Ocimum basilicum, or Laurus nobilis natural extract;
when one or more of the ECS related pathway anti-inflammatory compounds that detectably or significantly modulates gene expression of PPARgamma (PPARg) is contained in a natural extract, the natural extract in each instance is a Agathosma betulina, Agathosma crenulat, Vitis Vinifera L. or Vaccinium sp. natural extract;
one or more of the ECS related pathway anti-inflammatory compounds that detectably or significantly modulates gene expression of COX1 (i.e., PTGS1), and/or MMP1 is contained in a natural extract, wherein the natural extract in each instance is a Ginkgo biloba, Arnica montana, or Tanacetum parthenium natural extract;
when one or more of the ECS related pathway anti-inflammatory compounds that detectably or significantly modulates gene expression of IL-1alpha and/or NFKB is contained in a natural extract, the natural extract in each instance is a Tripterygium wilfordii, Salvia mellifera, Rosmarinus officinalis, or Salvia officinalis natural extract; and
when one or more of the ECS related TRP pathway compounds that detectably or significantly modulates gene expression of TRPA1, TRPM8, and that detectably or significantly modulates gene expression of TRPV1, or any combination thereof, is contained in a natural extract, the natural extract in each instance is a Olea europaea, or Macadamia integrifolia natural extract.

14. The composition of claim 1, wherein the composition provides ECS related pathway beneficial gene expression for genes affecting skin matrix function measured by at least one gene selected from the group consisting of COL1A1, AP-1 (JUN), KLF4, ITGB1, and KGF/FGF7, where the compounds are preferably selected from the group consisting of curcumin, B-caryophyllene, N-palmitoylethanolamide, N-oleoylethanolamide, N-alkylamides, 7-hydroxyflavone, honokiol, magnolol, diosphenol, isomenthone, menthone, limonene, docosahexaenoic acid (DHA) eicosapentaenoic acid (EPA), triptolide, ginsenoside, epigallocatechin gallate, apigenin, pentacyclic triterpene alcohols and triterpendiol monoesters including faradiol esters, and eugenol.

15. The composition of claim 1, wherein the composition provides ECS related pathway beneficial gene expression for genes affecting skin barrier function, lipid synthesis and antimicrobial properties measured by at least one gene selected from the group consisting of TLR2, CERS3 and FLG, the compounds are preferably selected from the group consisting of curcumin, B-caryophyllene, N-palmitoylethanolamide, N-oleoylethanolamide, N-alkylamides, 7-hydroxyflavone, honokiol, magnolol, diosphenol, docosahexaenoic acid (DHA), eicosapentaenoic acid (EPA), triptolide, ginsenoside, epigallocatechin gallate, apigenin, pentacyclic triterpene alcohols and triterpendiol monoesters including faradiol esters, and eugenol.

16. The composition of claim 1, wherein the composition is provided in a form selected from creams, lotions, solutions, sera, anhydrous preparations, emulsions, microemulsions, dermal patch, transdermal patch, multiple emulsions, gels, solid sticks, ointments, dry powders, sprays and aerosols.

17. The composition of claim 1, wherein the amount of the at least one endocannabinoid mimetic-containing extract is about 0.01% to 30%, or 0.01% to 20%, or 0.01% to 10%, or 0.01% to 5.0%, 0.01% to 1.0% based on the weight of the composition.

18. A method of reducing or eliminating a detrimental skin change, or promoting a positive skin change, comprising topically administering to a subject in need thereof a composition of claim 1.

19. A method of detectably or significantly modulating cellular proliferation, differentiation, autophagy, apoptosis, and senescence for positive health beneficial effect, comprising topically administering to a subject in need thereof a composition of claim 1.

20. A method of improving wound healing, reducing inflammatory skin reaction, and improving skin recovery time post medical procedures that produce a wound healing and or inflammatory skin response including laser microdermabrasion, micro-needling, injectable fillers and toxins, fat reduction, chemical peels, comprising topically administering to a subject in need thereof a composition of claim 1.

21. A method of reducing or eliminating pain, comprising topically administering to a subject in need thereof a composition of claim 1.

22. A method of treating skin conditions including acne, hyperpigmentation, lines and wrinkles, psoriasis, dry skin, pruritis, contact dermatitis, comprising topically administering to a subject in need thereof a composition of claim 1.

23. A method of restoring ECS homeostasis equilibrium and thereby improving general skin health benefits, comprising topically administering to a subject in need thereof a composition of claim 1.
